(19) United States
(12) Patent Application Publication
(10) Pub. No.: US 2025/0382322 A1
(43) Pub. Date: Dec. 18, 2025

(54) DIAGNOSTICS FOR DETECTING ECTO-5'-NUCLEOTIDASE (CD73)

(71) Applicant: EBERHARD KARLS UNIVERSITÄT TÜBINGEN, Tübingen (DE)

(72) Inventors: Anna JUNKER, Münster (DE); DOBELMANN Clemens, Münster (DE); HERMANN Sven, Münster (DE); Sonja SCHELHAAS, Münster (DE); Stefan WAGNER, Münster (DE); Georg ROLSHOVEN, Bonn (DE); Constanze Cerine SCHMIES, Bonn (DE); Laura SCHÄKEL, Bonn (DE); Riham IDRIS, Bonn (DE); Katharina SYLVESTER, Bonn (DE); Haneen AL-HROUB, Bonn (DE); Christa E. MÜLLER, Bonn (DE); Eugen POTAPTSCHUK, Bonn (DE)

(73) Assignee: EBERHARD KARLS UNIVERSITÄT TÜBINGEN, Tübingen (DE)

(21) Appl. No.: 18/838,807

(22) PCT Filed: Feb. 17, 2023
(86) PCT No.: PCT/EP2023/054046

§ 371 Date: Aug. 15, 2024

(30) Foreign Application Priority Data
Feb. 17, 2022 (EP) 22157311.6

(51) Int. Cl. C07H 19/167; A61K 51/04; A61P 35/00; C07H 23/00

(52) U.S. Cl. CPC C07H 19/167; A61K 51/0491; A61P 35/00; C07H 23/00; A61K 2121/00

(57) ABSTRACT

The present invention relates to radio- and fluorescence-labeled compounds, as well as their use as Diagnostics for Detecting Ecto-5′-Nucleotidase (CD73). The invention is further directed to a pharmaceutical composition comprising said compounds as well as to the compounds and the pharmaceutical composition for use in a method of diagnosis of a disease associated with increased or decreased CD73-expression as well as in the treatment of a disease associated with increased CD73-expression.

### CROSS-REFERENCE TO RELATED APPLICATIONS

The present invention is filed under 35 U.S.C. § 371 as the U.S. national phase of International Application No. PCT/EP2023/054046, filed on February 17, 223, which designated the U.S. and claims the right of priority of European Patent Application No. 22157311.6, filed on Feb. 17, 2022. The entire disclosures of the above-identified priority applications are hereby fully incorporated herein by reference.

### TECHNICAL FIELD OF THE INVENTION

The present invention relates to radio- and fluorescence-labeled compounds, as well as their use as Diagnostics for Detecting Ecto-5′-Nucleotidase (CD73). The invention is further directed to a pharmaceutical composition comprising said compounds as well as to the compounds and the pharmaceutical composition for use in a method of diagnosis of a disease associated with increased CD73-expression as well as in the treatment of a disease associated with increased CD73-expression.

### BACKGROUND ART

Ecto-5′-nucleotidase (CD73, eN) is an enzyme that catalyzes the dephosphorylation of extracellular AMP to adenosine.¹ It can be found on the surface of many different cell lines like endothelia, stromal and cells of the immune system like lymphocytes and regulatory T-cells. Furthermore, CD73 is highly overexpressed on various tumor cells such as bladder, colon, ovarian, melanoma, pancreatic and breast cancer.^{2,3} Stress factors like hypoxia (HIF-1α), proinflammatory factors (TGF-β, IFNs, TNF, IL-1β), low pH, low glucose levels and expression of Wnt/β-catenin, which can often be found in the tumor microenvironment, are promoting the expression of CD73.^{4, 5} Especially in triple negative breast cancer, lacking estrogen receptors (ER) that were demonstrated to downregulate the expression of CD73,⁶ the survival of patients is poor due to the upregulation of CD73.^{7, 8} The extracellular production of adenosine activates colocalized P1 receptors (P1R's) such as, e.g. A_{2A} and A_{2B} receptors,^{3, 9} thereby leading to an acceleration of tumor growth, metastasis and angiogenesis, and suppression of the infiltration of immune cells into solid tumors and thus the immune response through P1 receptor-mediated activity.^{10, 11} The inhibition of CD73 and simultaneous antagonism of A_{2A} or A_{2B} receptors, for example, leads to improved anti-cancer effects. The anti-tumor resistance is reduced and immune responses are improved, while metastasis, angiogenesis and tumor growth are reduced or delayed.^{11, 12} Therefore, CD73 represents a novel, promising target for checkpoint inhibition in cancer immunotherapy. In autoimmune diseases, e.g. multiple sclerosis, rheumatoid arthritis and systemic lupus erythematosus, CD73 expression is often altered compared to healthy controls. Therefore, CD73 expression may be a biomarker for these diseases and for monitoring therapies.

CD73 inhibition can be achieved using monoclonal antibodies or small molecule inhibitors. For example, MEDI9447, an antibody developed as a therapeutic agent against pancreatic cancer, is already being tested in clinical trials.¹³ Antibodies often have the disadvantage of being expensive and lacking the capability of infiltration into solid tumors. Moreover, they often do not lead to a complete inhibition of enzymatic activity. There are also different classes of small molecule inhibitors developed. While sulfonamides,^{14, 15} polyphenols,¹⁶ and anthraquinones^{17, 18} show only low inhibitory activities, nucleotide-derived CD73 inhibitors are highly potent.¹⁹⁻²²

The Müller group at the University of Bonn pioneered in the development of potent and selective CD73 inhibitors based on the ADP analog AMPCP (AOPCP).¹ This work led to the discovery of CD73 inhibitors with very high, even subnanomolar inhibitory potency, high chemical and metabolic stability, and high selectivity.¹⁻⁴ It could be shown that 2-chloro- and N⁶-benzyl-substitution of the adenine core structure leads to potent AMPCP-derived CD73 inhibitors.^{2, 3} Following the initial seminal work¹⁻⁴ the analysis of the structure-activity relationships (SARs) has been extended to develop further AMPCP derivatives and analogs. Bioisosteric replacement of the adenine ring in combination with suitable substituents was well tolerated, e.g., shifting or replacing ring nitrogen atoms or replacing the bicyclic purine by a monocyclic or tricyclic ring system e.g. a pyrimidine ring.⁵⁻¹⁰ This culminated in the development of AB680⁸ (see FIG. 1, compound III) by Arcus Biosciences, showing a similar substitution pattern as early compounds developed by Bhattarai & Müller.⁴ AB680 was selected by Arcus Biosciences for clinical trials, and preliminary results from a phase II study in prostate cancer look promising.¹¹

A published study on the first fluorescence-labeled CD73 inhibitors showed that the p-position of the N⁶-benzyl moiety represents a suitable position for further derivatization, even with large substituents. The introduction of a bulky fluorescein residue attached via a linker moiety was well tolerated leading to potent, selective, metabolically stable fluorescein-labeled CD73 inhibitors.¹²

There is a need for further fluorescence- and radio-labeled CD73 ligands.

### SUMMARY OF THE INVENTION

In a first aspect, the invention is directed to a compound according to general formula (I)

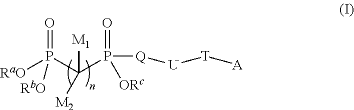

wherein
R^{a}, R^{b} and R^{c} are independently selected from the group consisting of H, —(C₁-C₆)alkyl, —(C₆-C₁₀)aryl, —C(O)(C₁-C₆)alkyl, —(C₁-C₆)alkyl(C₆-C₁₀)heteroaryl, —(C₆-C₁₀)heteroaryl and —C(O)aryl;
M₁ and M₂ are independently selected from the group consisting of H, —OH and halogen;
n is 1 to 6, preferably 1 to 3, more preferably 1;
Q is selected from the group consisting of O, S, CH₂, NH, preferably O;
U is selected from the group consisting of O, S, CH₂, (CH₂)₂, NH, preferably CH₂;
T is selected from the group consisting of

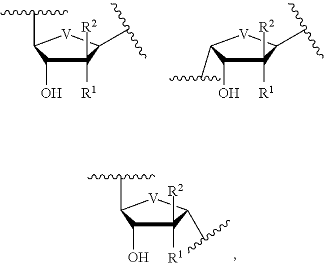

preferably

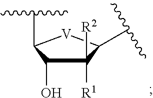

V is selected from the group consisting of O, NH, S, CH₂; preferably O
R¹ and R² are independently selected from the group consisting of H, OH, SH, —O(C₁-C₆)alkyl, —S(C₁-C₆)alkyl, —NH₂, —NH(C₁-C₆)alkyl, —N₃ and halogen, preferably H and —OH;
A is selected from the group consisting of

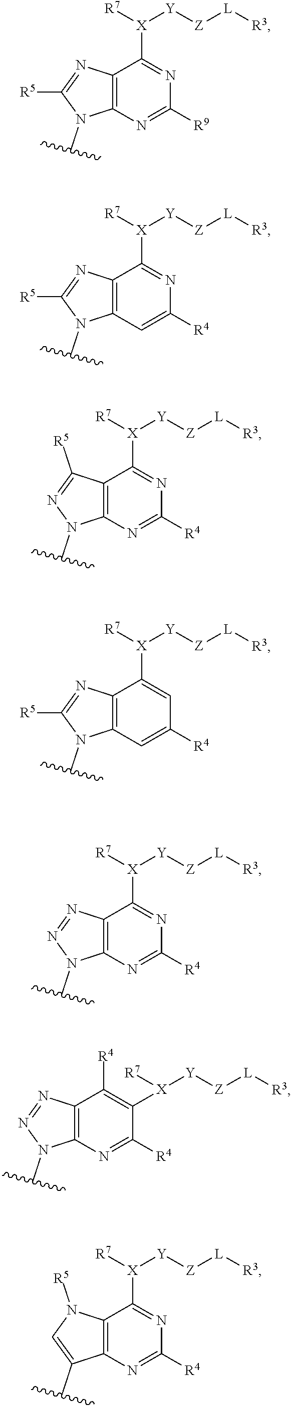

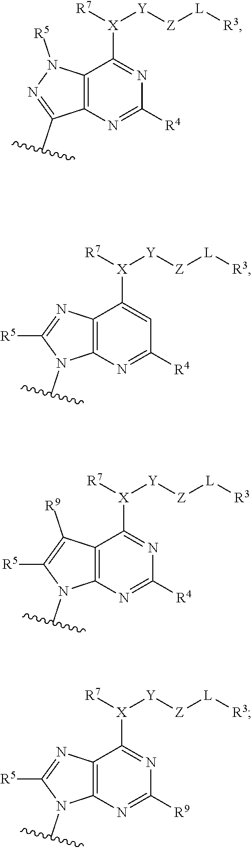

preferably

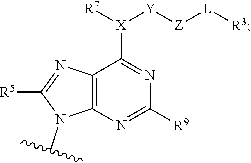

X is selected from the group consisting of O, S, N,

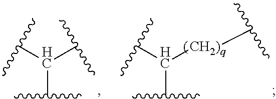

preferably N
q is 0 to 6, preferably 0 to 4, more preferably 0
Y is selected from the group consisting of —(C₆-C₁₀)aryl-, —(CH₂)ₚ—, —(CH₂)ₚC(O)—, —(C₆-C₁₀)heteroaryl(CH₂)ₚ—, —(C₆-C₁₀)heteroaryl-, —(CH₂)ₚ(C₆-C₁₀)aryl-, and —(C₆-C₁₀)arylC(O)—, preferably —(CH₂)ₚ—, or —(C₆-C₁₀)arylC(O)—
Z is selected from the group consisting of

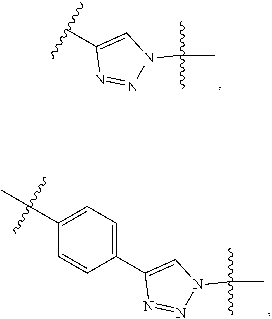 [SMILES: *c1cn(*)nn1 ; *c1ccc(-c2cn(*)nn2)cc1]

H, NH, O, S,

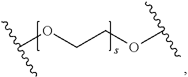

—(CH₂)ₚ—, —(C₆-C₁₀)aryl —(C₆-C₁₀)aryl(CH₂)ₚ, —C(O)(CH₂)ₚ—, —(C₆-C₁₀)heteroaryl(CH₂)ₚ—, —(C₆-C₁₀)heteroaryl-, —(C₆-C₁₀)arylC(O)—, preferably

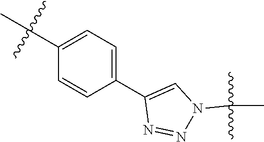 [SMILES: *c1ccc(-c2cn(*)nn2)cc1]

and —(C₆-C₁₀)arylC(O)—, wherein optionally the aryl groups may be substituted by one or more substituents selected from the group consisting of —(C₁-C₆)alkyl, —(C₂-C₆)alkynyl, -halogen, -trifluoromethyl, —OH, —SH, —NH₂, —SO₂NH₂, —(C₁-C₆)alkylOH, —O(C₁-C₆)alkyl, —SO₃H, —(CH₂)₁₋₆COOH, —COOH, —C(O)NH₂, —SO₃(C₁-C₆)alkyl, —(C₅-C₆)arylCH₂C(O)—, —C(O)NH(CH₂)ₒNH₂;
s is an integer from 1 to 60, preferably 1 to 50, more preferably 2 to 30;
o is an integer from 1 to 8, preferably 1 to 4, most preferably 4;
p is an integer from 1 to 6, preferably 1 to 4, most preferably 4;
L comprises L¹ and L², with L¹ connected to L², L¹-L²
L¹ is selected from the group consisting of absent, —(CH₂)_{q}—, —C(O)NH(CH₂)ₚNH—, —(CH₂)_{q}(C₅-C₁₀)aryl-, —(C₁-C₁₀)alkynyl-, —(C₆-C₁₀)aryl(CH₂)ₚ—, 1-halo-1-vinyl, —(C₆-C₁₀)heteroarylenyl(CH₂)ₚ—, —(C₆-C₁₀)heteroaryl-, and —(C₆-C₁₀)arylC(O)—,

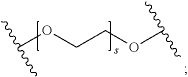

s is an integer from 1 to 60, preferably 1 to 50, more preferably 2 to 30;
q is an integer from 1 to 10, preferably 1 to 6, most preferably 5;
L² is selected from the group consisting of absent,

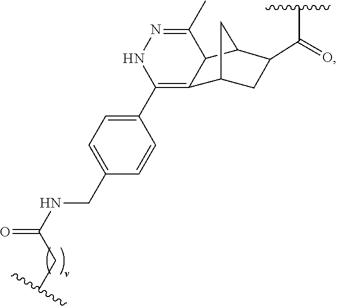

v, is 1 to 9, preferably 1 to 7 and

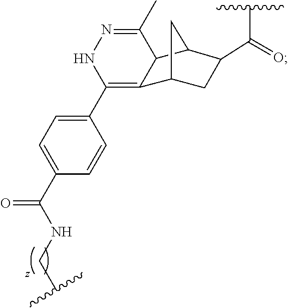

z is 1 to 6, preferably 1 to 5, more preferably 5; and/or
R³ is selected from the group consisting of
i),

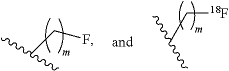

m is an integer of from 2 to 10; or
ii)

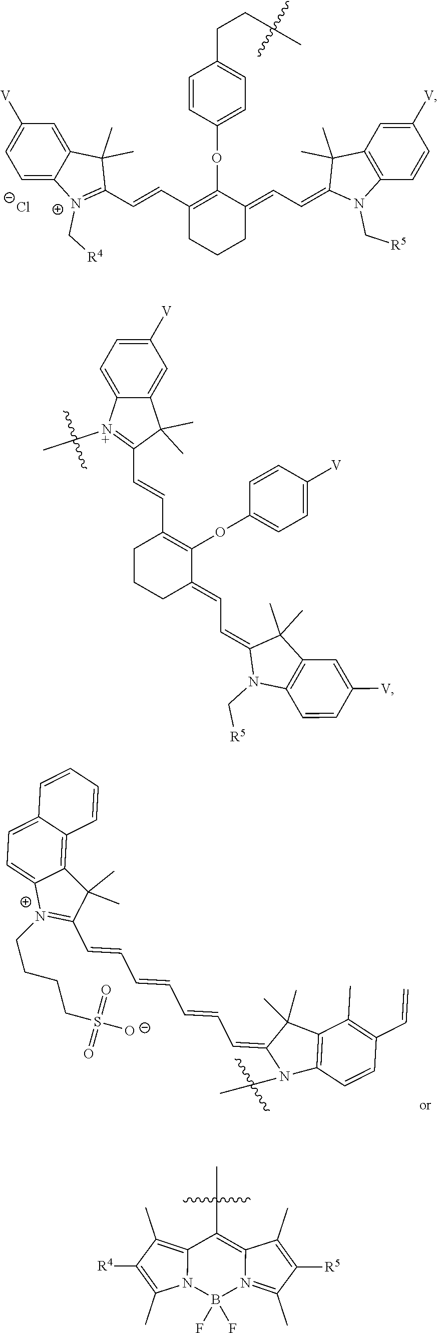

or
iii) a fluorophore moiety selected from the group consisting of FITC, Fluorescein, NBD, Dansyl, Squaraine Rotaxane, Bodipy FL, Bodipy TR, Bodipy 630/650 X, Bodipy 650/655 X, Texas Red, Cy5, 1-pyrene, EVOBlue 30, Alexa Fluor 532, Alexa Fluor 488-5, 488-6, or mixture thereof, Tamra, Tamra 5/6-X-SE, Alexa Fluor 488 azide 5 isomer, Alexa Fluor 488 5 isomer, Alexa Fluor 488 5/6 mixed isomers, NIR dye 700, NIR dye 800, Janelia Fluor 549 amide, Janelia Fluor 646 amide and derivates, analogs and related fluorophores thereof; or
iv) a chelating moiety binding a radioactive metal,
wherein the chelating moiety is selected from the group consisting of

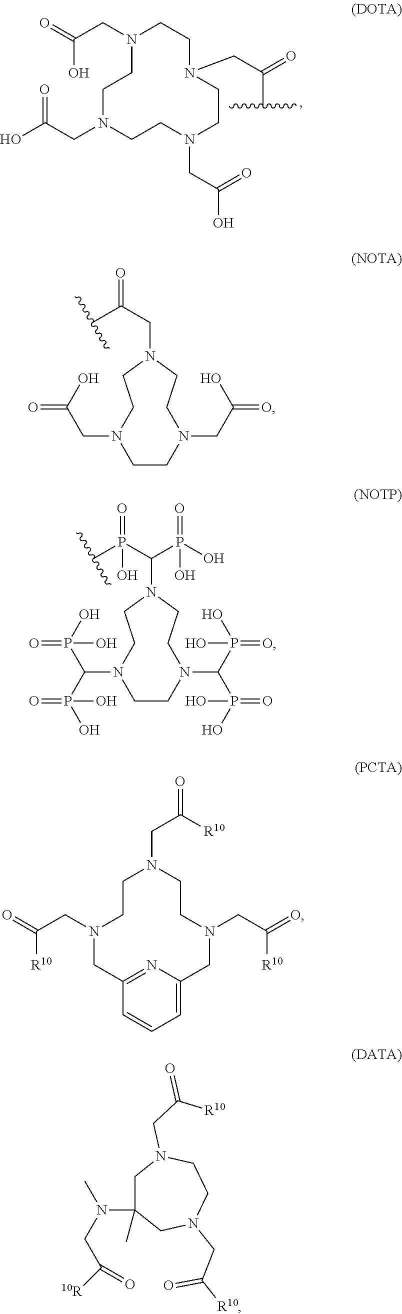

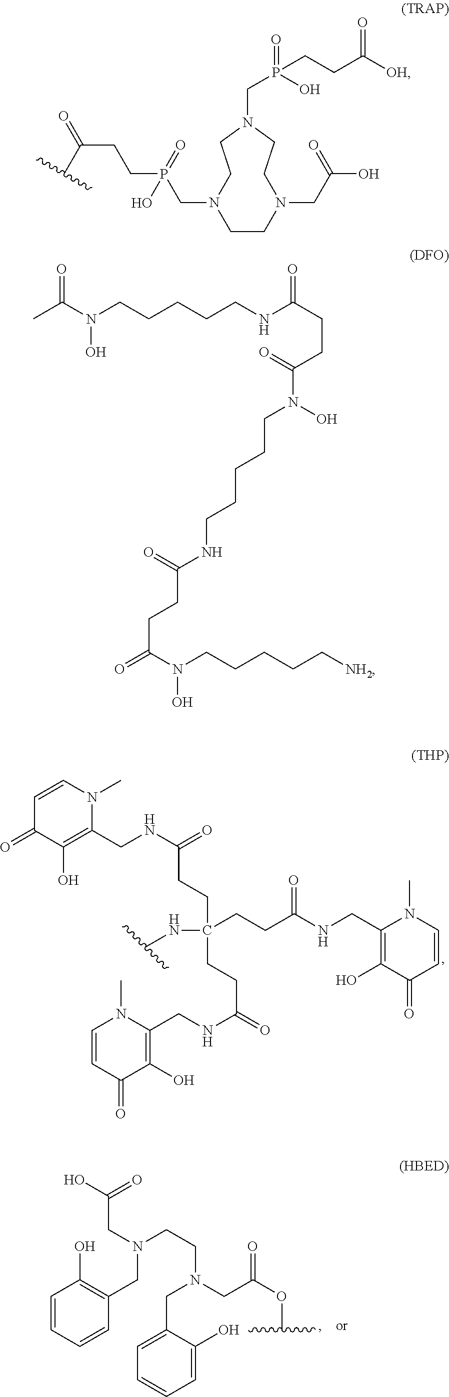 [SMILES: *C(=O)CCP(=O)(O)CN1CCN(CC(=O)O)CCN(CP(=O)(O)CCC(=O)O)CC1 ; CC(=O)N(O)CCCCCNC(=O)CCC(=O)N(O)CCCCCNC(=O)CCC(=O)N(O)CCCCCN ; *NC(CCC(=O)NCc1c(O)c(=O)ccn1C)(CCC(=O)NCc1c(O)c(=O)ccn1C)CCC(=O)NCc1c(O)c(=O)ccn1C ; *OC(=O)CN(CCN(CC(=O)O)Cc1ccccc1O)Cc1ccccc1O]

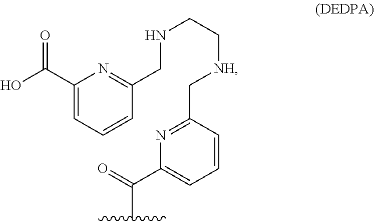 [SMILES: *C(=O)c1cccc(CNCCNCc2cccc(C(=O)O)n2)n1]

and
the radioactive metal is selected from the group consisting of ⁶⁴Cu, ⁶⁸Ga, ¹⁷⁷Lu, ⁹⁰Y, ⁸⁹Zr, ²¹¹At, ²¹²Pb, ¹⁸⁸Rh, ¹⁶⁶Ho, ²²⁵Ac, ^{99m}Tc or ¹¹¹In, ¹²³I, ¹³¹I.

R⁴, R⁵ are independently selected from the group consisting of H, halogen, —(C₁-C₆)alkyl, —(C₁-C₆)aryl, —NH₂, —N₃, —(C₁-C₆)alkynyl, —(C₆-C₁₀)aryl(C₁-C₆)alkyl, -1-halogen-1-vinyl, (C₆-C₁₀)heteroaryl(C₁-C₆)alkyl-, —(C₆-C₁₀)heteroaryl, —C(O)(C₆-C₁₀)aryl-, —OR⁶, —SR⁶, —NHR⁶, —NR⁶R⁷, —SIR⁶R⁷R⁸, —OC(O)R⁶, —C(O)R⁶, —COOR⁶, —CONR⁶R⁷, —OC(O)NR⁶R⁷, —NR⁶C(O)R⁷, —NR⁶COOR⁷, —NHC(NH₂)═NR⁶, —S(O)R⁶, —SO₂NR⁶R⁷, —NR⁶SO₂R⁷, —CN, and —NO₂; or
R⁶, R⁷, R⁸ are independently selected from the group consisting of H, halogen, —(C₁-C₆)alkyl, (C₁-C₆)aryl, —NH₂, —N₃, —(C₁-C₆)alkynyl, —(C₆-C₁₀)aryl(C₁-C₆)alkyl, -1-halogen-1-vinyl, —(C₁-C₆)alkyl(C₆-C₁₀)heteroaryl, —(C₆-C₁₀)heteroaryl, —C(O)(C₆-C₁₀)aryl-, —SO₃H, —OH, and —SH;
R⁹ is selected from the group consisting of halogen, (C₁-C₆)alkyl, —(C₁-C₆)aryl, —NH₂, —N₃, —(C₁-C₆)alkynyl, —(C₆-C₁₀)aryl(C₁-C₆)alkylenyl, 1-halogen-1-vinyl, —(C₆-C₁₀)heteroaryl(C₁-C₆)alkyl-, —(C₆-C₁₀)heteroaryl, —C(O)(C₆-C₁₀)aryl-, —OR⁶, —SR⁶, —NHR⁶, —NR⁶R⁷, —SiR⁶R⁷R⁸, —OC(O)R⁶, —C(O)R⁶, —COOR⁶, —CONR⁶R⁷, —OC(O)NR⁶R⁷, —NR⁶C(O)R⁷, —NR⁶COOR⁷, —NHC(NH₂)═NR⁶, —S(O)R⁶, —SO₂NR⁶R⁷, —NR⁶SO₂R⁷, —CN, and —NO₂;
wherein aryl or heteroaryl of R⁴, R⁵, R⁶, R⁷, R⁸, and R⁹ is optionally substituted with one or more substituents selected from the group consisting of —(C₁-C₆)alkyl, halogen, -trifluoromethyl, —OH, —SH, —NH₂, —SO₂NH₂, —(C₁-C₆)alkylhydroxy, —(C₁-C₆)alkoxy, —SO₃H, —COO(C₁-C₆)alkyl, —SO₃(C₁-C₆)alkyl, —C(O)(C₅-C₆)aryl;
R¹⁰ is OH or

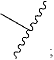

with the provisio that
if R⁷ is H, A is

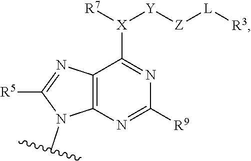

R⁵ is H, X is N, Y is CH₂, Z is phenyl,
L is —C(O)NH(CH₂)₁₋₄NH—, and R⁹ is H or Cl, then R₃ is not fluorescein;
or a pharmaceutically acceptable salt thereof.

In a second aspect, the invention is directed to a pharmaceutical composition, comprising the compound as define above and at least one pharmaceutically acceptable carrier.

In a third aspect, the invention is directed to the compound or the pharmaceutical composition as described above for use in a method of diagnosis of a disease associated with increased CD73-expression.

In a fourth aspect, the invention is directed to the compound or the pharmaceutical composition as described above for use in the treatment of a disease associated with increased CD73-expression.

In a fifth aspect, the invention is directed to the compound or the pharmaceutical composition as described above, wherein the disease is selected from the group consisting of cancer, and inflammatory diseases including autoimmune diseases.

Preferably, the cancer is selected from the group consisting of breast cancer, pancreatic cancer, colon cancer, lung cancer, kidney cancer, bladder cancer, prostate cancer, ovarian cancer, melanoma, glioma, head neck cancer and thyroid cancer or any other solid cancer.

Preferably, the inflammatory disease is selected from the group consisting of Multiple sclerosis, neuroinflammation, Parkinson's disease and rheumatoid arthritis.

### BRIEF DESCRIPTION OF THE FIGURES

FIG. 1: In vivo distribution of ¹⁸F-PSB-19427 (2) after intravenous injection in an adult C57BL/6 WT mouse. (A) Representative PET images (maximum intensity projections) acquired 0-90 minutes after injection of ¹⁸F-PSB-19427 (2). (B) Time-activity concentration curves (n=6 animals, error bars=st.dev.) of selected regions-of-interest. (C) Final ex vivo gamma counter measurements of selected tissue samples experiments after 90 min (n=3) and 4 h (260 min) (n=3).

FIG. 2: In vivo PET imaging of ¹⁸F-PSB-19427 in tumor bearing mice (s.c. MDA-MB-231 xenografts, left and right shoulder). (A) Representative PET images 4 h p.i. of ¹⁸F-PSB-19427 (2) demonstrate a pronounced accumulation of the tracer in the tumor xenografts (white arrows) that was diminished in a blocking study. Left: without blocker, right: pretreating with unlabeled compound 2 10 min before tracer injection. (B). Quantitative analysis confirms the pronounced accumulation of ¹⁸F-PSB-19427 (2) in the MDA-MB-231 tumor model as compared to muscle tissue (C). In the blocking study, quantitative analysis showed a significant decrease of tumor-to-muscle ratios (D) in animals blocked with cold substance thereby indicating in vivo binding specificity. Left: Activity concentration of [¹⁸F]2 in muscle and tumor tissues after 90 min and 260 min in unblocked mice. Right: Tumor-to-muscle ratios after 90 min and 260 min in unblocked and blocked mice.

FIG. 3: (A) Representative in vivo PET images 4 h p.i. of ¹⁸F-PSB-19427 in tumor bearing mice (s.c. AsPC-1 xenografts, left and right shoulder) demonstrate a pronounced accumulation of the tracer in the tumor xenografts (white arrows) that was diminished in a blocking study. (C) Quantitative analysis confirms the pronounced accumulation of ¹⁸F-PSB-19427 in the tumor as compared to muscle tissue. Left: no blocker. Right: Pretreating with PSB-12651 (7) 10 min before tracer injection. (B) In the blocking study, quantitative analysis showed a significant decrease of tumor-to-muscle ratios in animals blocked with PSB-12651 thereby indicating in vivo binding specificity. Left: Activity concentration of [¹⁸F]2 in muscle and tumor tissues after 90 min and 260 min in unblocked mice. Right: Tumor-to-muscle ratios after 90 min and 260 min in unblocked and blocked mice.

FIG. 4: Exemplarily-given PET images of [¹⁸F]FDG and [¹⁸F]PSB-19427 ([¹⁸F]2) comparison experiments. A. [¹⁸F]FDG-and [¹⁸F]PSB-19427 ([¹⁸F]2) labelled mice after 90 min and 4 h. B. Quantitative analysis of [¹⁸F]FDG and [¹⁸F]PSB-19427 ([¹⁸F]2) uptake in the MDA-MB-231 tumors and resulting tumor-to-muscle ratios of [¹⁸F]FDG and [¹⁸F]2. Quantitative analysis confirms the higher accumulation of ¹⁸F-PSB-19427 in the tumor as compared to FDG-, especially in relation to muscle uptake (B).

FIG. 5: External calibration of reference compounds.

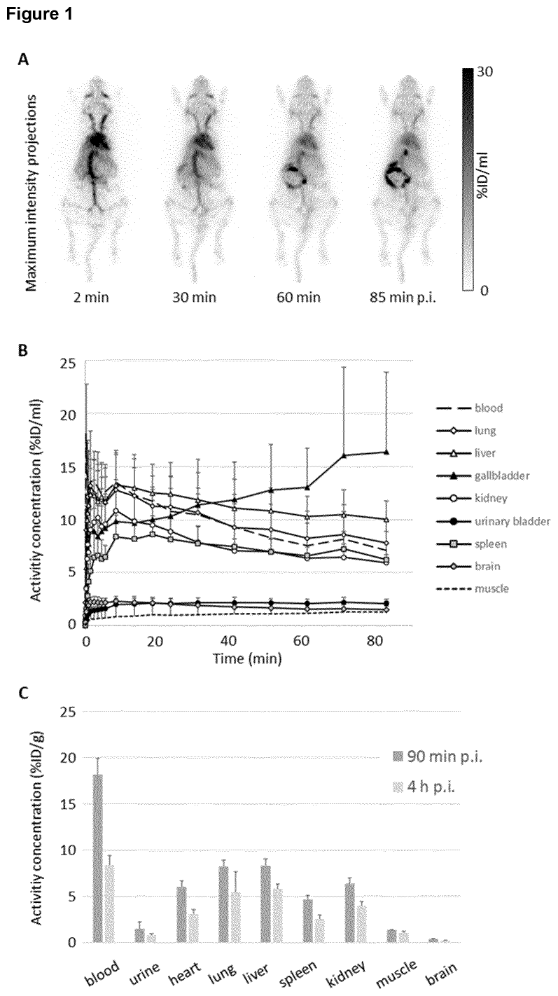

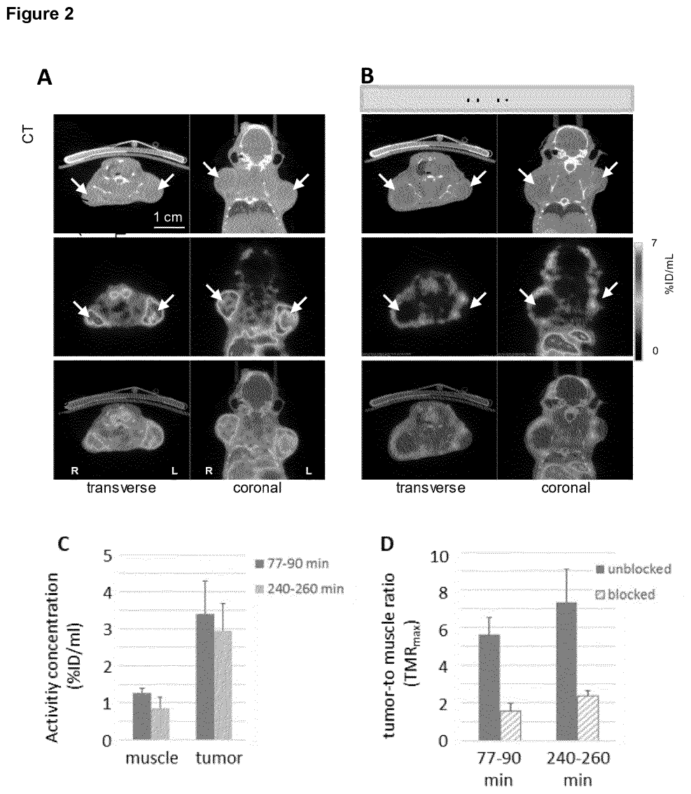

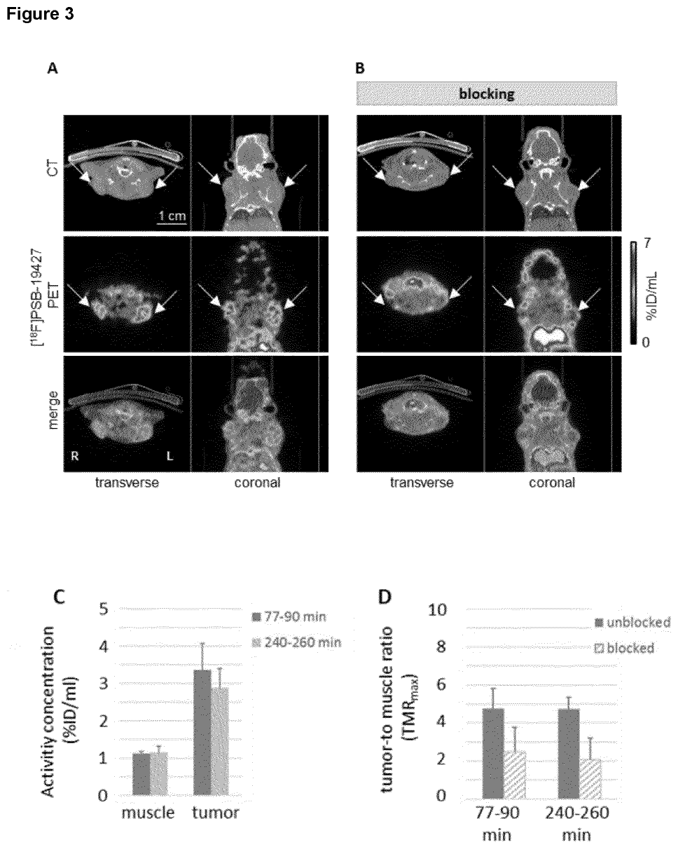

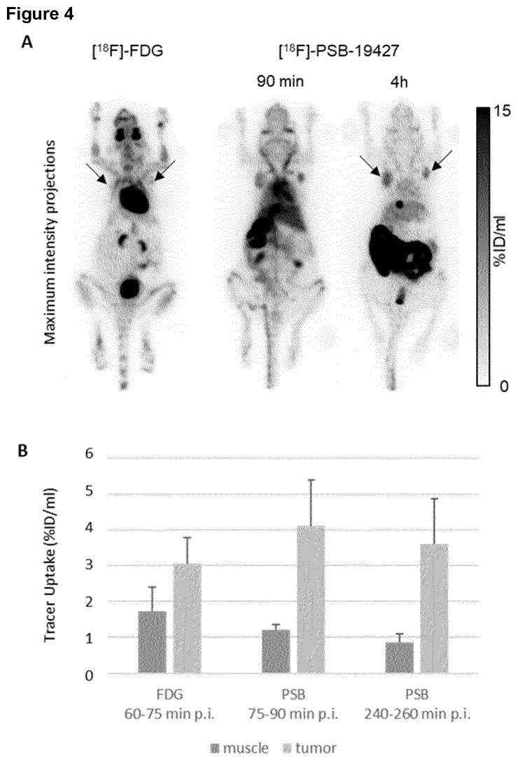

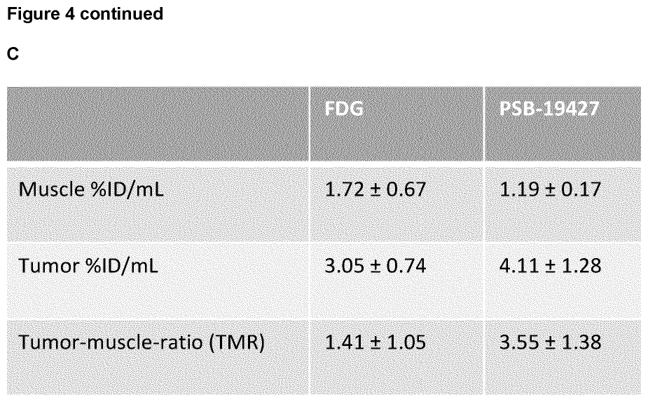

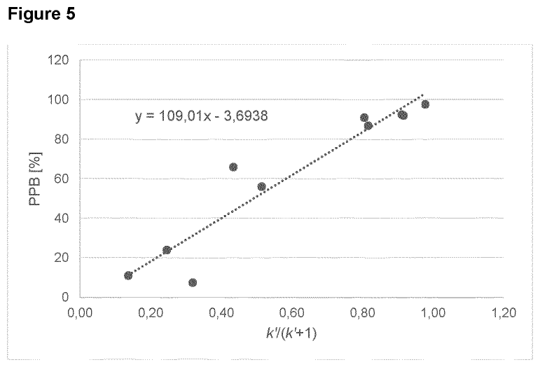

### DETAILED DESCRIPTION OF THE INVENTION

### Definitions

The term “alkyl” refers to a monoradical of a saturated straight or branched hydrocarbon. Preferably, the alkyl group comprises from 1 to 10 carbon atoms, i.e., 1, 2, 3, 4, 5, or 6 carbon atoms, more preferably 1 to 8 carbon atoms, such as 1 to 6 or 1 to 4 carbon atoms. Exemplary alkyl groups include methyl, ethyl, propyl, iso-propyl, butyl, iso-butyl, tert-butyl, n-pentyl, iso-pentyl, sec-pentyl, neo-pentyl, 1,2-dimethyl-propyl, iso-amyl, n-hexyl, iso-hexyl, sec-hexyl, n-heptyl, iso-heptyl, n-octyl, 2-ethyl-hexyl, n-nonyl, n-decyl, n-undecyl, n-dodecyl, and the like.

The term “aryl” refers to a monoradical or a diradical of an aromatic cyclic hydrocarbon in the context of the present invention. For example, for illustration, in general formula (I), Y and Z may be aryl, wherein the aryl is a diradical. Preferably, the aryl group contains 5 to 14 carbon atoms which can be arranged in one ring (e.g., phenyl) or two or more condensed rings (e.g., naphthyl). Exemplary aryl groups include cyclopropenylium, cyclopentadienyl, phenyl, indenyl, naphthyl, azulenyl, fluorenyl, anthryl, and phenanthryl. Preferably, “aryl” refers to a monocyclic ring containing 6 carbon atoms or an aromatic bicyclic ring system containing 10 carbon atoms. Preferred examples are phenyl and naphthyl.

The term “heteroaryl” means an aryl group as defined above in which one or more carbon atoms in the aryl group are replaced by heteroatoms of O, S, or N. Preferably, heteroaryl refers to a five or six-membered aromatic monocyclic ring wherein 1, 2, or 3 carbon atoms are replaced by the same or different heteroatoms of O, N, or S. Alternatively, it means an aromatic bicyclic or tricyclic ring system wherein 1, 2, 3, 4, or 5 carbon atoms are replaced with the same or different heteroatoms of O, N, or S. For example, as illustration, “—(C₆-C₁₀)heteroaryl” refers to an aromatic ring with 6 to 10 carbon atoms in the ring, wherein one or more carbon atoms in the aryl group are replaced by heteroatoms of O, S, or N. Preferably, in each ring of the heteroaryl group the maximum number of O atoms is 1, the maximum number of S atoms is 1, and the maximum total number of O and S atoms is 2. Exemplary heteroaryl groups include furanyl, thienyl, oxazolyl, isoxazolyl, oxadiazolyl (1,2,5- and 1,2,3-), pyrrolyl, imidazolyl, pyrazolyl, triazolyl (1,2,3- and 1,2,4-), tetriazolyl, thiazolyl, isothiazolyl, thiadiazolyl (1,2,3- and 1,2,5-), pyridyl, pyrimidinyl, pyrazinyl, triazinyl (1,2,3-, 1,2,4-, and 1,3,5-), benzofuranyl (1- and 2-), indolyl, isoindolyl, benzothienyl (1- and 2-), 1H-indazolyl, benzimidazolyl, benzoxazolyl, indoxazinyl, benzisoxazolyl, benzothiazolyl, benzisothiazolyl, benzotriazolyl, quinolinyl, isoquinolinyl, benzodiazinyl, quinoxalinyl, quinazolinyl, benzotriazinyl (1,2,3- and 1,2,4-benzotriazinyl), pyridazinyl, phenoxazinyl, thiazolopyridinyl, pyrrolothiazolyl, phenothiazinyl, isobenzofuranyl, chromenyl, xanthenyl, phenoxathiinyl, pyrrolizinyl, indolizinyl, indazolyl, purinyl, quinolizinyl, phthalazinyl, naphthyridinyl (1,5-, 1,6-, 1,7-, 1,8-, and 2,6-), cinnolinyl, pteridinyl, carbazolyl, phenanthridinyl, acridinyl, perimidinyl, phenanthrolinyl (1,7-, 1,8-, 1,10-, 3,8-, and 4,7-), phenazinyl, oxazolopyridinyl, isoxazolopyridinyl, pyrrolooxazolyl, and pyrrolopyrrolyl. Exemplary 5- or 6-memered heteroaryl groups include furanyl, thienyl, oxazolyl, isoxazolyl, oxadiazolyl (1,2,5- and 1,2,3-), pyrrolyl, imidazolyl, pyrazolyl, triazolyl (1,2,3- and 1,2,4-), thiazolyl, isothiazolyl, thiadiazolyl (1,2,3- and 1,2,5-), pyridyl, pyrimidinyl, pyrazinyl, triazinyl (1,2,3-, 1,2,4-, and 1,3,5-), and pyridazinyl.

The term “alkynyl” refers to a monoradical or a diradical of an unsaturated straight or branched hydrocarbon having at least one carbon-carbon triple bond. For example, for illustration, in general formula (I), L may be alkynyl, wherein alkynyl is a diradical. Generally, the maximal number of carbon-carbon triple bonds in the alkynyl group can be equal to the integer which is calculated by dividing the number of carbon atoms in the alkynyl group by 2 and, if the number of carbon atoms in the alkynyl group is uneven, rounding the result of the division down to the next integer. For example, for an alkynyl group having 9 carbon atoms, the maximum number of carbon-carbon triple bonds is 4. Preferably, the alkynyl group has 1 to 4, i.e., 1, 2, 3, or 4, more preferably 1 or 2 carbon-carbon triple bonds. Preferably, the alkynyl group comprises from 2 to 10 carbon atoms, i.e., 2, 3, 4, 5, 6, 7, 8, 9, or 10 carbon atoms, more preferably 2 to 8 carbon atoms, such as 2 to 6 carbon atoms or 2 to 4 carbon atoms. Thus, in a preferred embodiment, the alkynyl group comprises from 2 to 10 carbon atoms and 1, 2, 3, 4, or 5 (preferably 1, 2, or 3) carbon-carbon triple bonds, more preferably it comprises 2 to 8 carbon atoms and 1, 2, 3, or 4 (preferably 1 or 2) carbon-carbon triple bonds, such as 2 to 6 carbon atoms and 1, 2 or 3 carbon-carbon triple bonds or 2 to 4 carbon atoms and 1 or 2 carbon-carbon triple bonds. Exemplary alkynyl groups include ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, 5-hexynyl, 1-heptynyl, 2-heptynyl, 3-heptynyl, 4-heptynyl, 5-heptynyl, 6-heptynyl, 1-octynyl, 2-octynyl, 3-octynyl, 4-octynyl, 5-octynyl, 6-octynyl, 7-octynyl, 1-nonylyl, 2-nonynyl, 3-nonynyl, 4-nonynyl, 5-nonynyl, 6-nonynyl, 7-nonynyl, 8-nonynyl, 1-decynyl, 2-decynyl, 3-decynyl, 4-decynyl, 5-decynyl, 6-decynyl, 7-decynyl, 8-decynyl, 9-decynyl, and the like. If an alkynyl group is attached to a nitrogen atom, the triple bond cannot be alpha to the nitrogen atom.

The term “halogen” means fluoro, chloro, bromo, or iodo, preferably fluoro. In one embodiment, the halogen is fluoro, bromo, or iodo. In another embodiment, the halogen is bromo.

“A pharmaceutically acceptable salt” is intended to mean a salt that retains the biological effectiveness of the free acids and bases of the specified compound and that is not biologically or otherwise undesirable. A compound of the invention may possess a sufficiently acidic, a sufficiently basic, or both functional groups, and accordingly react with any of a number of inorganic or organic bases, and inorganic and organic acids, to form a pharmaceutically acceptable salt. Exemplary pharmaceutically acceptable salts include those salts prepared by reaction of the compounds of the present invention with a mineral or organic acid or an inorganic base, such as salts including sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, 7-hydroxybutyrates, glycolates, tartrates, methane-sulfonates, propanesulfonates, naphthalene-1-sulfonates, naphthalene-2-sulfonates, and mandelates.

The term “analogue”, as used herein, means a member of a group of chemical compounds which have a core structure in common and which exhibit a common effect but which differ in respect to their elemental composition. In particular, an analogue is a compound having a core structure identical to that of another compound, but differing from it in respect of one or more atoms, functional groups, or substructures and retaining the common property.

### Compounds

The invention is directed to a compound according to general formula (I)

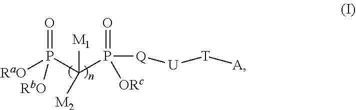

wherein
R^{a}, R^{b} and R^{c} are independently selected from the group consisting of H, —(C₁-C₆)alkyl, —(C₆-C₁₀)aryl, —C(O)(C₁-C₆)alkyl, —(C₁-C₆)alkyl(C₆-C₁₀)heteroaryl, —(C₆-C₁₀)heteroaryl and —C(O)aryl, preferably H;
M₁ and M₂ are independently selected from the group consisting of H, —OH and halogen, preferably H;
n is 1 to 6, preferably 1 to 3, more preferably 1;
Q is selected from the group consisting of O, S, CH₂, NH, preferably O;
U is selected from the group consisting of O, S, CH₂, (CH₂)₂, NH, preferably CH₂;
T is selected from the group consisting of

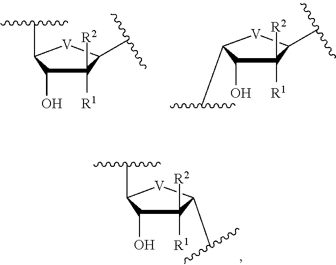

preferably

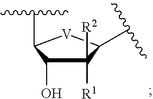

V is selected from the group consisting of O, NH, S, CH₂; preferably O
R¹ and R² are independently selected from the group consisting of H, OH, SH, —O(C₁-C₆)alkyl, —S(C₁-C₆)alkyl, —NH₂, —NH(C₁-C₆)alkyl, —N₃ and halogen, preferably H and —OH;
In one embodiment R¹ is OH and R² is H.

A is selected from the group consisting of

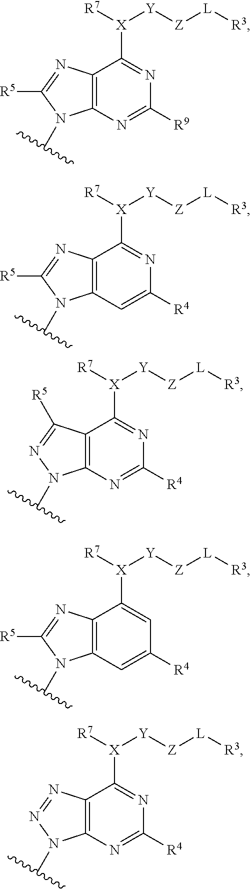

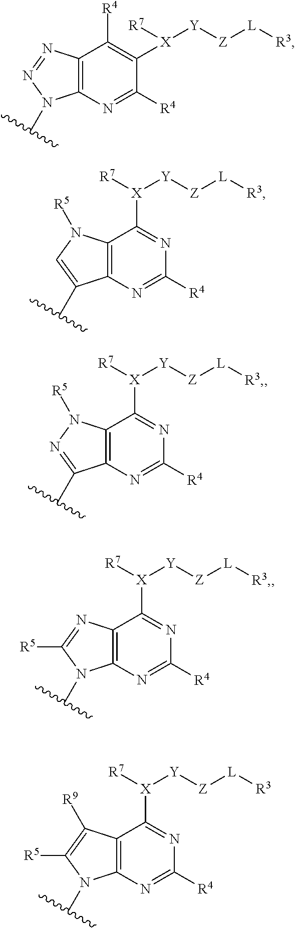

preferably

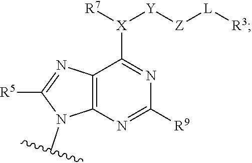

X is selected from the group consisting of O, S, N,

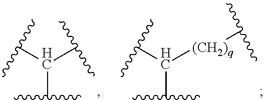

preferably N
q is 0 to 6, preferably 0 to 4, more preferably 0
Y is selected from the group consisting of —(C₆-C₁₀)aryl-, —(CH₂)ₚ—, —(CH₂)ₚC(O)—, —(C₆-C₁₀)heteroaryl(CH₂)ₚ—, —(C₆-C₁₀)heteroaryl-, —(CH₂)ₚ(C₆-C₁₀)aryl-, and —(C₆-C₁₀)arylC(O)—, preferably —(CH₂)ₚ—, or —(C₆-C₁₀)arylC(O)—, more preferably —(CH₂)ₚ—
Z is selected from the group consisting of

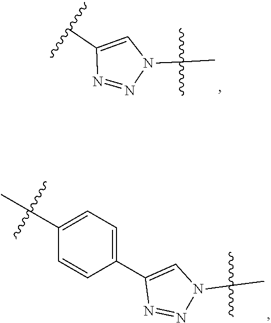 [SMILES: *c1cn(*)nn1 ; *c1ccc(-c2cn(*)nn2)cc1]

H, NH, O, S,

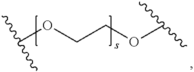

—(CH₂)ₚ—, —(C₆-C₁₀)aryl, —(C₆-C₁₀)aryl(CH₂)p, —C(O)(CH₂)ₚ—, —(C₆-C₁₀)heteroaryl(CH₂)ₚ—, —(C₆-C₁₀)heteroaryl-, —(C₆-C₁₀)arylC(O)—, preferably

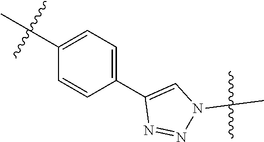 [SMILES: *c1ccc(-c2cn(*)nn2)cc1]

and —(C₆-C₁₀)arylC(O)—, wherein optionally the aryl groups may be substituted by one or more substituents selected from the group consisting of —(C₁-C₆)alkyl, —(C₂-C₆)alkynyl, -halogen, -trifluoromethyl, —OH, —SH, —NH₂, —SO₂NH₂, —(C₁-C₆)alkylOH, —O(C₁-C₆)alkyl, —SO₃H, —(CH₂)₁₋₆COOH, —COOH, —C(O)NH₂, —SO₃(C₁-C₆)alkyl, (C₅-C₆)arylCH₂C(O)—, —C(O)NH(CH₂)ₒNH₂;
o is an integer from 1 to 8, preferably 1 to 4, most preferably 4;
p is an integer from 1 to 6, preferably 1 to 4, most preferably 4;
L comprises L¹ and L², with L¹ connected to L², L¹-L²
L¹ is selected from the group consisting of absent, —(CH₂)_{q}—, —C(O)NH(CH₂)ₚNH—, —(CH₂)_{q}(C₅-C₁₀)aryl-, —(C₁-C₁₀)alkynyl-, —(C₆-C₁₀)aryl(CH₂)ₚ—, 1-halo-1-vinyl, —(C₆-C₁₀)heteroarylenyl(CH₂)ₚ—, —(C₆-C₁₀)heteroaryl-, and —(C₆-C₁₀)arylC(O)—,

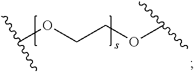

preferably L is absent, or —C(O)NH(CH₂)₁₋₄NH—;
s is an integer from 1 to 60, preferably 1 to 50, more preferably 2 to 30;
q is an integer from 1 to 10, preferably 1 to 6, most preferably 5;
L² is selected from the group consisting of absent,

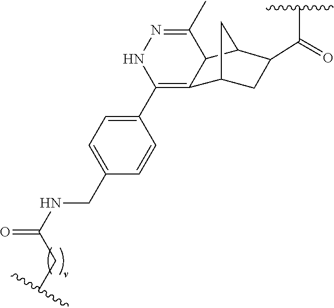

v, is 1 to 9, preferably 1 to 7;
v may be 1, 2, 3, 4, 5, 6 or 7, preferably 1, 4 or 7,

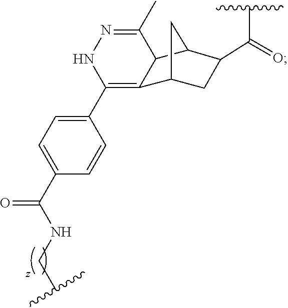

z is 1 to 6, preferably 1 to 5, more preferably 5;
R³ is selected from the group consisting of
i)

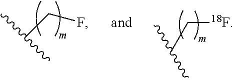

m is an integer of from 2 to 10; or
ii),

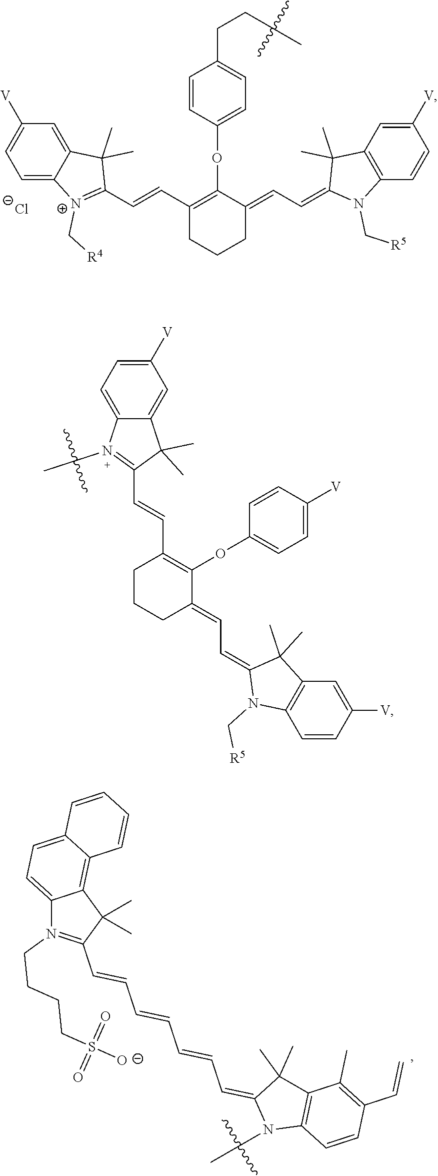

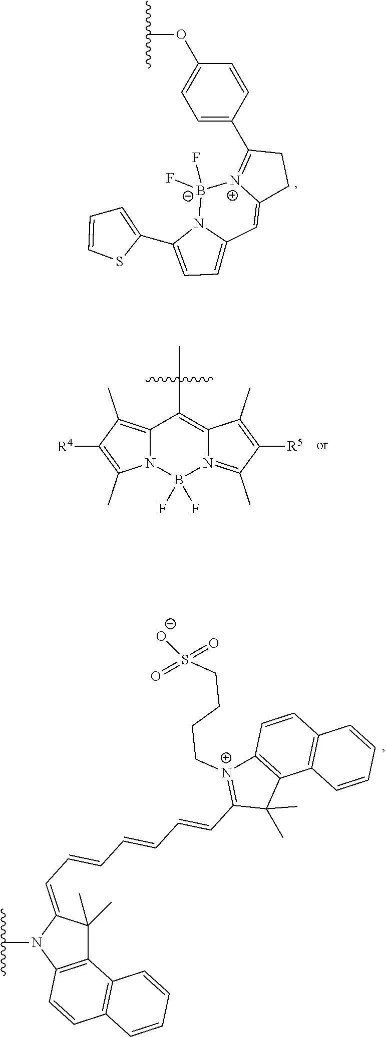

iii) a fluorophore moiety selected from the group consisting of FITC, Fluorescein, NBD, Dansyl, Squaraine Rotaxane, Bodipy FL, Bodipy TR, Bodipy 630/650 X, Bodipy 650/655 X, Texas Red, Cy5, 1-pyrene, EVOBlue 30, Alexa Fluor 532, Alexa Fluor 488-5, 488-6, or mixture thereof, Tamra, Tamra 5/6-X-SE, Alexa Fluor 488 azide 5 isomer, Alexa Fluor 488 5 isomer, Alexa Fluor 488 5/6 mixed isomers, NIR dye 700, NIR dye 800, Janelia Fluor 549 amide, Janelia Fluor 646 amide, and derivates, analogs and related fluorophores thereof; or
iv) a chelating moiety binding a radioactive metal,
wherein the chelating moiety is selected from the group consisting of

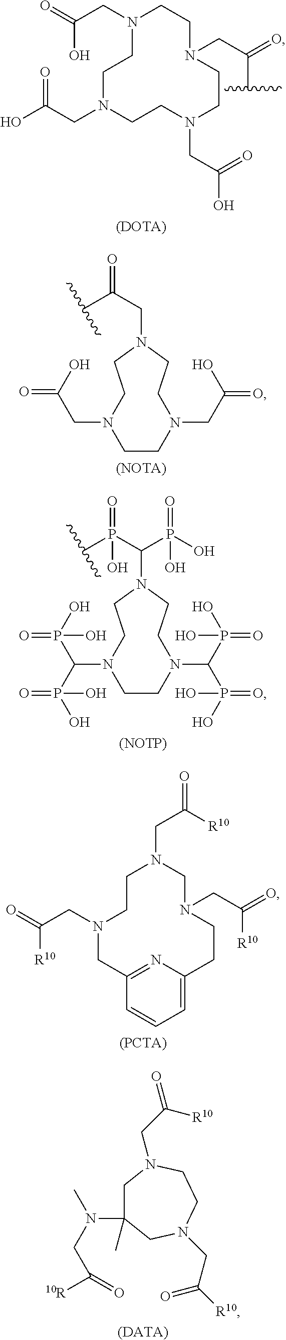

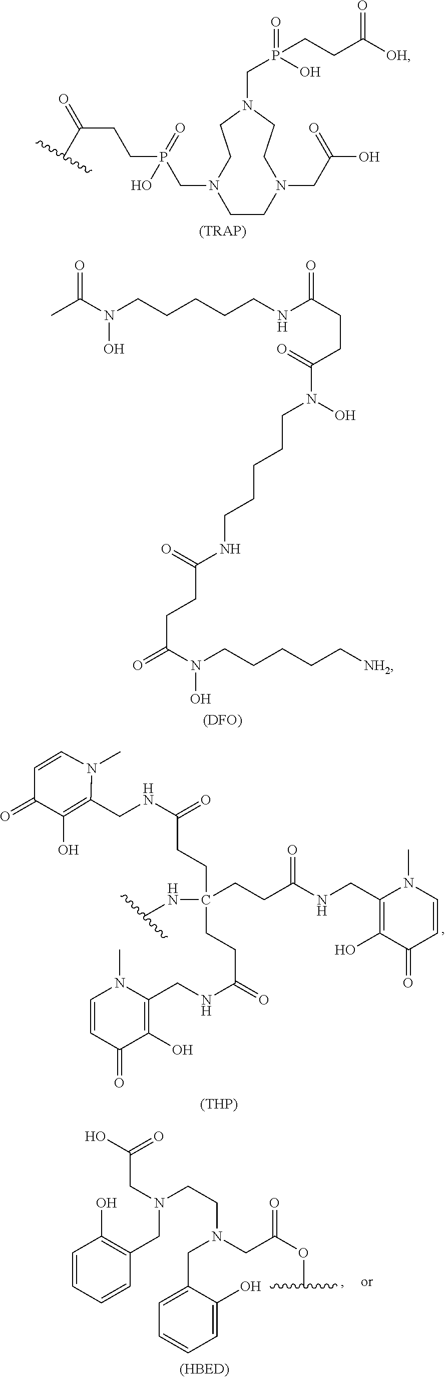

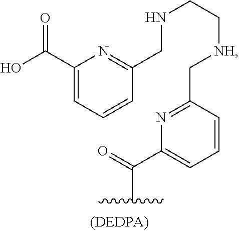 [SMILES: *C(=O)c1cccc(CNCCNCc2cccc(C(=O)O)n2)n1]

and
the radioactive metal is selected from the group consisting of ⁶⁴Cu, ³⁸Ga, ¹⁷⁷Lu, ⁹⁰Y, ⁸⁹Zr, ²¹¹At, ²¹²Pb, ¹⁸⁸Rh, ¹⁶⁶Ho, ²²⁵Ac, ^{99m}Tc or ¹¹¹In, ¹²³I, ¹³¹I;
preferably, R³ is selected from the group consisting of CH₂—F¹⁸,

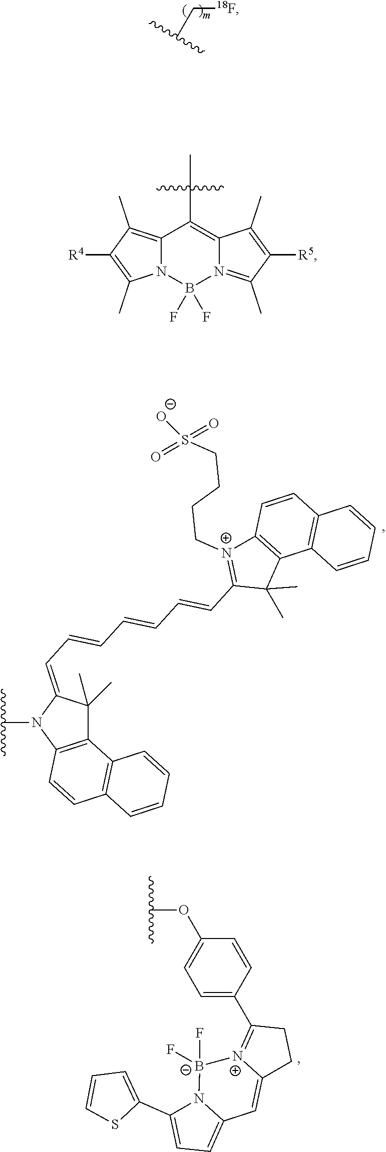

m is 2 to 4, preferably 2.
R⁴, R⁵ are independently selected from the group consisting of H, halogen, —(C₁-C₆)alkyl, —(C₁-C₆)aryl, —NH₂, —N₃, —(C₁-C₆)alkynyl, —(C₆-C₁₀)aryl(C₁-C₆)alkyl, -1-halogen-1-vinyl, (C₆-C₁₀)heteroaryl(C₁-C₆)alkyl-, —(C₆-C₁₀)heteroaryl, —C(O)(C₆-C₁₀)aryl-, —OR⁶, —SR⁶, —NHR⁶, —NR⁶R⁷, —SiR⁶R⁷R⁸, —OC(O)R⁶, —C(O)R⁶, —COOR⁶, —CONR⁶R⁷, —OC(O)NR⁶R⁷, —NR⁶C(O)R⁷, —NR⁶COOR⁷, —NHC(NH₂)═NR⁶, —S(O)R⁶, —SO₂NR⁶R⁷, —NR⁶SO₂R⁷, —CN, and —NO₂, preferably H, —(C₁-C₆)alkyl or halogen, preferably H or halogen, more preferably halogen;
R⁶, R⁷, R⁸ are independently selected from the group consisting of H, halogen, —(C₁-C₆)alkyl, (C₁-C₆)aryl, —NH₂, —N₃, —(C₁-C₆)alkynyl, —(C₆-C₁₀)aryl(C₁-C₆)alkyl, -1-halogen-1-vinyl, —(C₁-C₆)alkyl(C₆-C₁₀)heteroaryl, —(C₆-C₁₀)heteroaryl, —C(O)(C₆-C₁₀)aryl-, —SO₃H, —OH, and —SH;
preferably R⁶ is H, —(C₁-C₆)alkyl, —(C₆-C₁₀)aryl(C₁-C₆)alkyl, —(C₁-C₆)alkyl(C₆-C₁₀)heteroaryl, preferably R⁷ is H or —(C₁-C₆)alkyl, preferably H or (C₂-C₄)alkyl; more preferably (C₃)alkyl, Preferably R⁸ is H or —(C₁-C₆)alkyl;
X is N; and/or
Y is —(C₁-C₆)alkyl-; and/or
R⁹ is selected from the group consisting of halogen, (C₁-C₆)alkyl, —(C₁-C₆)aryl, —NH₂, —N₃, —(C₁-C₆)alkynyl, —(C₆-C₁₀)aryl(C₁-C₆)alkylenyl, 1-halogen-1-vinyl, —(C₆-C₁₀)heteroaryl(C₁-C₆)alkyl-, —(C₆-C₁₀)heteroaryl, —C(O)(C₆-C₁₀)aryl-, —OR⁶, —SR⁶, —NHR⁶, —NR⁶R⁷, —SiR⁶R⁷R⁸, —OC(O)R⁶, —C(O)R⁶, —COOR⁶, —CONR⁶R⁷, —OC(O)NR⁶R⁷, —NR⁶C(O)R⁷, —NR⁶COOR⁷, —NHC(NH₂)—NR⁶, —S(O)R⁶, —SO₂NR⁶R⁷, —NR⁶SO₂R⁷, —CN, and —NO₂, preferably H, halogen, or —(C₁-C₆)alkyl, more preferably H or halogen;
wherein aryl or heteroaryl of R⁴, R⁵, R⁶, R⁷, R⁸, and R⁹ is optionally substituted with one or more substituents selected from the group consisting of —(C₁-C₆)alkyl, halogen, -trifluoromethyl, —OH, —SH, —NH₂, —SO₂NH₂, —(C₁-C₆)alkylhydroxy, —(C₁-C₆)alkoxy, —SO₃H, —COO(C₁-C₆)alkyl, —SO₃(C₁-C₆)alkyl, —C(O)(C₅-C₆)aryl;
R¹⁰ is OH or

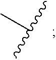

with the provisio that
if R⁷ is H, A is

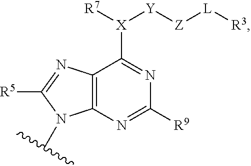

R⁵ is H, X is N, Y is CH₂, Z is phenyl, L is —C(O)NH(CH₂)₁₋₄NH—, and R⁹ is H or Cl, then R₃ is not fluorescein;
or a pharmaceutically acceptable salt thereof.

In one embodiment in formula (I), R^{a}, R^{b} and R^{c} are H; and/or
M₁ and M₂ are H; and/or
n is 1; and/or
Q is O; and/or
U is CH₂; and/or
R¹ is —OH; and/or
R² is H; and/or
A is

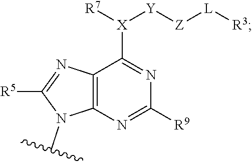

and/or
R⁴ is H, —(C₁-C₆)alkyl or halogen, preferably H or halogen, more preferably halogen; and/or
R⁵ is H and/or
R⁶ is H, —(C₁-C₆)alkyl, —(C₆-C₁₀)aryl(C₁-C₆)alkyl, —(C₁-C₆)alkyl(C₆-C₁₀)heteroaryl; and/or
R⁷ is H or —(C₁-C₆)alkyl, preferably H or (C₂-C₄)alkyl; more preferably (C₃)alkyl and/or
R⁸ is H or —(C₁-C₆)alkyl; and/or
R⁹ is H, halogen, or —(C₁-C₆)alkyl, preferably H or halogen; and/or
X is N; and/or
Y is —(CH₂)ₚ— and/or
Z is

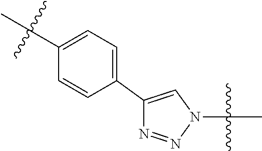 [SMILES: *c1ccc(-c2cn(*)nn2)cc1]

or —(C₆-C₁₀)arylC(O); and/or
L is absent, or —C(O)NH(CH₂)₁₋₄NH—; and/or
R³ is

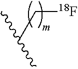

or Fluorescein.

In one embodiment, the compound is

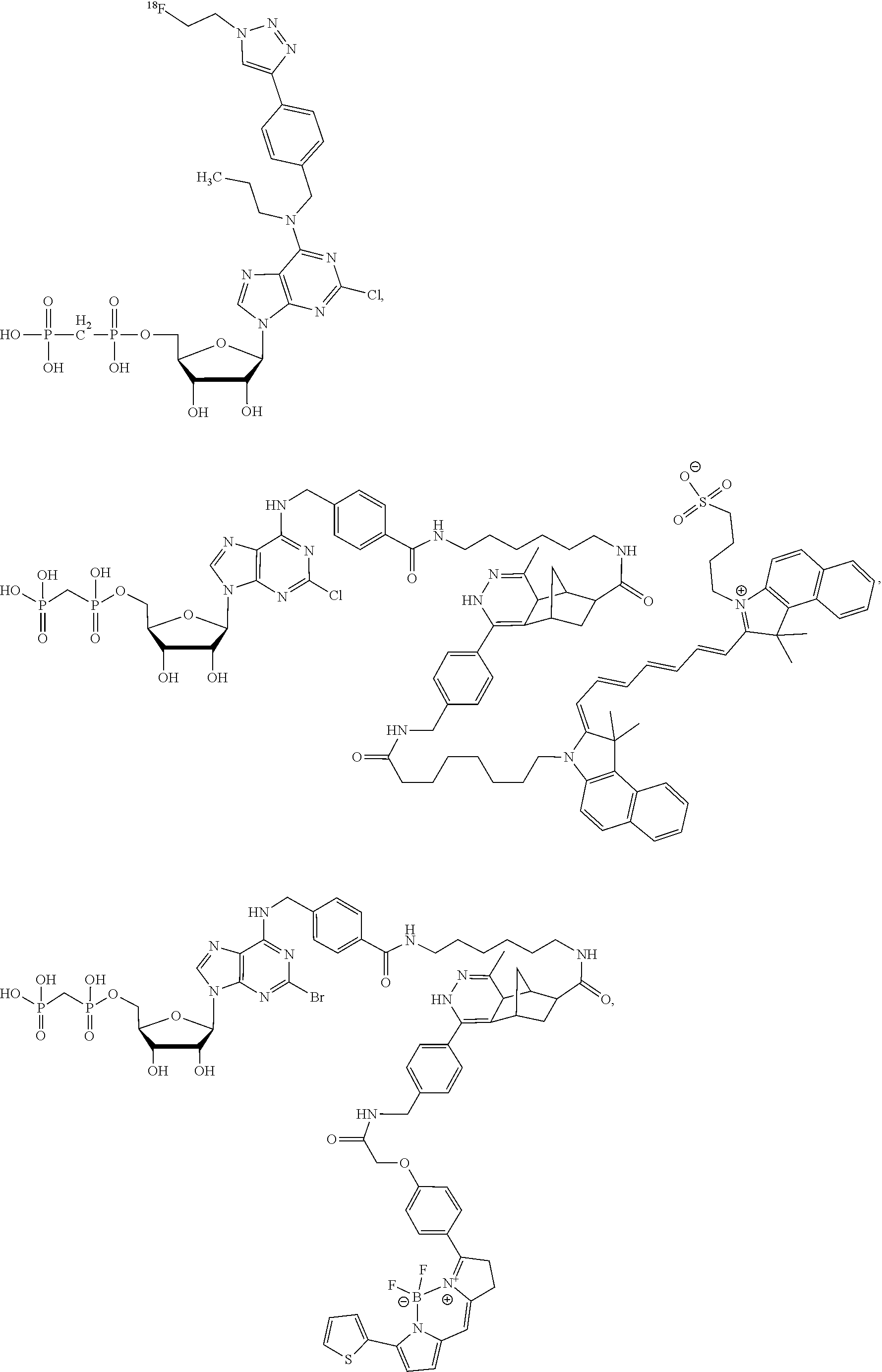

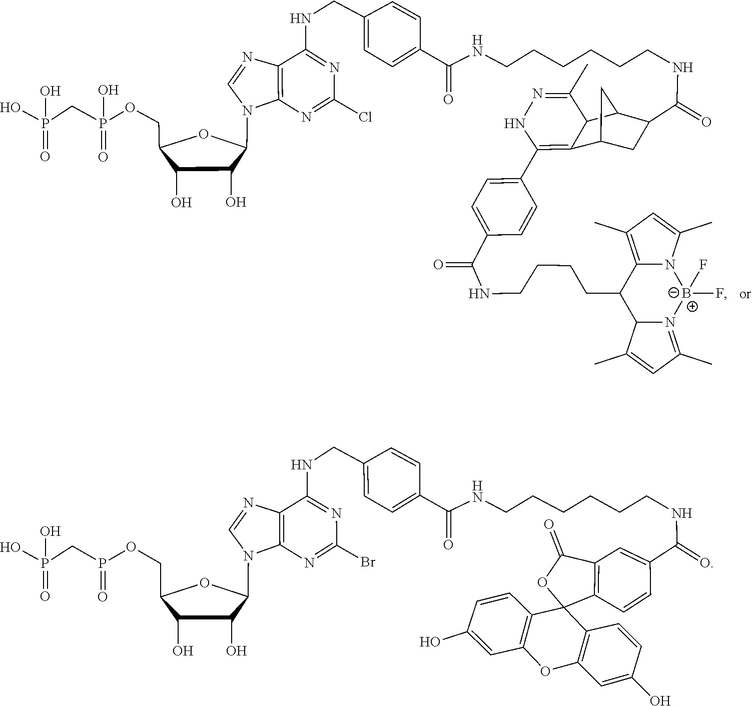

In a further embodiment
V is O;
Q is O;
U is CH₂; A is

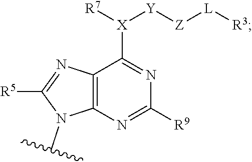

X is N;
Y is CH₂;
Z is phenyl or

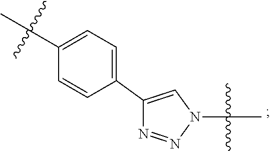 [SMILES: *c1ccc(-c2cn(*)nn2)cc1]

L is absent or —C(O)NH(CH₂)ₚNH—;
p in —C(O)NH(CH₂)ₚNH— is 1 to 6, preferably 4 to 6, most preferably 6;
R³ is CH₂—F¹⁸,

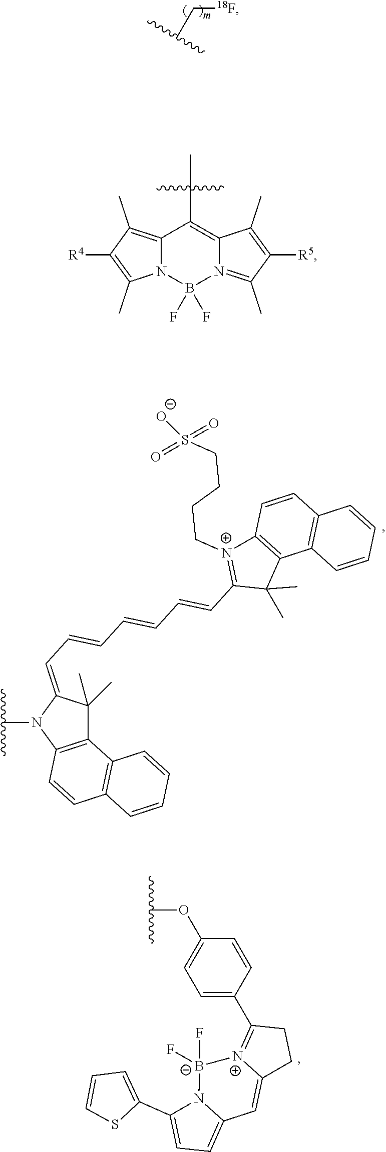

m is 2 to 4, preferably 2.

### Pharmaceutical Compositions

The invention is further directed to a pharmaceutical composition, comprising the compound of as described above and at least one pharmaceutically acceptable carrier.

“Carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, including but not limited to peanut oil, soybean oil, mineral oil, sesame oil and the like. Water is a preferred carrier when the pharmaceutical composition is administered orally. Saline and aqueous dextrose are preferred carriers when the pharmaceutical composition is administered intravenously. Saline solutions and aqueous dextrose and glycerol solutions are preferably employed as liquid carriers for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsions, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the therapeutic, preferably in purified form, together with a suitable amount of carrier so as to provide the form for proper administration to the patient. The formulation should suit the mode of administration.

Actual dosage levels of the compound in the pharmaceutical compositions of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present invention employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

Generally, out of 100% (for the pharmaceutical formulations/compositions), the amount of active ingredient (in particular, the amount of the compound of the present invention, optionally together with other therapeutically active agents, if present in the pharmaceutical formulations/compositions) will range from about 0.01% to about 99%, preferably from about 0.1% to about 70%, most preferably from about 1% to about 30%, wherein the reminder is preferably composed of the one or more pharmaceutically acceptable excipients.

The amount of active ingredient, e.g., a compound of the invention, in a unit dosage form and/or when administered to an indiviual or used in therapy, may range from about 0.1 mg to about 1000 mg (for example, from about 1 mg to about 500 mg, such as from about 10 mg to about 200 mg) per unit, administration or therapy. In certain embodiments, a suitable amount of such active ingredient may be calculated using the mass or body surface area of the individual, including amounts of between about 1 mg/Kg and 10 mg/Kg (such as between about 2 mg/Kg and 5 mg/Kg), or between about 1 mg/m² and about 400 mg/m² (such as between about 3 mg/m² and about 350 mg/m² or between about 10 mg/m² and about 200 mg/m²).

For the therapeutic/pharmaceutical formulations, compositions of the present invention include those suitable for enteral administration (such as oral or rectal) or parenteral administration (such as nasal, topical (including vaginal, buccal and sublingual)). The compositions may conveniently be presented in unit dosage form and may be prepared by any methods known in the art of pharmacy. The amount of active ingredient (in particular, the amount of a compound of the present invention) which can be combined with a carrier material to produce a pharmaceutical composition (such as a single dosage form) will vary depending upon the individual being treated, and the particular mode of administration. The amount of active ingredient which can be combined with a carrier material to produce a single dosage form will generally be that amount of the composition which produces a therapeutic effect.

The expressions “enteral administration” and “administered enterally” as used herein mean that the drug administered is taken up by the stomach and/or the intestine. Examples of enteral administration include oral and rectal administration. The expressions “parenteral administration” and “administered parenterally” as used herein mean modes of administration other than enteral administration, usually by injection or topical application, and include, without limitation, intravenous, intramuscular, intraarterial, intrathecal, intracapsular, intraosseous, intraorbital, intracardiac, intradermal, intraperitoneal, transtracheal, subcutaneous, subcuticular, intraarticular, subcapsular, intracerebral, intracerebroventricular, subarachnoid, intraspinal, epidural and intrasternal administration (such as by injection and/or infusion) as well as topical administration (e.g., epicutaneous, inhalational, or through mucous membranes (such as buccal, sublingual or vaginal)).

For oral administration, the pharmaceutical composition of the invention can take the form of, for example, tablets or capsules prepared by conventional means with pharmaceutical acceptable excipients such as binding agents (e.g., pregelatinised maize starch, polyvinylpyrrolidone, hydroxypropyl methylcellulose), fillers (e.g., lactose, microcrystalline cellulose, calcium hydrogen phosphate), lubricants (e.g., magnesium stearate, talc, silica), disintegrants (e.g., potato starch, sodium starch glycolate), or wetting agents (e.g., sodium lauryl sulphate). Liquid preparations for oral administration can be in the form of, for example, solutions, syrups, or suspensions, or can be presented as a dry product for constitution with water or other suitable vehicle before use. Such liquid preparation can be prepared by conventional means with pharmaceutically acceptable additives such as suspending agents (e.g., sorbitol, syrup, cellulose derivatives, hydrogenated edible fats), emulsifying agents (e.g., lecithin, acacia), non-aqueous vehicles (e.g., almond oil, oily esters, ethyl alcohol, fractionated vegetable oils), preservatives (e.g., methyl or propyl-p-hydroxycarbonates, sorbic acids). The preparations can also contain buffer salts, flavouring, coloring and sweetening agents as deemed appropriate. Preparations for oral administration can be suitably formulated to give controlled release of the pharmaceutical composition of the invention.

The pharmaceutical composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc.

For administration by inhalation, the pharmaceutical composition of the invention is conveniently delivered in the form of an aerosol spray presentation from a pressurised pack or a nebulizer, with the use of a suitable propellant (e.g., dichlorodifluoromethane, trichlorofluoromethane, dichlorotetrafluoroethane, carbon dioxide, nitrogen, or other suitable gas). In the case of a pressurised aerosol, the dosage unit can be determined by providing a valve to deliver a metered amount. Capsules and cartridges of, for example, gelatine, for use in an inhaler or insufflator can be formulated containing a powder mix of the pharmaceutical composition of the invention and a suitable powder base such as lactose or starch.

The pharmaceutical composition of the invention can be formulated for parenteral administration by injection, for example, by bolus injection or continuous infusion. Formulations for injection can be presented in units dosage form (e.g., in phial, in multi-dose container), and with an added preservative. The pharmaceutical composition of the invention can take such forms as suspensions, solutions or emulsions in oily or aqueous vehicles, and can contain formulatory agents such as suspending, stabilizing, or dispersing agents. Alternatively, the agent can be in powder form for constitution with a suitable vehicle (e.g., sterile pyrogen-free water) before use. Typically, compositions for intravenous administration are solutions in sterile isotonic aqueous buffer. Where necessary, the composition can also include a solubilizing agent and a local anesthetic such as lignocaine to ease pain at the site of the injection. Generally, the ingredients are supplied either separately or mixed together in unit dosage form, for example, as a dry lyophilised powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients can be mixed prior to administration.

### Medical Applications

The invention is further directed to the compound or the pharmaceutical composition as described above for use in a method of diagnosis of a disease associated with increased CD73-expression.

The invention is further directed to the compound or the pharmaceutical for use in the treatment of a disease associated with increased CD73-expression.

The disease may be selected from the group consisting of cancer, and inflammatory diseases.

The cancer is preferably a solid tumor, more preferably a cancer selected from the group consisting of breast cancer, pancreatic cancer, colon cancer, lung cancer, kidney cancer, bladder cancer, prostate cancer, ovarian cancer, melanoma, glioma, head neck cancer and thyroid cancer.

Preferably, the inflammatory disease is selected from the group consisting of Multiple sclerosis, rheumatoid arthritis.

### EXAMPLES OF THE INVENTION

A CD73-PET ligand was synthesized as described below. The unlabeled as well as the ¹⁸F-labeled CD73 antagonist has been obtained. Compound 2 represents the first PET ligand developed for the labeling and imaging of CD73.

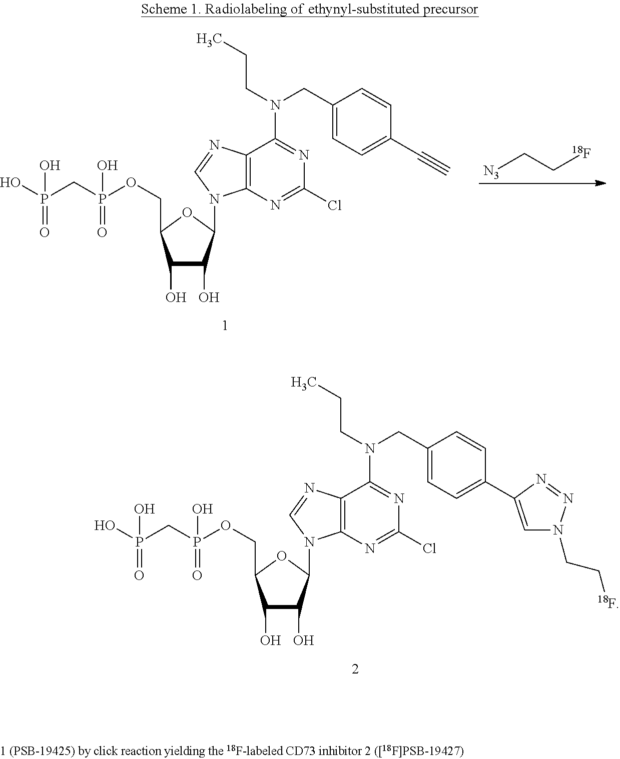

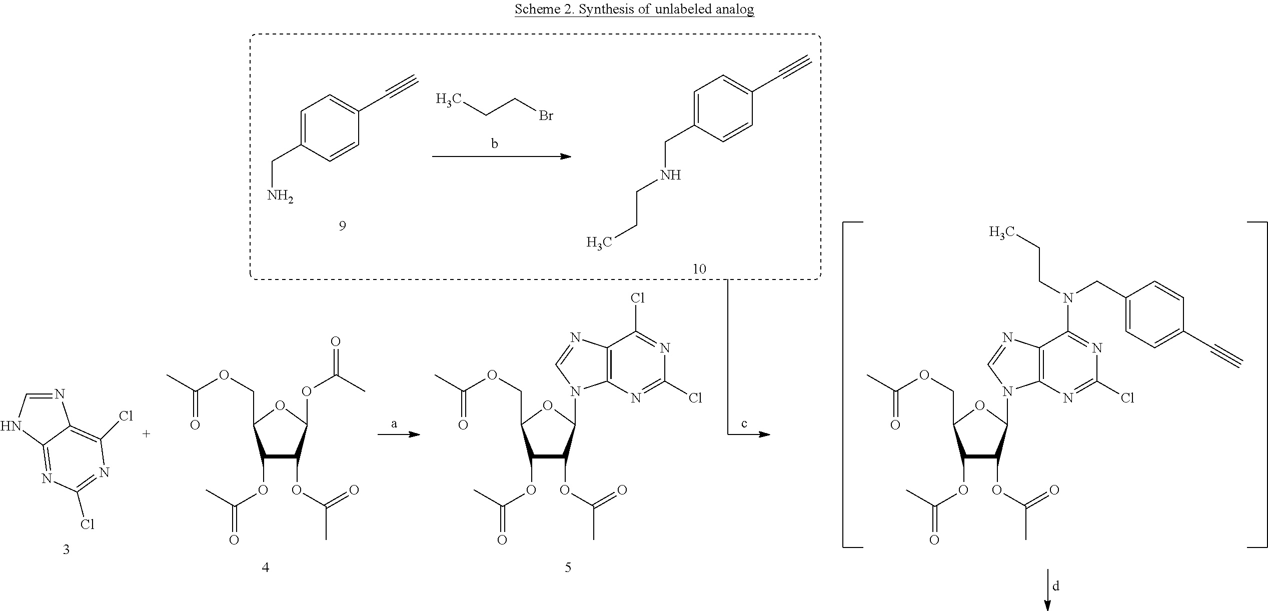

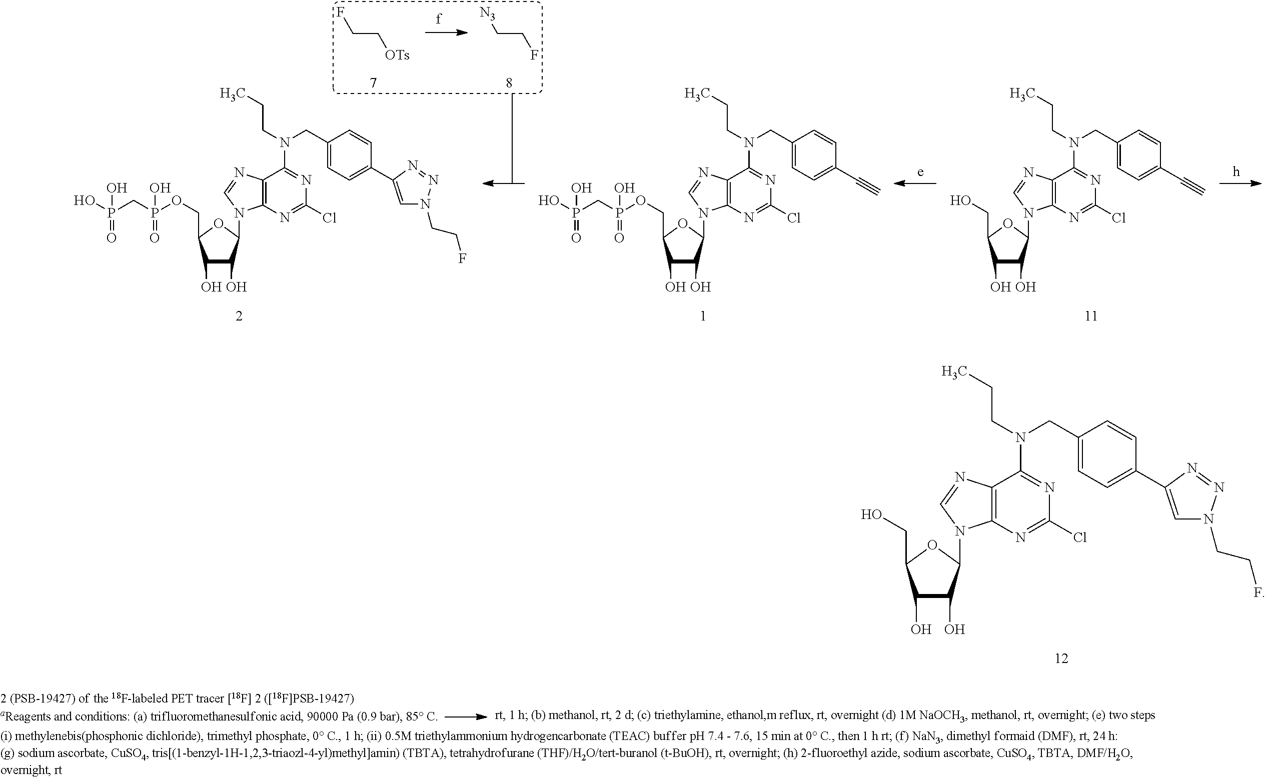

### Synthesis of Unlabeled Compound

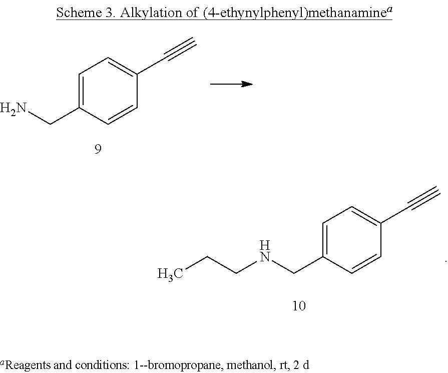

Compound 10 (N-(4-ethynylbenzyl)propan-1-amine) was synthesized by alkylation of compound 9 with 1-bromopropane in methanol.

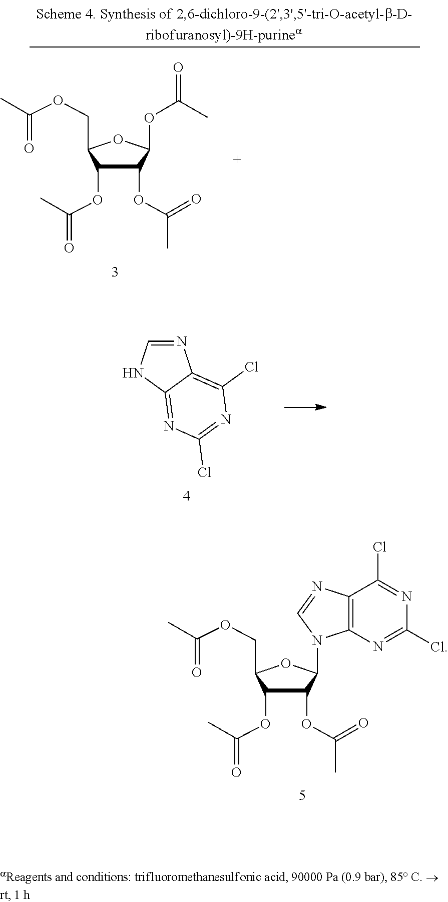

2,6-Dichloro-9-(2′,3′,5′-tri-O-acetyl-β-D-ribofuranosyl)-9H-purine (5) was synthesized starting from 1,2,3,5-tetraacetyl-β-D-ribofuranose (3) and 2,6-dichloropurine (4). The reaction was carried out using previously published reaction conditions.¹³ Both compounds were melted at 85° C. and reacted after the addition of trifluoromethanesulfonic acid.

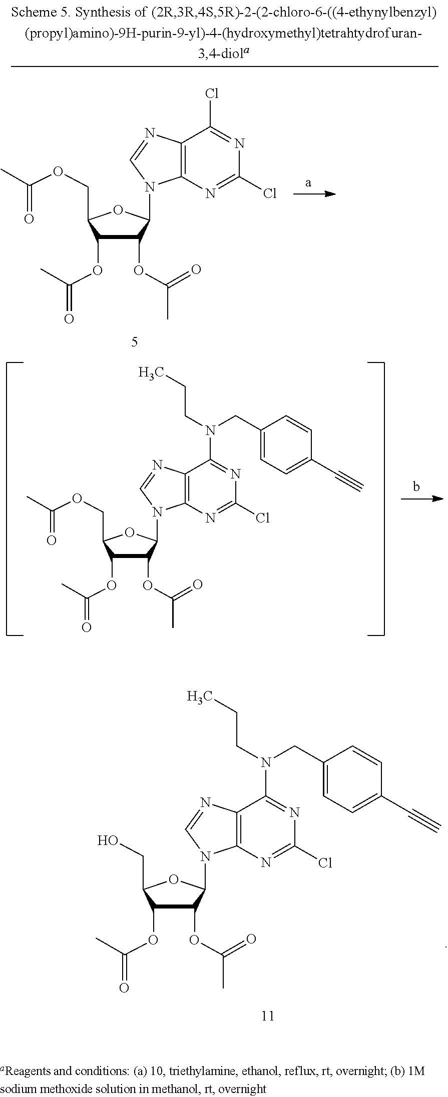

2′,3′,5′-Tri-O-acetyl-2,6-dichlororibofuranosylpurine (5) was reacted with N-(4-ethynylbenzyl)propan-1-amine (10) under basic conditions in the presence of triethylamine in ethanol. The reaction was carried out under reflux overnight, which yielded, besides the desired product, also partially deacetylated derivatives of 6. The product was not isolated but directly employed for the next step to afford the fully deacetylated product 11 by treatment with 1 M sodium methoxide solution in methanol.

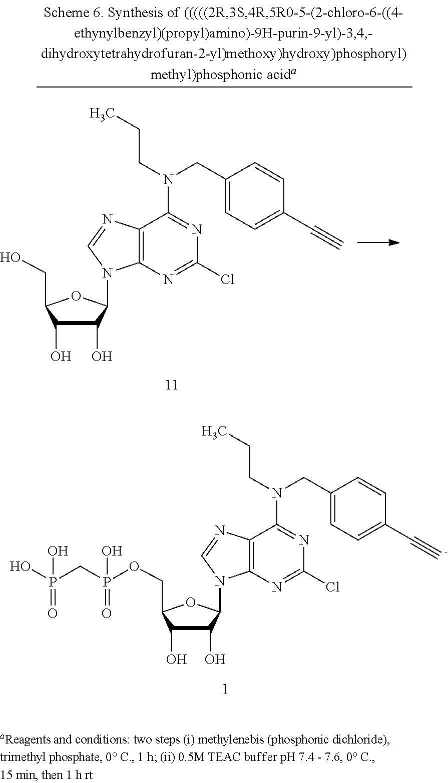

The phosphonylation reaction and subsequent hydrolysis were performed according to previously reported methods with some modifications.^{13, 14, 15 16} Phosphonylation was carried out by reaction of 11 with methylenebis(phosphonic dichloride) in trimethyl phosphate upon ice-cooling for 1 h. Final hydrolysis was achieved using freshly prepared 0.5 M TEAC buffer. Extraction with tert-butylmethyl ether (TBME) and subsequent purification by reversed-phase high-performance liquid chromatography (RP-HPLC) afforded the desired product 1.

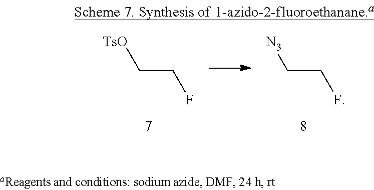 [SMILES: Cc1ccc(S(=O)(=O)OCCF)cc1 ; [N-]=[N+]=NCCF]

2-Fluoroethyl-4-toluenesulfonate (7) was dissolved in anhydrous DMF. After addition of sodium azide, the reaction mixture was stirred at rt for 24 h and monitored by TLC. Since the isolation of 1-azido-2-fluoroethane (8) might lead to an explosion, the crude mixture was filtered and used without further purification for the following reaction step.

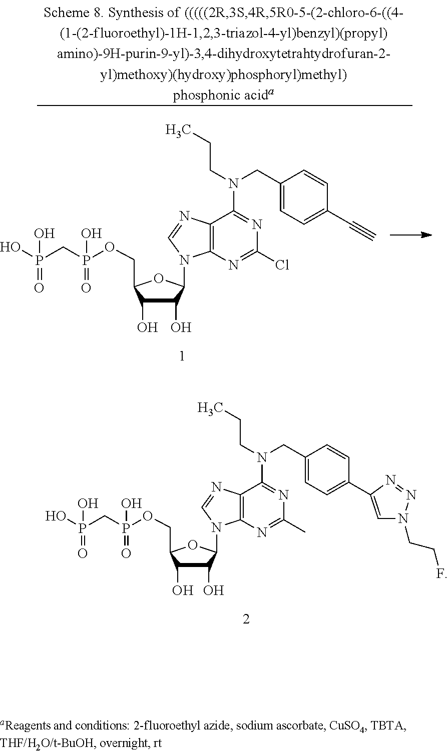

In order to obtain the desired final product 2, (((((2R,3S,4R,5R)-5-(2-chloro-6-((4-ethynylbenzyl)(propyl)amino)-9H-purin-9-yl)-3,4-dihydroxytetrahydrofuran-2-yl)methoxy)(hydroxy)-phosphoryl)methyl)phosphonic acid (1) was coupled to 1-azido-2-fluoroethane (8) via azide-alkyne Huisgen cycloaddition using TBTA, copper sulfate and sodium ascorbate in a mixture of THF, H₂O and t-BuOH. Purification by RP-HPLC afforded the desired product 2 in a yield of 18%.

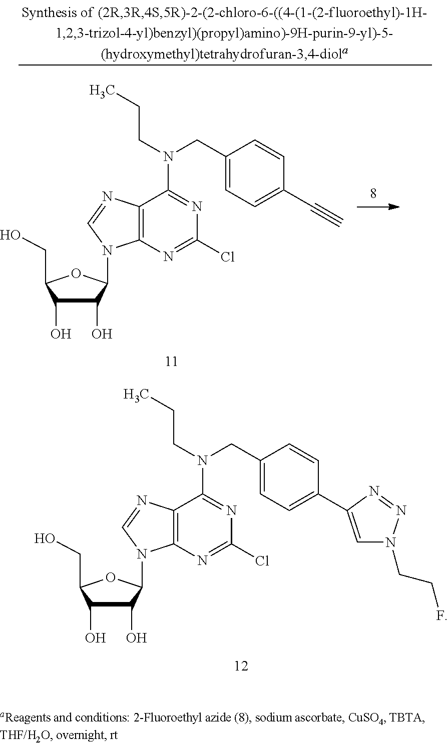

(2R,3R,4S,5R)-2-(2-Chloro-6-((4-(1-(2-fluoroethyl)-1H-1,2,3-triazol-4-yl)benzyl)(propyl)-amino)-9H-purin-9-yl)-5-(hydroxymethyl)tetrahydrofuran-3,4-diol (12) was synthesized by employing azide-alkyne Huisgen cycloaddition reaction conditions. To crude 1-azido-2-fluoroethane (8), dissolved in DMF, 11 and TBTA were added. Sodium ascorbate and copper sulfate were dissolved in water and then added to the reaction mixture. The mixture was stirred at ambient temperature overnight. Extraction and subsequent chromatographic purification on silica gel yielded the desired product 12 (73%).

### [¹⁸F]-[((2R,3S,4R,5R)-5-{2-chloro-6-[(4-{1-[2-(fluoro)ethyl]-1H-1,2,3-triazol-4-yl}benzyl)(propyl)amino]-9H-purin-9-yl}-3,4-dihydroxytetrahydrofuran-2-yl)methoxy]methylenebisphosphonic acid ([¹⁸F]2)

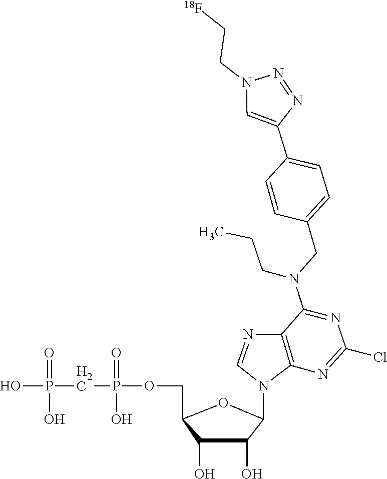

In a computer controlled TRACERLab Fx_{FDG} Synthesizer a batch of aqueous [¹⁸F]F-ions (3.1-5.2 GBq) from the cyclotron target was passed through an anion exchange resin (pre-conditioned Sep-Pak® Light QMA cartridge with carbonate counter-ion). [¹⁸F]F-ions were eluted from the resin with a mixture of 1 M K₂CO₃ (aqueous, 40 μL), water for injection (WFI, 200 μL), and acetonitrile (800 μL, DNA-grade) containing Kryptofix® 2.2.2 (K_{2.2.2}, 20 mg, 53 μmol) in the reactor. Subsequently, the aqueous K(K_{2.2.2})[¹⁸F]F solution was carefully evaporated to dryness in vacuo. An amount of precursor compound 2-azidoethyl 4-methylbenzenesulfonate (20 mg, 83 μmol) in acetonitrile (DNA-grade, 500 μL) was added and the mixture was heated at 110° C. for 3 min. Meanwhile, the labeled 1-azido-2-[¹⁸F]fluoroethane was distilled from the reactor into an ice-cooled 10 mL flask that contained a mixture of 1 (5.0 mg, 8.1 μmol) in DMF (300 μL), CuSO₄·5H₂O (40 mg, 160 μmol) in HEPES buffer (pH: 5.7, 100 μL) and sodium ascorbate (63 mg, 318 μmol) in HEPES buffer (pH: 5.7, 100 μL). After 30 min stirring at 60° C., the mixture was passed through a PTFE sterile filter (0.2 μm). The filter was rinsed with DMF (500 μL) and then with WFI (500 μL). The combined filtrate and rinsing solutions were purified by gradient-radio-HPLC system A (method A). The product fraction of compound [¹⁸F]2 (retention time t_{R}([¹⁸F]2)=12.2 min) was collected in a flask pre-treated with Sigmacote® and solution was evaporated to dryness in vacuo. The residue was redissolved in WFI/EtOH (1 mL, 9:1 v/v). Product compound [¹⁸F]2 was obtained in an overall radiochemical yield of 21.2±3.0% (decay-corrected, based on cyclotron-derived [¹⁸F]F-ions, n=21) in 119±10 min from the end of radionuclide production. [¹⁸F]2 was isolated in radiochemical purities of >99% with molar activities in the range of 2.3-54.6 GBq/μmol at the end of the synthesis. Radiochemical purities and molar activities of [¹⁸F]2 (retention time t_{R}([¹⁸F]2)=9.4 min) were determined by analytical radio-HPLC B (method B).

### In Vitro Stability in Mouse and Human Serum.

The serum stability of the radioligand [¹⁸F]2 was evaluated by incubation in mouse serum at 37° C. for up to 90 min. An aliquot of formulated [¹⁸F]2 (20 μL, 3.9 MBq) was added to a sample of mouse serum (200 μL), and the mixture was incubated at 37° C. Samples of 20 μL each were drawn after periods of 10, 30, 60 and 90 min and quenched in ice-cold acetonitrile (100 μL, DNA-grade) followed by centrifugation (3000 rpm) for ≥5 min. The supernatant was analyzed by analytical radio-HPLC B (method B). Serum stability investigations in human serum were performed analogously.

Determination of the log D_{7.4}-value of [¹⁸F]2. The lipophilicity of [¹⁸F]2 was determined following the procedure described by Prante et al.¹⁷ Triazole [¹⁸F]2 (˜400 kBq) in PBS buffer (10 μL, pH 7.4) was added to PBS buffer (590 μL, pH 7.4) and octan-1-ol (600 μL). The two-layer mixture was shaken for 3 min on a vortex mixer at RT and centrifuged (3000 rpm) for 5 min. The main part of the octanol layer (400 μL) was collected carefully and added to a new tube with PBS buffer (400 μL, pH 7.4). The two-layer mixture was shaken again for 10 min and centrifuged for 5 min (3000 rpm). Three samples were prepared and two aliquots (á 100 μL) of both layers were measured in a g-counter 2480 Wizard2 (Perkin-Elmer, Waltham, USA). The partition coefficient was determined by dividing cpm(octanol) by cpm(PBS) and indicated as log D_{7.4} (exp.).

Partition coefficient (log D (exp.)). [¹⁸F]2 is a hydrophilic compound with a log D (exp.) of −0.12±0.03. In comparison to the calculated log P values (clog P) of 0.22² and 0.47³ the measured solubility of [¹⁸F]2 in PBS-buffer in the biphasic 1-octanol/PBS system is increased by factor 2.2 and 3.9, respectively.
² calculated log P value (clog P) was calculated by ChemBioDraw Ultra 13.0
³ calculated log P value (clog P) was calculated by ACD/Chemsketch freeware

Determination of plasma protein binding (PPB) was done following the procedure of Börgel et al.²⁴

For the quantification of different analytes, a LC system was coupled with a single quadrupole (SQ) mass spectrometer.

UPLC-UV/MS (Agilent, Waldbronn, Germany): pump: 1260 Bin Pump (G1212B); degasser: 1260 HiP (G4225A); column oven: 1290 TCC (G1316C), 30° C.; autosampler 1260 HiP ALS (G1367E), 1 □L injected if not stated otherwise; UV/vis detector 1260 VWD (G1314F); MS source: multimode source (G1978B); MS-Detector 6120 Quadrupole (G1978B); MS parameters: Vaporizer temperature: 250° C.; drying gas: 10 L/min; nebulizer pressure: 40 psi; capillary voltage: 3000 V; fragmentor voltage: 100 V; drying gas temperature: 350° C.; LC parameters: precolumn: Chiralpak® HSA HPLC Guard Column (2.0×10 mm, 5 μm particle size); main column: Chiralpak® HSA HPLC Column (2.0×50 mm, 5 μm particle size, Daicel, Eschborn, Germany); temperature: 25° C.; mobile phase: aqueous ammonium acetate solution (50 mM, pH 7.4)/i-propanol 96:4; flow rate: 0.3 mL/min; isocratic.

Reference compounds and 2 were dissolved in methanol and retention times were measured in LC-MS. D-glucose was used to determine the dead time (0.75 min) of the system. Reference compounds were used to plot k′/(k′+1) against known PPB values (Table 1, 2).

$${k}^{′} = \frac{{t}_{R} - {t}_{D}}{{t}_{D}}$$

With k′=retention factor
   t_{R}=retention time [min]
   t_{D}=death time [min]

TABLE 1
Measured retention times and known
PPB values of reference compounds.

| Compound | t_{R} (n = 3) | k′ | k′/(k′ + 1) | PPB [%]^{25, 26} |
|---|---|---|---|---|
| Metronidazol | 0.87 | 0.16 | 0.14 | 11.0 |
| Paracetamol | 0.99 | 0.33 | 0.25 | 24.0 |
| Salbutamol | 1.10 | 0.47 | 0.32 | 7.5 |
| Sulfamethoxazol | 1.32 | 0.77 | 0.43 | 66.0 |
| Ramipril | 1.54 | 1.06 | 0.51 | 56.0 |
| Phenytoin | 3.83 | 4.12 | 0.80 | 91.0 |
| Propranolol | 4.05 | 4.42 | 0.82 | 87.0 |
| Imipramine | 8.35 | 10.17 | 0.91 | 92.6 |
| Haloperidol | 8.83 | 10.80 | 0.92 | 92.0 |
| Chlorpromazine | 33.51 | 43.82 | 0.98 | 97.8 |

Triple-negative breast cancer (TNBC) is often associated with CD73-upregulation. This makes CD73 not only a good target for cancer immunotherapy but also offers the opportunity for targeting CD73 for diagnosis. The use of highly potent nucleotide-derived CD73 inhibitors as diagnostic tools bearing fluorescent, ultrasonic labels or radionucleotides might ease the early localization of primary tumors and metastasis in TNBC, improving the prognosis.

### Design of Fluorescence-Labeled CD73 Inhibitor

Schmies et al. recently reported on the synthesis of a fluorescein-labeled AOPCP-derived CD73 inhibitor. The p-position of the N⁶-benzyl-substituent was found to be suitable for the introduction of a linker, which carries the fluorescein moiety.¹⁸ However, initial plans to synthesize a 2-chloro substituted analog had failed.¹⁸ Since additional substitution of the 2-position by e.g. a chloro substituent might lead to improved properties,¹⁵ a new strategy to synthesize the desired fluorescence-labeled 2-chloro-substituted AOPCP derivative was developed.

The successful synthesis and pharmacological evaluation of the resulting (((((2R,3S,4R,5R)-5-(2-chloro-6-((4-((6-(3′,6′-dihydroxy-3-oxo-3H-spiro[isobenzofuran-1,9′-xanthene]-6-carboxamido)-hexyl)carbamoyl)benzyl)amino)-9H-purin-9-yl)-3,4-dihydroxytetrahydrofuran-2-yl)methoxy)(hydroxy)phosphoryl)methyl)phosphonic acid is described below.

### Chemistry—Synthesis of Fluorescence-Labeled CD73 Inhibitor

6-Carboxyfluorescein (13) was attached to commercially available N-boc-1,6-hexanediamine via amide coupling using O-(7-azabenzotriazol-1-yl)-N,N,N′,N′-tetramethyluronium-hexafluorphosphat (HATU) as a coupling reagent, and N,N-diisopropylethylamine (DIPEA) as a base in DMF to give 14.¹⁹ Subsequent boo-deprotection using trifluoroacetic acid (TFA) in dichloromethane (DCM) yielded 15.¹⁸ Next, 4-(boo-aminobenzyl)benzoic acid was coupled to the primary amino group of 15 again employing HATU and DIPEA to give 16, followed by boc-deprotection with TFA in DCM yielding 17.^{18, 19} In a parallel reaction, commercially available 2,6-dichloro-9-(β-D-ribofuranosyl)purine (18) was phosphonylated using methylenbis(phosphonic dichloride) in trimethyl phosphate, followed by quenching with aqueous triethylammonium hydrogencarbonate (TEAC) buffer according to a previously published, optimized method.^{13, 18} RP-HPLC purification afforded the pure product 19. In the final reaction step, the nucleophilic substitution of 19 by the primary amine 17 in absolute ethanol in the presence of triethylamine under reflux conditions, followed by RP-HPLC purification, yielded the desired final product 20 (yield 23%).¹⁸ The structure of 20 was confirmed by ¹H, ¹³C and ³¹P-NMR spectroscopy and LC/ESI-(UV)MS analysis (positive and negative mode) indicating a purity of >95%.

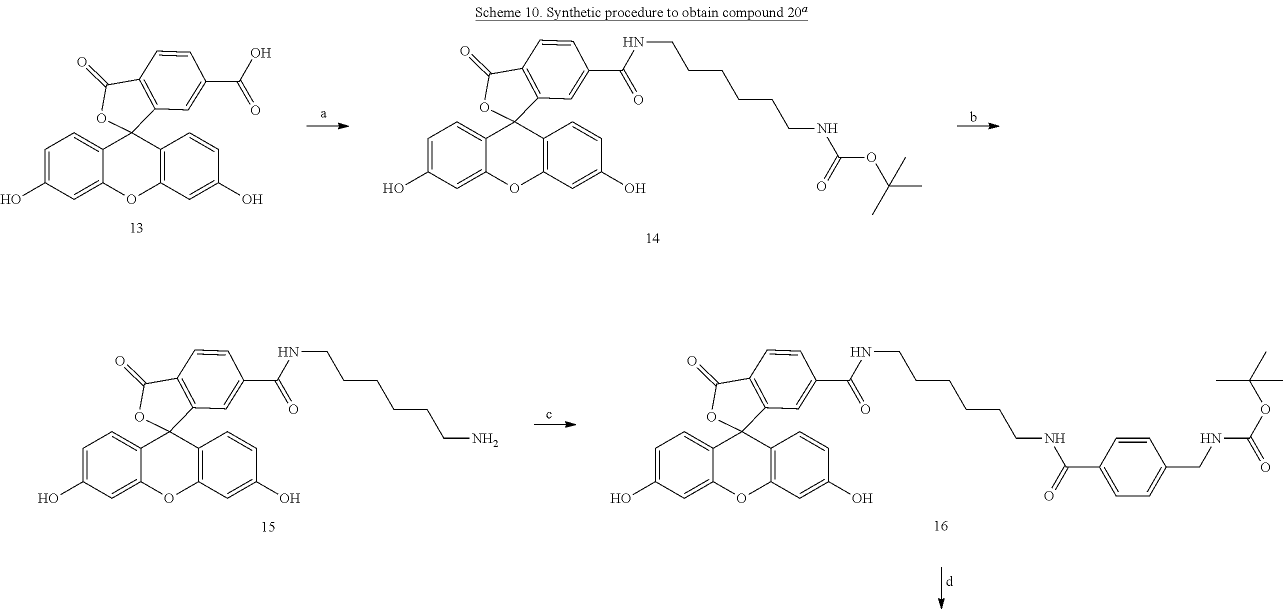

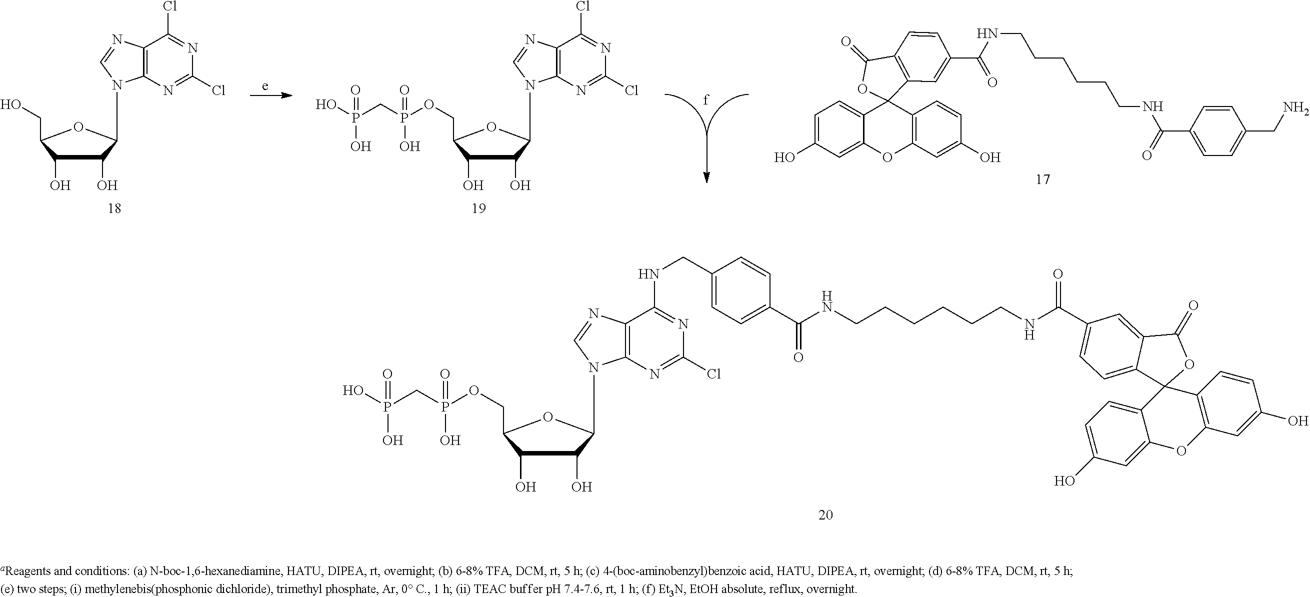

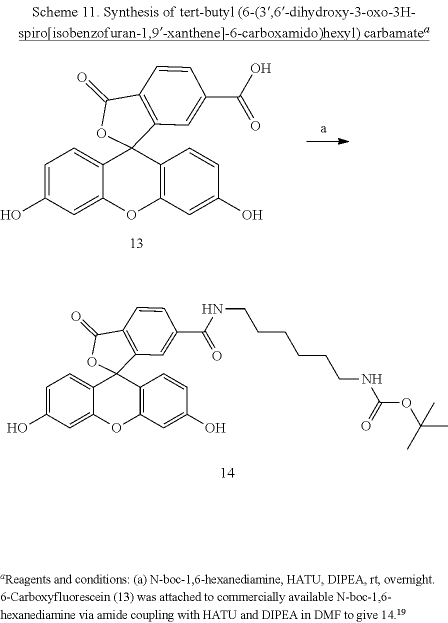 [SMILES: O=C(O)c1ccc2c(c1)C1(OC2=O)c2ccc(O)cc2Oc2cc(O)ccc21 ; CC(C)(C)OC(=O)NCCCCCCNC(=O)c1ccc2c(c1)C1(OC2=O)c2ccc(O)cc2Oc2cc(O)ccc21]

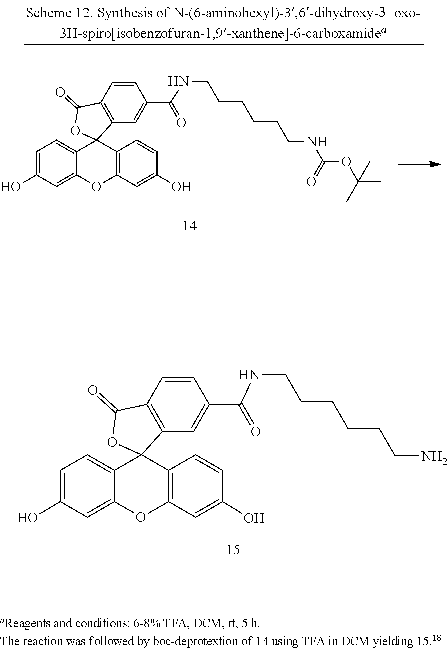 [SMILES: CC(C)(C)OC(=O)NCCCCCCNC(=O)c1ccc2c(c1)C1(OC2=O)c2ccc(O)cc2Oc2cc(O)ccc21 ; NCCCCCCNC(=O)c1ccc2c(c1)C1(OC2=O)c2ccc(O)cc2Oc2cc(O)ccc21]

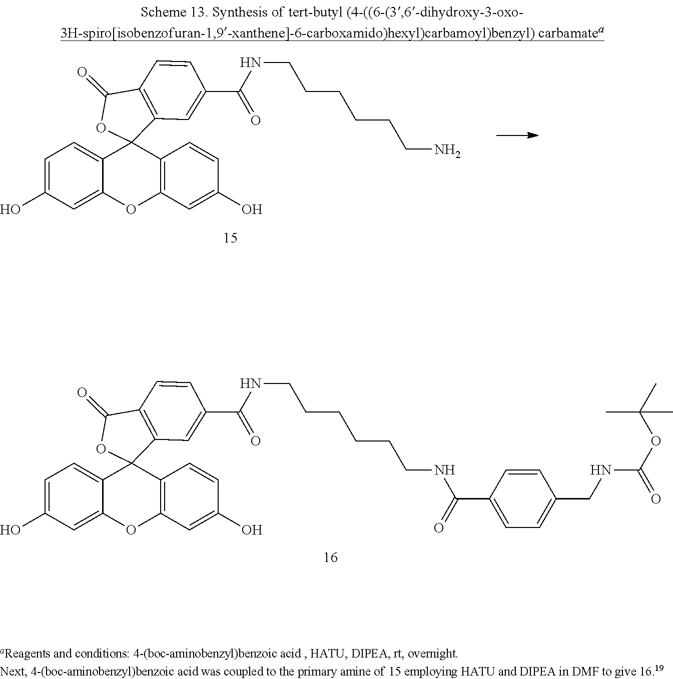 [SMILES: NCCCCCCNC(=O)c1ccc2c(c1)C1(OC2=O)c2ccc(O)cc2Oc2cc(O)ccc21 ; CC(C)(C)OC(=O)NCc1ccc(C(=O)NCCCCCCNC(=O)c2ccc3c(c2)C2(OC3=O)c3ccc(O)cc3Oc3cc(O)ccc32)cc1]

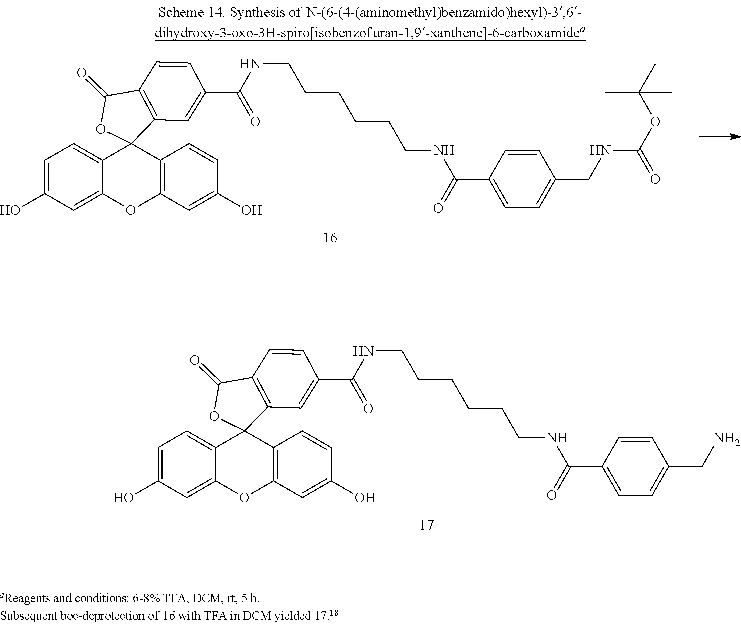 [SMILES: CC(C)(C)OC(=O)NCc1ccc(C(=O)NCCCCCCNC(=O)c2ccc3c(c2)C2(OC3=O)c3ccc(O)cc3Oc3cc(O)ccc32)cc1 ; NCc1ccc(C(=O)NCCCCCCNC(=O)c2ccc3c(c2)C2(OC3=O)c3ccc(O)cc3Oc3cc(O)ccc32)cc1]

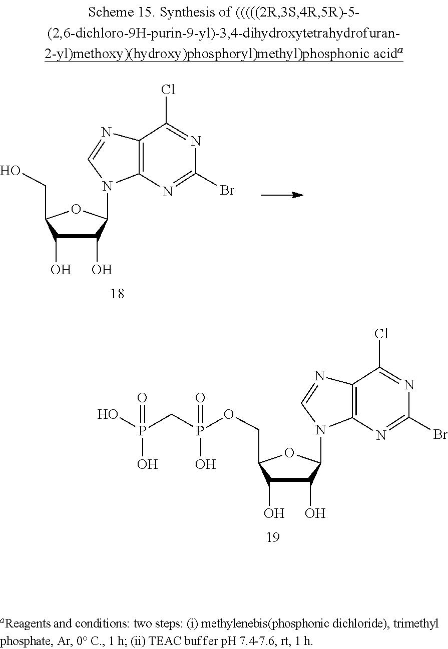

Commercially availabe 2,6-dichloro-9-(β-D-ribofuranosyl)purine (18) was phosphonylated using methylenbis(phosphonic dichloride) in trimethyl phosphate, followed by quenching with aqueous TEAC buffer. Both reaction steps were carried out according to an optimized previously published method affording 19.^{13, 18}

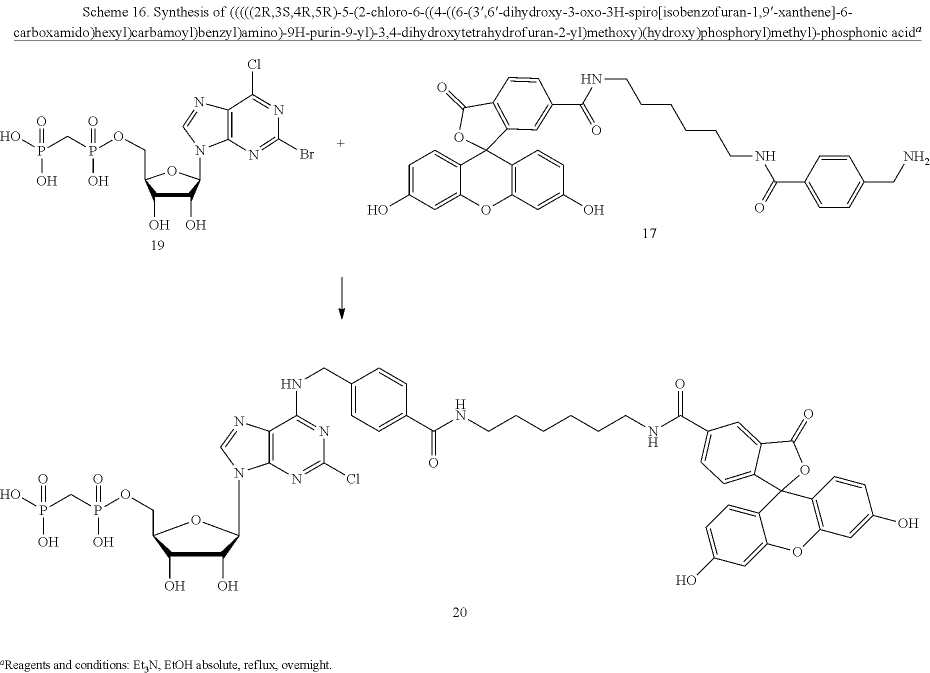

In the final reaction step, nucleophilic substitution of 19 by the primary amine 17 in absolute ethanol in the presence of triethyl amine under reflux conditions, followed by RP-HPLC purification, yielded the desired final product 20 (yield 23%).¹⁸

### Pharmacological Evaluation

Compound 2b, its precursor 1, and 20 were investigated for their CD73-inhibitory potency using tritium-labeled AMP as a substrate.²⁰ After incubation with CD73, non-hydrolyzed substrate and inorganic phosphate were precipitated using a solution of lanthanum chloride, and the precipitate was separated from the solution containing the reaction product [2,8-³H]adenosine by filtration through GF/B glass fiber filters (WhatmanTM GE Healthcare, Chicago, IL, USA) using a cell harvester (M-48, Brandel, Gaitherburg, MD, USA). The filtrates (ca. 1.8 mL each) were transferred to scintillation vials, scintillation cocktail ULTIMA Gold XR (PerkinElmer, Waltham, MA, USA) was added (5 mL), and the radioactivity esd quantified by scintillation counting. CD73 preparations were (1) recombinant soluble human CD73,¹³ (2) recombinant soluble rat CD73,²¹ both expressed in Spodoptera frugiperda 9 (Sf) cells, (3) membrane preparations of MDA-MB-231 cells,¹⁶ a human triple-negative breast cancer cell line with high expression of CD73,¹⁸ and (4) membrane preparations of the 4T1.2 cell line, a mouse CD73-expressing breast cancer cell line.²² The potency of the compounds was determined by measurements of full concentration-inhibition curves. K values were calcuated from the obtained IC₅₀ values employing the Cheng-Prusoff equation.²³

TABLE 1
Potencies of compounds 1, 2 and 20
at different CD73 preparations.^{a}

| | | Human | |
|---|---|---|---|
| | Human soluble | CD73MDA-MB- | Rat soluble |
| | CD73 | 231 | CD73 |
| Compd. | Kᵢ ± SEM^{a} (nM) | Kᵢ ± SEM^{a} (nM) | Kᵢ ± SEM^{a, b} (nM) |
| 1 | 0.372 ± 0.030 | n.d. | n.d. |
| 2 | 1.02 ± 0.11 | 2.78 ± 0.47 | 6.09 ± 0.74 |
| 20 | 5.96 ± 0.850 | 6.76 ± 1.25 | 18.0 ± 1.43 |

^{a}[2,8-³H]AMP (5 μM) was used as substrate; Kₘ value 59 μM, 17 μM and 14.8 μM respectively for purified recombinant soluble human CD73, native membrane-anchored human CD73 (in MDA-MB-231 cell membrane preparations), and purified recombinant soluble rat CD73;
n.d.—not determined.

### Inhibition of Human NTPDases

Inhibitory activity of the CD73 inhibitors 2 and 20 at human NTPDases 1, 2, 3, and 8 was investigated using ATP (100 μM) as a substrate according to previously published procedures.²⁴ ATP and 2 were co-incubated with recombinantly expressed human NTPDases at 37° C. for 30 min, and the enzymatic reaction was terminated by heating at 90° C. for 10 min. The amount of enzyme preparation was adjusted to ensure 10-20% of substrate conversion. The formed products were separated by capillary electrophoresis (CE) and individually quantitated by diode array detection (DAD) at a wavelength of 260 nm. For inhibition analysis, at least three independent experiments were performed each in triplicate. The inhibition of the enzyme activity was calculated in relation to the positive control without inhibitor and plotted by GraphPad Prism 8 software (GraphPad software, San Diego, CA, USA) (for results see Table 2 and Table 3).

### Effect on ADP Receptors

Interaction of 2 with the ADP-activated human P2Y₁ receptor, which is Gq protein-coupled, was determined by calcium mobilization assays. The receptor was stably expressed in 1231N1 astrocytomna cells^{25, 26} (for results see Table 2). Compound 20 was not tested at the P2Y₁ receptor since the fluorescent compound would interfere with the calcium mobilization assay.

The interaction of 2 with the human P2Y₁₂ receptor was investigated in a β-arrestin recruitment assay using the the galactosidase complementation technology as previously described.²⁵⁻²⁷ The human P2Y₁₂ receptor was expressed in CHO-PK1 cells (Eurofins DiscoverX, Fremont, CA, USA). For results see Table 2 and Table 3.

TABLE 2
Interaction of compound 2 with human NTPDases and the human
ADP-activated G protein-coupled receptors P2Y₁ and P2Y₁₂

| | IC₅₀ or EC₅₀ (nM) |
|---|---|
| | (% inhibition ± SEM at the indicated |
| | concentration^{a} or % activation ± |
| Target | SEM) |
| NTPDase1 | IC₅₀ > 50,000 |
| NTPDase2 | IC₅₀ > 50,000 |
| NTPDase3 | IC₅₀ > 50,000 |
| NTPDase8 | IC₅₀ > 50,000 |
| Inhibition P2Y₁ receptor | IC₅₀ > 10,000 |
| Activation P2Y₁ receptor | EC₅₀ > 10,000 |
| Inhibition P2Y₁₂ receptor | IC₅₀ > 10,000 |
| Activation P2Y₁₂ receptor | EC₅₀ > 10,000 |

^{a}For antagonist testing, 2-methylthio-ADP was employed as agonist at its EC₈₀ concentration (3 μM).

TABLE 3
Interaction of compound 20 with human NTPDases and the
human ADP-activated G protein-coupled receptors P2Y₁₂

| | IC₅₀ or EC₅₀ (nM) |
|---|---|
| | (% inhibition ± SEM at the indicated |
| | concentration^{a} or % activation ± |
| Target | SEM) |
| NTPDase1 | IC₅₀ = 27,600 ± 5300 |
| NTPDase2 | IC₅₀ < 50,000 |
| NTPDase3 | IC₅₀ = 27,700 ± 2700 |
| NTPDase8 | IC₅₀ < 50,000 |
| Inhibition P2Y₁₂ receptor | IC₅₀ > 10,000 |
| Activation P2Y₁₂ receptor | EC₅₀ > 10,000 |

^{a}For antagonist testing, 2-methylthio-ADP was employed as agonist at its EC₈₀ concentration (3 μM).

### Experimental Details for Compound Synthesis

### General

All reagents were commercially obtained from various producers (Acros, Aldrich, Fluka, Merck, Sigma etc.) and used without further purification. Commercial solvents of specific reagent grades were used without additional purification or drying. The reactions were monitored by thin-layer chromatography (TLC) using aluminum sheets with silica gel 60 F₂₅₄ (Merck) and dichloromethane/methanol mixtures (99:1 to 3:1) as eluents. Column chromatography was carried out on silica gel 0.060-0.200 mm, pore diameter ca. 6 nm. Mass spectra were recorded on an API2000 mass spectrometer (ABSciex) with an ESI-source coupled to an HPLC HP1100 apparatus (Agilent) with an EC50/2 Nucleodur C18 Gravity 3 μm column (Macherey-Nagel). The samples were dissolved in a mixture of H₂O/methanol containing 2 mM ammonium acetate. A sample of 8 μl was injected and a flow rate of 0.3 ml/min was applied. Elution was performed with a gradient of water/methanol from 90:10 to 0:100 in 10 min. The column was subsequently flushed for 10 min with 100% methanol containing 2 mM ammonium acetate. Positive total ion scans were observed from 150-800 m/z. The UV absorption was detected from 190-900 nm using a diode array detector (DAD). Purity was determined at 220-400 nm. Purities of all tested compounds were 295%. High resolution spectra were recorded on a micrOTOF-Q mass spectrometer (Bruker) with an ESI-source coupled to a HPLC Dionex Ultimate 3000 (Thermo Scientific) apparatus using flow injection mode. The sample solution was injected and a flow rat of 0.3 ml/min was applied. Elution was performed with acetonitrile containing 0.1% acetic acid, or 0.1% formic acid, respectively. Positive or negative full scan MS was observed from 50-1000 m/z. Sodium acetate or sodium formate was used as internal calibrant. ¹H, ¹³C and ³¹P-NMR spectra were performed on a Bruker AVANCE 500 or a Bruker AVANCE III HD 600 MHz spectrometer. DMSO-d₆, MeOD-d₄, or D₂O was used as solvent. ³¹P NMR spectra were recorded at room temperature; orthophosphoric acid (85%) was used as an external standard. Shifts are given in ppm relative to the external standard (³¹P NMR spectra) or relative to the remaining protons of the deuterated solvents used as internal standard (¹H, ¹³C NMR). Melting points were determined on a Büchi 530 melting point apparatus and are uncorrected. Preparative HPLC was carried out on a Knauer Smartline 1050 HPLC system equipped with a Knauer Eurospher-100 C18 column, 250 mm×20 mm, particle size 10 μm. UV absorption was detected at 254 nm. For lyophilization, a freeze dryer (CHRIST ALPHA 1-4 LSC) was used.

Radiochemistry. General Methods.

The first step of the radiosynthesis was carried out on a modified PET tracer radiosynthesizer (TRACERLab Fx_{FDG}, GE Healthcare). The recorded data was processed by the TRACERLab Fx software (GE Healthcare). Separation and purification of the radiolabeled compounds were performed on the semipreparative radio-HPLC system A: K-500 and K-501 pump, K-2000 UV detector (Herbert Knauer GmbH), NaI(TI) Scintibloc 51 SP51 γ-detector (Crismatec) and an ACE 5 AQ column (250 mm×10 mm). Method A started with a linear gradient from 10% to 90% CH₃CN in water (0.1% TFA) over 30 min, holding for 5 min and followed by a linear gradient from 90% to 10% CH₃CN in water (0.1% TFA) over 5 min, with I=254 nm and a flow rate of 5.0 mL min⁻¹. Radiochemical purities and molar activities were determined using the analytical radio-HPLC system B: Two Smartline 1000 pumps and a Smartline UV detector 2500 (Herbert Knauer GmbH), a GabiStar γ-detector (Raytest Isotopenmessgeräte GmbH) and a Nucleosil 100-5 C-18 column (250 mm×4 mm). Method B started with a linear gradient from 10% to 100% CH₃CN in water (0.1% TFA) over 15 min, holding for 3 min followed by a linear gradient from 100% to 10% CH₃CN in water (0.1% TFA) over 2 min, with I=254 nm and a flow rate of 1.0 mL min⁻¹. The recorded data of both HPLC-systems were processed by the GINA Star software (Raytest Isotopenmessgeräte GmbH). No-carrier-added aqueous [¹⁸F]fluoride was produced on a RDS 111e cyclotron (CTI-Siemens) by irradiation of a water target (2.8 mL) using 10 MeV proton beams on 97.0% enriched [¹⁸O]H₂O by the ¹⁸O(p,n)¹⁸F nuclear reaction.

### Recombinant Soluble CD73

Human soluble CD73 was expressed in Spodoptera frugiperda (Sf9) insect cells and purified as previously described.¹⁶ The cDNA encoding for the mature human CD73 (residues 27-549) fused to 6×His-tag at the C-terminus (Genbank accession no. NM_002526) corresponding to the natural variant T376A (P21589/VAR_022091, UniProtKB/Swiss-Prot) was ligated into the pAcGP67B vector. For transfection, 1 μl of the recombinant vector (1000 ng/μl) mixed with 2.5 μl of baculovirus genomic DNA ProEasy™ (AB vector, CA, USA) was used to transfect Sf9 cells grown in Insect-XPRESS™ medium (#BE12-7300, Lonza, Switzerland) supplemented with 10 mg/l gentamicin. The produced soluble enzyme was then concentrated by ultrafiltration with Amicon® Ulta-15 filters, 10 KDa cut-off (Merck Milipore, MA, USA), and then subjected to metal affinity chromatography (IMAC) purification with HisPur™ Ni-NTA spin columns (#88226, Thermo Fisher Scientific, MA, USA) according to the manufacturer's protocol. The purified enzyme was aliquoted and stored at −80° C. until further use. A previously published method was used to express the glutathione-S-transferase fusion protein of soluble rat CD73 in Sf9 insect cells.²¹

### Cell Culture

Human triple-negative breast cancer cells (MDA-MB-231), which natively express CD73, were grown in Dulbecco's Modified Eagle Medium (DMEM, #: 41966, Thermo Fisher Scientific, MA, USA) supplemented with 10% fetal bovine serum (FBS, #: P30-1502, PAN Biotech, Germany) and 100 U/ml penicillin+100 μg/ml streptomycin (#: P06-07100, PAN Biotech, Germany). Cells were incubated at 37° C. with 5% CO₂ for 72 h to reach confluence (80-90%). Confluent cells were washed with phosphate-buffered saline (PBS), then detached by a 5-min incubation with trypsin/EDTA (0.05%/0.6 mM, #P10-022100, PAN Biotech, Germany). Detached cells were re-suspended in culture media and split 1:20.

### Membrane Preparations

Confluent cells grown in 175 cm² culture flasks were detached as described above. Culture dishes (150 cm²) were seeded with approx. 100 cells/dish and incubated at 37° C., 5% CO₂ for 4 days. The growth medium was discarded, and the dishes were washed with 10 ml PBS and frozen at −20° C. Frozen cells were scraped off the dishes with 1 ml of ice-cold buffer (50 mM Tris, 2 mM EDTA, pH 7.4), collected in a conical tube, then centrifuged at 1000 g at 4° C. for 10 min. Then, the pellets were resuspended in buffer (0.5 ml/dish; 25 mM Tris, 1 mM EDTA, 320 mM sucrose, pH 7.4, 1:1000 protease inhibitor cocktail #P8340, Sigma-Aldrich, MO, USA) and homogenized three times for 30 s each (20,500 rpm, Ultraturrax, IKA-Labortechnik, Germany). The homogenate was centrifuged for 10 min at 1000 g, 4° C., and the supernatant was collected and centrifuged for further 30 min at 48,000 g, 4° C. The obtained pellets were resuspended in washing buffer (0.5 ml/dish) and centrifuged using the same conditions. After three more washing steps, the pellets were resuspended in Tris buffer 50 mM, pH 7.4 (0.1 ml/dish), aliquoted, and stored at −80° C. until use.

### Enzyme Inhibition Assay

Compounds were tested using a previously described method.²⁰ The stock solutions were prepared in demineralized water, which were further diluted in reaction buffer (Tris 25 mM, NaCl 140 mM, sodium dihydrogen phosphate 25 mM, pH 7.4). For screening, 10 μl of each test compound were transferred into the respective test tube containing 70 μl of the reaction buffer. For determining concentration-response curves, 10 μl of different dilutions of test compounds were pipetted into the test tubes. A solution or suspension of soluble or membrane-bound CD73 (10 μl, soluble rat CD73: 1.63 ng; soluble human CD73: 0.365 ng; membrane preparation of MDA-MB-231 cells expressing CD73: 7.4 ng of protein per vial) was transferred into all test tubes except for the negative controls. The substrate [2,8-³H]AMP (specific activity 7.4×108 Bq/mmol (20 mCi/mmol)), American Radio-labeled Chemicals, MO, USA, distributed by Hartman Analytic, Germany) was added in a volume of 10 μl (5 μM final concentration) to initiate the reaction. After 25 min of incubation at 37° C. in a shaking water bath, the samples were cooled on ice, and 500 μl of precipitation buffer (lanthanum chloride, 100 mM in sodium acetate 100 mM, pH 4.0) was added to stop the reaction and enable precipitation. Samples were kept on ice for at least 30 min until complete precipitation was achieved, and then filtered through GF/B glass fiber filters using a Brandel cell harvester (M-48, Brandel, MD, USA). Reaction vials were washed three times with 400 μl of cold (4° C.) demineralized water each, then 5 ml of scintillation cocktail (ULTIMA Gold XR, PerkinElmer, MA, USA) was added, and radioactivity was measured using a scintillation counter (Tri-Carb 2900TR, Packard/PerkinElmer). All experiments were performed in duplicate, baseline-corrected and normalized against negative and positive controls, respectively. Three independent experiments were conducted, and data were analyzed using Prism-GraphPad 7 (GraphPad Software, La Jolla, USA). The Cheng-Prusoff equation was used to calculate the Ki values using the following Km values (Km, rat CD73: 53.0 μM; Km, human CD73: 17.0 μM; Km, (MDA-MB-231): 14.8 μM).²³

### Capillary Electrophoresis Assay for Human NTPDases 1,2,3 and 8

Test compounds were investigated at a concentration of 50 μM, and 100 μM ATP substrate according to published procedures.²⁴ Recombinantly expressed human NTPDases were employed. The amount of enzyme preparation was adjusted to ensure 10-20% of substrate conversion. The reaction buffer contained 10 mM HEPES, 2 mM CaCl₂, 1 mM MgCl₂, pH 7.4. The samples were incubated at 37° C. for 30 min, and the enzymatic reaction was terminated by heating at 90° C. for 10 min. After cooling the samples on ice, they were investigated by capillary electrophoresis (CE) with diode array detection (DAD) at a wavelength of 260 nm. For the inhibition analysis, at least three independent experiments were performed in triplicate (n=3). The inhibition of the enzyme activity was calculated in relation to the positive control without inhibitor and plotted in GraphPad Prism 8 software (GraphPad software, San Diego, CA, USA).

Analysis was carried out using P/ACE MDQ capillary electrophoresis system (Beckman Instruments, Fullerton, CA, USA). The separation was performed in a polyacrylamide-coated capillary [30 cm (10 cm effective length)×50 μm (id), ×360 μm (od)]. Before each run, the capillary was rinsed with the background electrolyte (50 mM phosphate buffer (pH 6.5)) for 1 min at 30 psi. Samples were electrokinetically injected by applying voltage of −6 kV for 30 s at the capillary outlet and the nucleotides were separated by voltage application of −15 kV and 0.2 psi pressure. Detection at a wavelength of 260 nm. Data collection and peak area analysis were performed by the P/ACE MDQ software 32 KARAT obtained from Beckman Coulter (Fullerton, CA, USA). Quantification by AMP and ADP external standard curves and corrected area under electropherogram peak at 260 nm.

### In Vivo Experiments

Animal models. All animal experiments were performed in accordance with the legal requirements of the European Community (Directive 2010/63/EU) and the corresponding German Animal Welfare Law (TierSchG, TierSchVersV) and were approved by the local authorizing agency (State Office for Nature, Environment and Consumer Protection North Rhine-Westphalia). MDA-MB-231 or AsPC-1 cells were cultivated in RPMI supplemented with 10% fetal calf serum and 100 U/mL penicillin and 100 μg/mL streptomycin. 5×10⁶ MDA-MB-231 or AsPC-1 cells were injected subcutaneously into the shoulder region of 10 to 12 week old NSG mice (NOD.Cg-Prkdcscidll2rgtm1Wjl/SzJ, Charles River Laboratories). Two tumors were inoculated per animal and growth was followed by digital caliper measurements (volume=½ (length*width²)).

In vivo Imaging. Adult C57bl/6 (biodistribution study, 21±1 g) or tumor bearing NSG mice (24±3 g) were anesthetized by isoflurane/O₂ and one lateral tail vein was cannulated using a 27 G needle connected to 15 cm polyethylene catheter tubing. ¹⁸F-PSB-19427 (˜450 kBq/g body weight) were injected as a bolus (50 μL compound flushed with 50 μL saline) via the tail vein and subsequent PET scanning was performed. In the blocking studies, a subgroup of tumor-bearing mice was injected with ˜1000 fold excess of unlabeled PSB-19427 10 minutes prior to radiotracer injection. PET imaging studies were carried out using a submillimeter high resolution (0.7 mm full width at half-maximum) small animal scanner (32 module quadHIDAC, Oxford Positron Systems Ltd., Oxford, UK) with uniform spatial resolution (<1 mm) over a large cylindrical field (165 mm diameter, 280 mm axial length). List-mode data were acquired for 90 min and reconstructed into dynamic time frames using an iterative reconstruction algorithm. In a subgroup of mice, dynamic measurements were complemented by late time point PET scans 4 hours post injection. Subsequently to PET acquisitions, the scanning bed was transferred to the computed tomography (CT) scanner (Inveon, Siemens Medical Solutions, U.S.), and a CT acquisition with a spatial resolution of 80 μm was performed for each mouse. Reconstructed image data sets were coregistered based on extrinsic markers attached to the multimodal scanning bed and the in-house developed image analysis software MEDgical. Three-dimensional volumes of interest (VOIs) were defined over the respective organs in CT data sets, transferred to the coregistered PET data, and analyzed quantitatively. Regional uptake was calculated as a percentage of injected dose by dividing counts per milliliter in the VOI by total counts in the mouse multiplied by 100 (% ID/mL).

Ex vivo gamma counter measurements. Following the final PET-CT acquisition 90 min p.i. or 250 min p.i. respectively, mice were euthanized by cervical dislocation and a necropsy was performed. Ex vivo biodistribution of radioactivity was analyzed by scintillation counting (Wizard2 gamma counter, Perkin-Elmer Life Science) and the radioactivity in respective organs decay-corrected and calculated as % ID per gram tissue (% ID/g).

### Preparation of Triethylammonium Hydrogencarbonate Buffer

A 1 M solution of TEAC was prepared by the following procedure. To a 1 M solution of triethylamine in deoinized water was added dry ice slowly until the pH value reached approximateley 8.4-8.6. The solution was stirred for several hours.¹

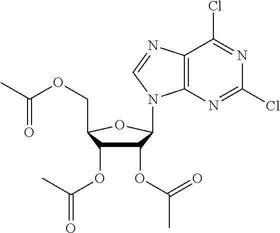

2,6-Dichloro-9-(2′,3′,5′-tri-O-acetyl-β-D-ribofuranosyl)-9H-purine (5). The reaction was carried out using previously published reaction conditions.¹³ 1,2,3,5-Tetraacetyl-β-D-ribofuranose (5 g, 16.0 mmol, 1.0 eq) was molten at 85° C., and 2,6-dichloropurine (3.0 g, 16.0 mmol, 1.0 eq) was added while stirring. Trifluoromethanesulfonic acid (70 μl, 0.8 mmol, 0.05 eq) was added to the reaction mixture in order to catalyze the reaction. The reaction mixture was stirred at 85° C. under reduced pressure for 30 min. Analysis by thin-layer chromatography (TLC) as performed to indicate completion of the reaction. The mixture was then cooled to rt Recrystallization from absolute ethanol yielded the desired product (4.84 g, yield 69%). ¹H NMR (600 MHz, DMSO-d₆) δ 8.90 (s, 1H, C8-H), 6.30 (d, 1H, J=4.9 Hz, C1′-H), 5.89 (q, 1H, J=5.4 Hz, C2′-H), 5.61 (t, 1H, J=5.6 Hz, C3′-H), 4.43-4.28 (d m, 2H, C5′-H₂), 4.38 (dd, 1H, J=3.6, 12.1 Hz, C4′-H), 2.10 (s, 3H, CH₃), 2.04 (s, 3H, CH₃), 2.00 (s, 3H, CH₃). ¹³C NMR (126 MHz, DMSO-d₆) δ 170.21, 169.55, 169.41, 152.96, 151.52, 150.47, 147.04, 13.43, 86.44, 80.03, 72.55, 69.94, 62.79, 20.65, 20.52, 20.38. LC-MS (m/z): positive mode 447.0 [M+H]⁺. Purity determined by HPLC-UV (254 nm)-ESI-MS: 88%. Mp: 160° C.

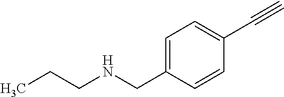

N-(4-Ethynylbenzyl)propan-1-amine (10). Compound 10 (N-(4-ethynylbenzyl)propan-1-amine) was synthesized according to the following reaction conditions. (4-Ethynylphenyl)methanamine (1, 0.5 g, 3.8 mmol, 1.0 eq) was reacted with 1-bromopropane (0.34 ml, 3.8 mmol, 1.0 eq) for two days at room temperature in methanol. The desired product precipitated in the the reaction mixture and was filtered off yielding 0.27 g of 10 (46%). ¹H NMR (600 MHz, DMSO-d₆) δ 8.77 (br s, 1H, NH), 7.53 (q, 2H, J=8.28 Hz, aryl), 4.26 (s, 1H, C—CH), 4.16 (s, 2H, NHCH₂-aryl), 2.85 (m, 2H, NHCH₂), 1.63 (m, 2H, CH₂CH₂CH₃), 0.89 (t, 3H, J=7.44 Hz, CH₃). ¹³C NMR (126 MHz, DMSO-d₆) δ 132.87, 132.03, 130.38, 122.43, 83.03, 81.86, 49.66, 48.36, 19.06, 11.05. LC-MS (m/z): positive mode 173.9 [M+H]⁺. Purity determined by HPLC-UV (254 nm)-ESI-MS: 95%.

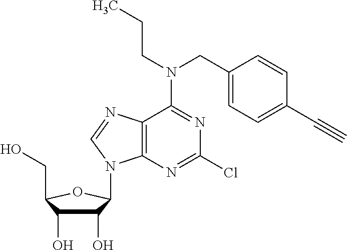

(2R,3R,4S,5R)-2-(2-Chloro-6-((4-ethynylbenzyl)propyl)amino)-9H-purin-9-yl)-5-(hydroxymethyl)tetrahydrofuran-3,4-diol (11). 2′,3′,5′-Tri-O-acetyl-2,6-dichlororibofuranosylpurine (5, 0.34 g, 0.77 mmol, 1.0 eq) was suspended in absolute ethanol. Triethylamine (0.2 ml, 1.54 mol, 2.0 eq) and N-(4-ethynylbenzyl)propan-1-amine 10 (0.26 g, 1.54 mmol, 2.0 eq) were added to the suspension which was refluxed overnight. The progress of the reaction was monitored by silica gel TLC (DCM/methanol 9/1). After TLC indicated completion of the reaction, the solvent was evaporated. The subsequent deprotection reaction was carried without further purification of 6 using 1 M sodium methoxide in methanol (10 ml). Purification by column chromatography (methanol/DCM 1:19) yielded the desired product as a white solid (0.25 g, 71%). ¹H NMR (600 MHz, DMSO-d₆) δ 8.42 (br s, 1H, C8-H), 7.42 (d, 2H, J=8.04 Hz, aryl), 7.28 (br s, 2H, aryl), 5.85 (d, J=5.83 Hz, 1H, C1′-H), 5.54 (d, 1H, J=12.20 Hz, 1× N—CH₂), 5.45 (br s, 1H, OH), 5.18 (br s, 1H, OH), 5.00 (br s, 1H, OH), 4.92 (d, 1H, J=15.05 Hz, 1×NCH₂), 4.51 (br s, 1H, C2′-H), 4.12 (s, 2H, NCH₂), 4.05 (q, 1H, J=5.19 Hz, C3′-H), 3.94 (q, 1H, J=3.92 Hz, C4′-H), 3.59 (dm, 2H, C5′-H₂), 1.63 (br s, 2H, CH₂ CH₃), 1.05 (t, 1H, J=7.02 Hz, alkyne-CH), 0.85 (t, 3H, CH₃). ¹³C NMR (126 MHz, CD₃OD) δ154.38, 152.79, 151.73, 139.27, 132.03, 127.78, 120.67, 118.46, 87.47, 85.85, 83.46, 80.83, 73.84, 70.48, 61.45, 39.72, 21.88, 18.60, 11.08. LC-MS (m/z): positive mode 458.2 [M+H]⁺. Purity determined by HPLC-UV (254 nm)-ESI-MS: 88%. Mp. 110° C.

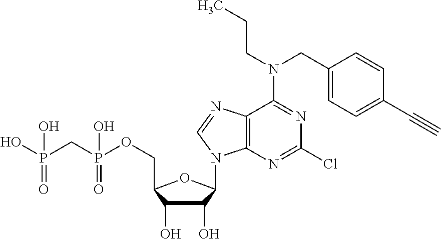

(((((2R,3S,4R,5R)-5-(2-Chloro-6-((4-ethynylbenzyl)propyl)amino)-9H-purin-9-yl)-3,4-dihydroxytetrahydrofuran-2-yl)methoxy)(hydroxy)phosphoryl)methyl)phosphonic acid (1). The phosphonylation reaction and the subsequent hydrolysis was performed by a previously reported method with modified reaction conditions.^{13-15, 16} (2R,3R,4S,5R)-2-(2-Chloro-6-((4-ethynylbenzyl)(propyl)amino)-9H-purin-9-yl)-5-(hydroxymethyl)tetrahydrofuran-3,4-diol (11, 0.1 g, 0.22 mmol, 1.0 eq.) was dissolved in trimethyl phosphate (2 ml) and stirred at 0-4° C. A solution of methylenebis(phosphonic dichloride) (0.27 g, 1.09 mmol. 5.0 eq.) in trimethyl phosphate (3 ml), cooled to 0-4° C. was added. The reaction mixture was stirred at 0-4° C. and samples were withdrawn at 15 min intervals for TLC to control for the disappearance of nucleosides. After 30 min, when the nucleoside had completely reacted, 20 ml of cold 0.5 M aqueous TEAC solution (pH 7.4-7.6) was added and the solution was stirred at 0° C. for 15 min followed by stirring at room temperature for 1 h. Trimethyl phosphate was extracted using 2×250 ml of tert-butyl methyl ether, and the aqueous layer was lyophylized. The crude product was then purified by RP-HPLC (0-50% MeCN/50 mM NH₄HCO₃ buffer within 20 min, 20 ml/min) and the appropriate fractions were pooled. The product was obtained as a white solid after lyophilization (0.11 g, 89%). ¹H NMR (600 MHz, D₂O) δ 8.37 (s, 1H, C8-H), 7.30 (br s, 2H, aryl), 7.18 (d, 2H, J=7.78 Hz, aryl), 6.01 (d, 1H, J=5.36 Hz, C1′-H), 5.31 (br s, 2H, NCH₂), 4.68 (t, 1H, J=5.20 Hz, C2′-H), 4.51 (d, 1H, J=4.70 Hz, C3′-H), 4.36 (d, 1H, J=3.79 Hz, C4′-H), 4.16 (t, 2H, J=4.24 Hz, C5′-H₂), 3.92 (br s, 2H, NCH₂), 3.43 (s, 1H, alkyne-CH), 2.19 (t, 2H, J=19.68 Hz, PCH₂P), 1.62 (br s, 2H, CH₂CH₃), 0.84 (t, 3H, J=7.55 Hz, CH₃). ¹³C NMR (126 MHz, D₂O) δ 167.45, 157.58, 156.41, 154.05, 141.25, 141.05, 134.96, 130.31, 130.06, 125.70, 123.06, 120.82, 89.78, 86.57, 85.91, 81.00, 77.15, 72.99, 66.38, 53.53, 30.35, 23.86, 13.11. ³¹P NMR (202 MHz, D₂O) δ 18.95 (d, 1P, J=9.50 Hz, P_{β}) 14.95 (d, 1P, J=9.28 Hz, P_{α}). LC/ESI-MS (m/z): positive mode 616.1130 [M+H]⁺ (calcd. for C₂₃H₂₉ClN₅O₉P₂ 616.1129) and negative mode 614.0991 [M−H]⁻ (calcd. for C23H₂₇ClN₅O₉P₂ 614.0973). Purity determined by HPLC-UV (254 nm)-ESI-MS: 84%. Mp. 141° C.

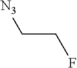

1-Azido-2-fluoroethane (8). 2-Fluoroethyl-4-toluenesulfonate (7, 0.05 ml, 0.3 mmol, 1.0 eq.) was dissolved in anhydrous DMF (0.5 ml). Sodium azide (0.05 g, 0.9 mmol, 3.0 eq.) was added. The reaction was stirred at rt for 24 h and monitored by TLC. The crude mixture was filtered, and the filtrate was used without further purification. WARNING: Attempts to isolate neat 1-azido-2-fluoroethane may result in explosion.

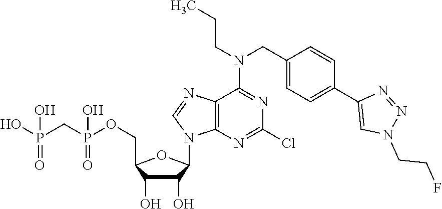

(((((2R,3S,4R,5R)-5-(2-Chloro-6-((4-(1-(2-fluoroethyl)-1H-1,2,3-triazol-4-yl)benzyl(propyl)amino)-9H-purin-9-yl)-3,4-dihydroxytetrahydrofuran-2-yl)methoxy)(hydroxy)phosphoryl)methyl)phosphonic acid (2). (((((2R,3S,4R,5R)-5-(2-Chloro-6-((4-ethynylbenzyl)(propyl)amino)-9H-purin-9-yl)-3,4-dihydroxytetrahydrofuran-2-yl)methoxy)(hydroxy)phosphoryl)methyl)phosphonic acid 1 (15.0 mg, 0.02 mmol, 1.0 eq) was dissolved in THF/H₂O/t-BuOH (3:1:1, 0.5 ml). Crude 2-fluoroethylazide 8 (50.0 μl) was added. Additionally, 1 M sodium ascorbate in H₂O (0.03 ml, 0.03 mmol, 1.2 eq) was added. Finally, a premixed solution of CuSO₄ (1.0 mg, 0.009 mmol, 0.3 eq) and TBTA (4.0 mg, 0.003 mmol, 0.3 eq) in THF/H₂O/t-BuOH (3:1:1, 0.5 ml) was added to the reaction mixture. The reaction was stirred in the dark under argon at rt. During the reaction, the color of the reaction mixture changed from a bright yellow to milky mint green. After one night of stirring the reaction mixture was evaporated and directly purified by RP-HPLC (0-70% MeCN/50 mM NH₄HCO₃ buffer within 20 min, 20 ml/min). Appropriate fractions were pooled and lyophilyzed overnight to obtain the final product as a white solid (0.003 g, 17.5%). ¹H NMR (600 MHz, D₂O) δ 8.40 (s, 1H, triazolyl), 8.27 (s, 1H, C8-H), 7.67 (d, 2H, J=6.91 Hz, aryl), 7.36 (d, 2H, J=7.90 Hz, aryl), 6.02 (d, 1H, J=4.74 Hz, C1′-H), 4.93-4.85 (m, 2H, NCH₂), 4.77 (m, 2H, overlapping with H₂O:NCH₂), 4.73 (s, 1H, C2′-H), 4.53 (s, 1H, C3′-H), 4.38 (s, 1H, C4′-H), 4.18 (br s, 2H, C5′-H₂), 3.15 (m, 2H, triazole-NCH₂), 2.18 (br s, 2H, PCH₂P), 1.67 (m, 2H, CH₂F), 1.34 (m, 2H, CH₂CH₃), 0.91 (m, 3H, CH₃). ¹³C NMR (126 MHz, D₂O) δ 159.10, 156.48, 154.08, 152.57, 143.88, 141.04, 131.31, 130.98, 128.64, 125.38, 120.81, 89.68, 86.75, 85.48, 77.08, 73.07, 66.51, 55.52, 53.76, 53.63, 49.54, 28.06, 15.63. ³¹P NMR (202 MHz, D₂O) δ 16.44 (s, 1P, P_{β}) 0.39 (s, 1P, P_{α}). ¹⁹F NMR (565 MHz, D₂O) δ −75.62 Hz. LC/ESI-MS (m/z): positive mode 705.1486 [M+H]⁺ (calcd. for C₂₅H₃₃ClFN₈O₉P₂ 705.1518) and negative mode 703.1335 [M−H]⁻ (calcd. C₂₅H₃₁ClFN₈O₉P₂ 703.1362). Purity determined by HPLC-UV (254 nm)-ESI-MS: 99%. Mp. 169° C. Estimated log P²⁷ 3.3, estimated clogP: 0.22.

### (2R,3R,4S,5R)-2-(2-Chloro-6-((4-(1-(2-fluoroethyl)-1H-1,2,3-triazol-4-yl)benzyl)(propyl)amino)-9H-purin-9-yl)-5-(hydroxymethyl)tetrahydrofuran-3,4-diol (12)

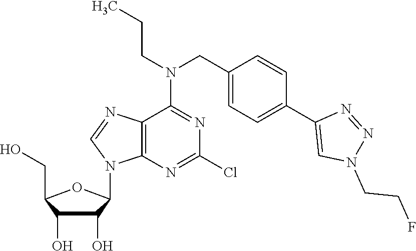

(2R,3R,4S,5R)-2-(2-Chloro-6-((4-ethynylbenzyl)(propyl)amino)-9H-purin-9-yl)-5-(hydroxymethyl)tetrahydrofuran-3,4-diol (11, 0.080 g, 0.017 mmol, 1.0 eq.) was dissolved in a solution of 8 in DMF (5 ml). TBTA (0.028 g, 0.052 mmol, 0.3 eq.) was added to the reaction mixture. Copper sulfate (0.008 g, 0.052 mmol, 0.3 eq.) and sodium ascorbate (0.041 g, 0.21 mmol, 1.2 eq.) were premixed in 2 ml of water and then added to the reaction mixture, which was then stirred overnight. After TLC indicated completion of the reaction, the reaction mixture was diluted with water and extracted with ethyl acetate followed by washing with an aqueous lithium chloride solution (10%). The organic phase was dried of sodium sulfate, evaporated and then purified by normal phase column chromatography (silica gel, DCM/methanol 95/5). Appropriate fractions were pooled and the eluents were evaporated to give the desired compound as colorless solid (yield: 76%, 0.073 g). Mp. 180-183° C. ¹H NMR (600 MHz, DMSO-d₆) δ 8.55 (s, 1H, triazolyl), 8.49-8.36 (m, 1H, C8-H), 7.80 (d, J=7.0 Hz, 2H, aryl), 7.42-7.31 (m, 2H, aryl), 5.86 (d, J=5.8 Hz, 1H, C1′-H), 5.57 (d, J=20.9 Hz, 1H, 1×N—CH₂), 5.49 (s, 1H, OH), 5.20 (s, 1H, OH), 5.03 (s, 1H, OH), 4.95 (d, J=21.2 Hz, 1H, 1×N—CH₂), 4.89 (t, J=4.7 Hz, 1H, F—CH₂—CH₂), 4.81 (t, J=4.7 Hz, 1H, F—CH₂—CH₂), 4.76 (t, J=4.7 Hz, 1H, F—CH₂), 4.72 (t, J=4.7 Hz, 1H, F—CH₂), 4.52 (t, J=5.3 Hz, 1H, C2′-H), 4.15-4.11 (m, 1H, C3′-H), 3.94 (q, J=3.8 Hz, 1H, C4′-H), 3.65 (d, J=11.9 Hz, 1H, C5′-H), 3.55 (d, J=12.2 Hz, 1H, C5′-H), 1.71-1.57 (m, 2H, CH₂CH₃), 0.90-0.82 (m, 3H, CH₃). ¹³C NMR (151 MHz, DMSO-d₆) δ 162.45, 154.49, 154.39, 152.86, 152.73, 151.73, 146.43, 137.88, 137.41, 129.83, 128.26, 128.02, 125.54, 121.88, 118.47, 118.37, 87.46, 85.85, 82.57, 81.45, 73.84, 70.48, 61.44, 50.41, 50.28, 35.92, 30.92, 21.37, 19.86, 11.36, 10.89. ¹⁹F NMR (565 MHz, DMSO-d6) δ −74.22. LC/ESI-MS (m/z): positive mode 547.40 [M+H]⁺ Purity determined by HPLC-UV (254 nm)-ESI-MS: 98%. Mp. 180-183° C.

### [¹⁸F]-[((2R,3S,4R,5R)-5-{2-chloro-6-[(4-{1-[2-(fluoro)ethyl]-1H-1,2,3-triazol-4-yl}benzyl)(propyl)amino]-9H-purin-9-yl}-3,4-dihydroxytetrahydrofuran-2-yl)methoxy]methylenebisphosphonic acid ([¹⁸F]2)

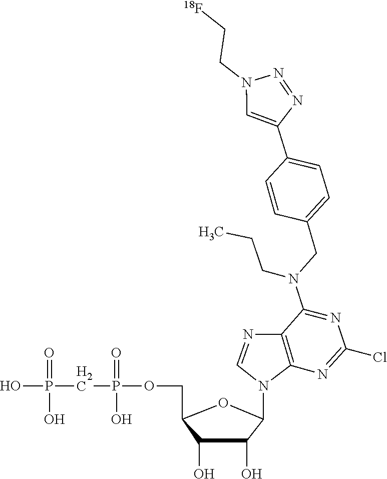

In a computer controlled TRACERLab Fx_{FDG} Synthesizer a batch of aqueous [¹⁸F]F-ions (3.1-5.2 GBq) from the cyclotron target was passed through an anion exchange resin (pre-conditioned Sep-Pak® Light QMA cartridge with carbonate counter-ion). [¹⁸F]F-ions were eluted from the resin with a mixture of 1 M K₂CO₃ (aqueous, 40 μL), water for injection (WFI, 200 μL), and acetonitrile (800 μL, DNA-grade) containing Kryptofix® 2.2.2 (K_{2.2.2}, 20 mg, 53 μmol) in the reactor. Subsequently, the aqueous K(K_{2.2.2})[¹⁸F]F solution was carefully evaporated to dryness in vacuo. An amount of precursor compound 2-azidoethyl 4-methylbenzenesulfonate (20 mg, 83 μmol) in acetonitrile (DNA-grade, 500 μL) was added and the mixture was heated at 110° C. for 3 min. Meanwhile, the labeled 1-azido-2-[¹⁸F]fluoroethane was distilled from the reactor into an ice-cooled 10 mL flask that contained a mixture of PSB-19425 (1, 5.0 mg, 8.1 μmol) in DMF (300 μL), CuSO₄·5H₂O (40 mg, 160 μmol) in HEPES buffer (pH: 5.7, 100 μL) and sodium ascorbate (63 mg, 318 μmol) in HEPES buffer (pH: 5.7, 100 μL). After 30 min stirring at 60° C., the mixture was passed through a PTFE sterile filter (0.2 μm). The filter was rinsed with DMF (500 μL) and then with WFI (500 μL). The combined filtrate and rinsing solutions were purified by gradient-radio-HPLC system A (method A). The product fraction of compound [¹⁸F]PSB-19427 (retention time t_{R}([¹⁸F]PSB-19427)=12.2 min) was collected in a flask pre-treated with Sigmacote® and the solution was evaporated to dryness in vacuo. The residue was redissolved in water for injection (WFI)/EtOH (1 mL, 9:1 v/v). Product compound [¹⁸F]PSB-19427 was obtained in an overall radiochemical yield of 21.2±3.0% (decay-corrected, based on cyclotron-derived [¹⁸F]F-ions, n=20) in 119±10 min from the end of radionuclide production. [¹⁸F]PSB-19427 was isolated in radiochemical purities of >99% with molar activities in the range of 2.3-176.6 GBq/μmol at the end of the synthesis. Radiochemical purities and molar activities of [¹⁸F]PSB-19427 (retention time t_{R}([¹⁸F]PSB-19427)=9.4 min) were determined by analytical radio-HPLC B (method B).

### In Vitro Stability in Mouse and Human Serum.

The serum stability of the radioligand [¹⁸F]PSB-19427 was evaluated by incubation in mouse serum at 37° C. for up to 90 min. An aliquot of formulated [¹⁸F]PSB-19427 (20 μL, 3.9 MBq) was added to a sample of mouse serum (200 μL), and the mixture was incubated at 37° C. Samples of 20 μL each were drawn after periods of 10, 30, 60 and 90 min and quenched in ice-cold acetonitrile (100 μL, DNA-grade) followed by centrifugation (3000 rpm) for ≥5 min. The supernatant was analyzed by analytical radio-HPLC B (method B). Serum stability investigations in human serum were performed analogously.

Determination of the log D_{7,4}-value of [¹⁸F]PSB-19427. The lipophilicity of [¹⁸F]PSB-19427 was determined following the procedure described by Prante et al.²⁸ Triazole [¹⁸F]PSB-19427 (˜400 kBq) in PBS buffer (10 μL, pH 7.4) was added to PBS buffer (590 μL, pH 7.4) and octan-1-ol (600 μL). The two-layer mixture was shaken for 3 min on a vortex mixer at RT and centrifuged (3000 rpm) for 5 min. The main part of the octanol layer (400 μL) was collected carefully and added to a new tube with PBS buffer (400 μL, pH 7.4). The two-layer mixture was shaken again for 10 min and centrifuged for 5 min (3000 rpm). Three samples were prepared and two aliquots (á 100 μL) of both layers were measured in a g-counter 2480 Wizard2 (Perkin-Elmer, Waltham, USA). The partition coefficient was determined by dividing cpm(octanol) by cpm(PBS) and indicated as log D_{7.4} (exp.).

Partition coefficient (log D (exp.)). [¹⁸F]PSB-19427 is a hydrophilic compound with a log D (exp.) of −0.12±0.03. Compared to the calculated log P values (clog P) of 0.22¹³ and 0.47¹⁵ the measured solubility of [¹⁸F]PSB-19427 in PBS-buffer in the biphasic 1-octanol/PBS system is increased by factor 2.2 and 3.9, respectively.

Determination of plasma protein binding (PPB) was done following the procedure of Börgel et al.¹³

For the quantification of different analytes, a LC system was coupled with a single quadrupole (SQ) mass spectrometer.

UPLC-UV/MS (Agilent, Waldbronn, Germany): pump: 1260 Bin Pump (G1212B); degasser: 1260 HiP (G4225A); column oven: 1290 TCC (G1316C), 30° C.; autosampler 1260 HiP ALS (G1367E), 1 □L injected if not stated otherwise; UV/vis detector 1260 VWD (G1314F); MS source: multimode source (G1978B); MS-Detector 6120 Quadrupole (G1978B); MS parameters: Vaporizer temperature: 250° C.; drying gas: 10 L/min; nebulizer pressure: 40 psi; capillary voltage: 3000 V; fragmentor voltage: 100 V; drying gas temperature: 350° C.; LC parameters: precolumn: Chiralpak® HSA HPLC Guard Column (2.0×10 mm, 5 μm particle size); main column: Chiralpak® HSA HPLC Column (2.0×50 mm, 5 μm particle size, Daicel, Eschborn, Germany); temperature: 25° C.; mobile phase: aqueous ammonium acetate solution (50 mM, pH 7.4)/i-propanol 96:4; flow rate: 0.3 mL/min; isocratic.

Reference compounds and PSB-19427 were dissolved in methanol, and retention times were measured in LC-MS. D-glucose was used to determine the dead time (0.75 min) of the system. Reference compounds were used to plot k′/(k′+1) against known PPB values (Table 1, Error! Reference source not found.).

$${k}^{′} = \frac{{t}_{R} - {t}_{D}}{{t}_{D}}$$

With k′=retention factor
   t_{R}=retention time [min]
   t_{D}=death time [min]

TABLE 2
Measured retention times and known
PPB values of reference compounds.

| Compound | t_{R} (n = 3) | k′ | k′/(k′ + 1) | PPB [%] |
|---|---|---|---|---|
| Metronidazol | 0.87 | 0.16 | 0.14 | 11.0²⁹ |
| Paracetamol | 0.99 | 0.33 | 0.25 | 24.0³⁰ |
| Salbutamol | 1.10 | 0.47 | 0.32 | 7.5³⁰ |
| Sulfamethoxazol | 1.32 | 0.77 | 0.43 | 66.0²⁹ |
| Ramipril | 1.54 | 1.06 | 0.51 | 56.0²⁹ |
| Phenytoin | 3.83 | 4.12 | 0.80 | 91.0²⁹ |
| Propranolol | 4.05 | 4.42 | 0.82 | 87.0²⁹ |
| Imipramine | 8.35 | 10.17 | 0.91 | 92.6²⁹ |
| Haloperidol | 8.83 | 10.80 | 0.92 | 92.0²⁹ |
| Chlorpromazine | 33.51 | 43.82 | 0.98 | 97.8²⁹ |

### Chemistry—Synthesis of Fluorescein-Labeled CD73 Inhibitor

5(6)-Carboxyfluorescein (21) was attached to commercially available N-boc-1,6-hexanediamine via amide coupling using O-(7-azabenzotriazol-1-yl)-N,N,N′,N′-tetramethyluronium-hexafluorphosphat (HATU) as a coupling reagent, and N,N-diisopropylethylamine (DIPEA) as a base in DMF to give 22.¹⁹ Subsequent boo-deprotection using trifluoroacetic acid (TFA) in dichloromethane (DCM) yielded 23.¹⁸ Next, 4-(boo-aminobenzyl)benzoic acid was coupled to the primary amino group of 23 again employing HATU and DIPEA to give 24, followed by boc-deprotection with TFA in DCM yielding 28.^{18, 19}

To obtain the 2-bromo-6-chloro-9-(β-D-ribofuranosyl)purine (26), the commercially available 2-amino-6-chloro-9-(β-D-ribofuranosyl)-9H-purine (25) was reacted by a non-aqueous diazotization reaction using tert-butyl nitrite (TBN) and trimethylsilybromide (TMSBr) in dibromomethane.^{31, 32}

Then 2-bromo-6-chloro-9-(β-D-ribofuranosyl)purine (26) was phosphonylated using methylenbis(phosphonic dichloride) in trimethyl phosphate, followed by quenching with aqueous ammonium bicarbonate buffer.^{13, 18}

RP-HPLC purification afforded the pure product 27. In the final reaction step, the nucleophilic substitution of 27 by the primary amine 28 in absolute ethanol in the presence of triethylamine under reflux conditions, followed by RP-HPLC purification, yielded the desired final product 20 (yield 8%).¹⁸ The structure of 29 was confirmed by ¹H, ¹³C and ³¹P-NMR spectroscopy and LC/ESI-(UV)MS analysis (positive and negative mode) indicating a purity of >95%.

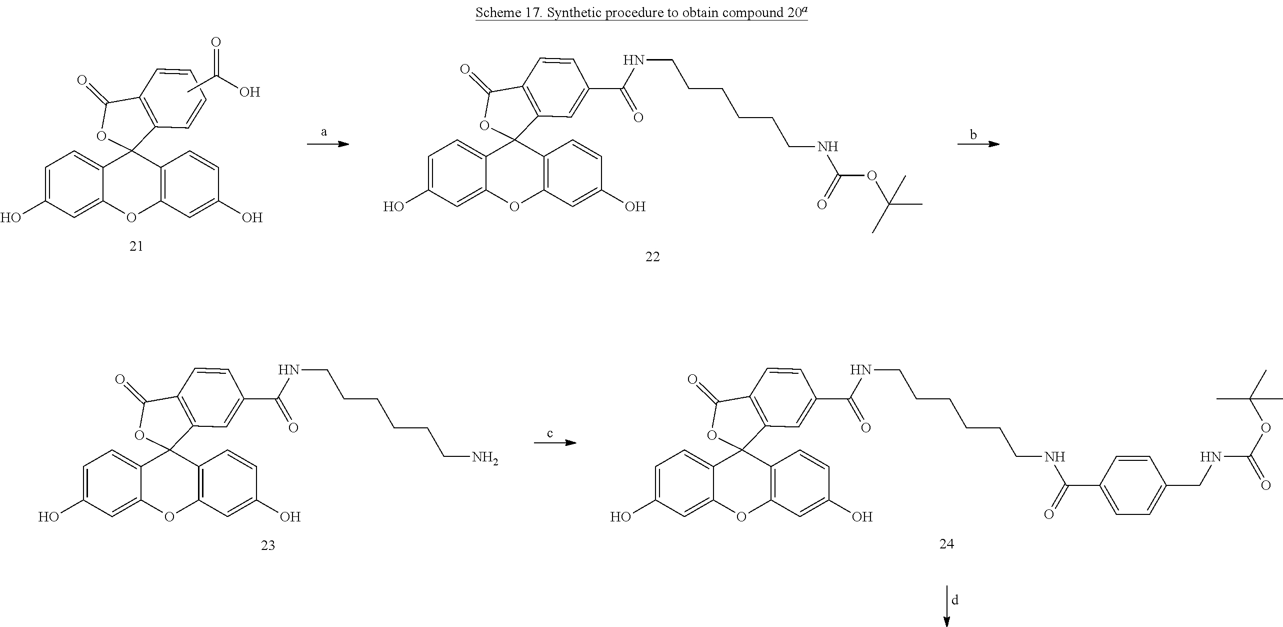

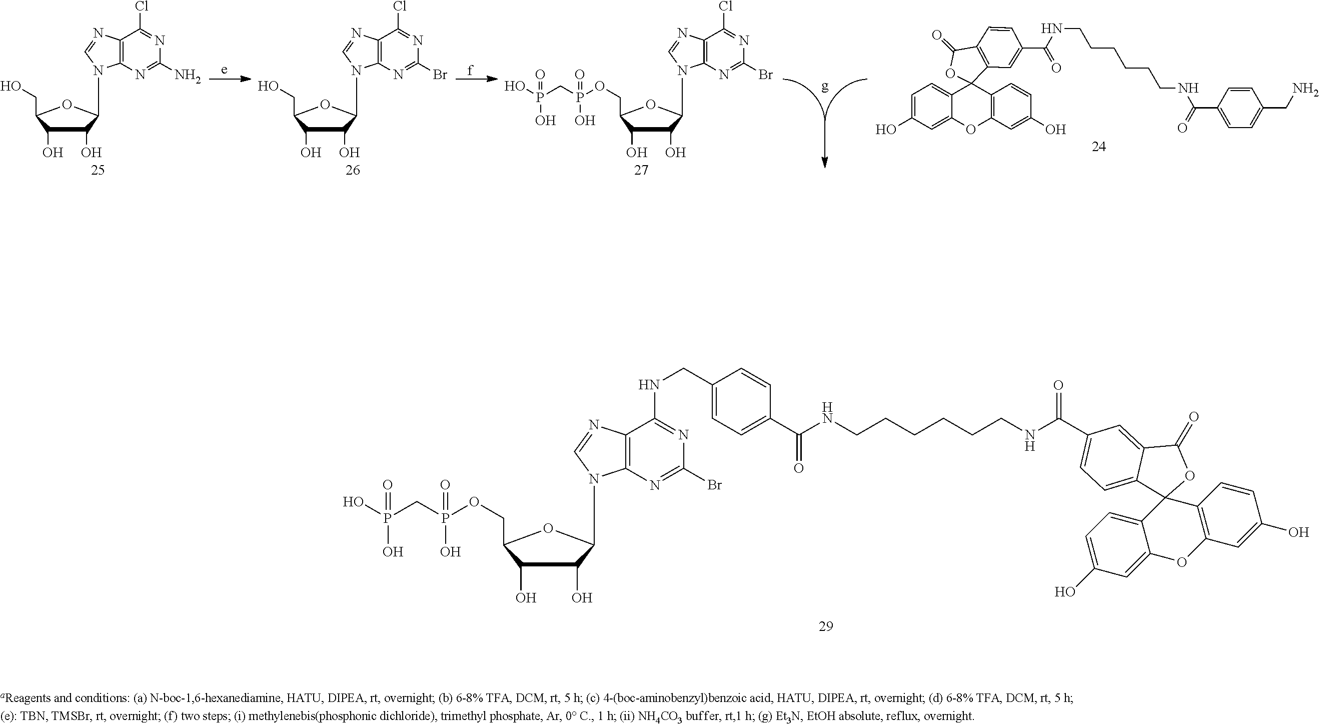

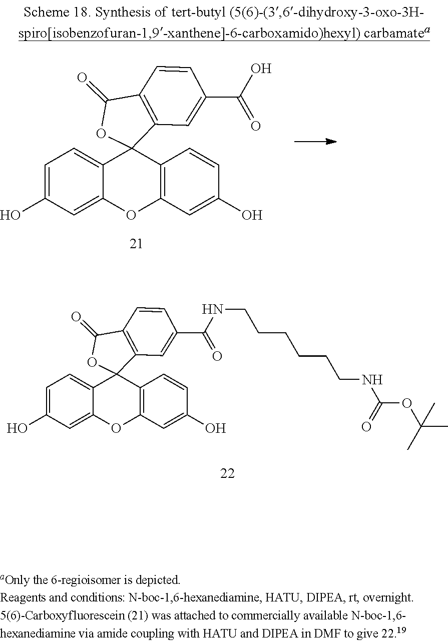 [SMILES: O=C(O)c1ccc2c(c1)C1(OC2=O)c2ccc(O)cc2Oc2cc(O)ccc21 ; CC(C)(C)OC(=O)NCCCCCCNC(=O)c1ccc2c(c1)C1(OC2=O)c2ccc(O)cc2Oc2cc(O)ccc21]

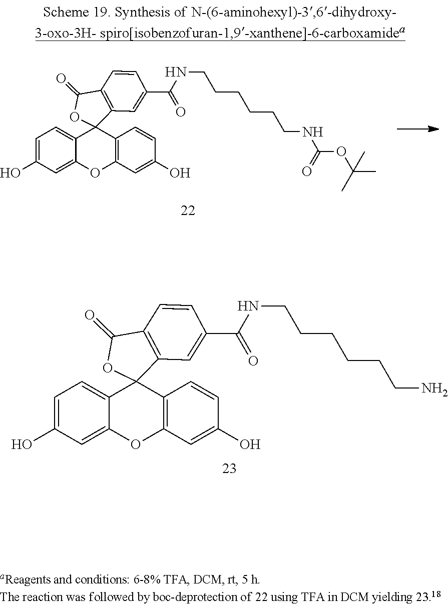 [SMILES: CC(C)(C)OC(=O)NCCCCCCNC(=O)c1ccc2c(c1)C1(OC2=O)c2ccc(O)cc2Oc2cc(O)ccc21 ; NCCCCCCNC(=O)c1ccc2c(c1)C1(OC2=O)c2ccc(O)cc2Oc2cc(O)ccc21]

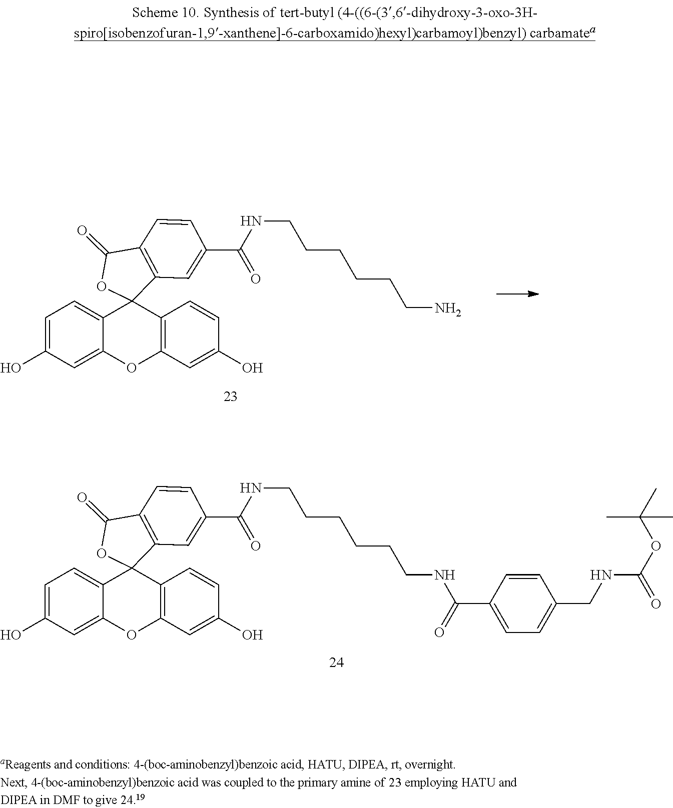 [SMILES: NCCCCCCNC(=O)c1ccc2c(c1)C1(OC2=O)c2ccc(O)cc2Oc2cc(O)ccc21 ; CC(C)(C)OC(=O)NCc1ccc(C(=O)NCCCCCCNC(=O)c2ccc3c(c2)C2(OC3=O)c3ccc(O)cc3Oc3cc(O)ccc32)cc1]

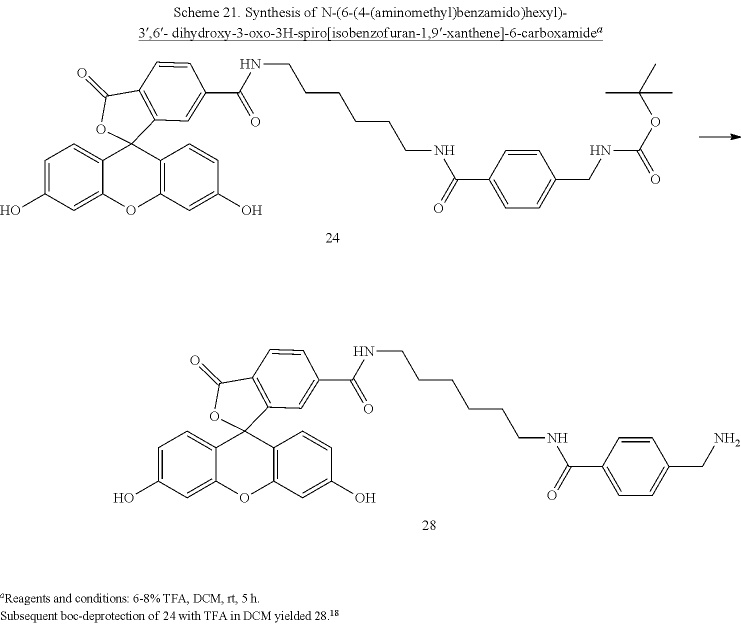 [SMILES: CC(C)(C)OC(=O)NCc1ccc(C(=O)NCCCCCCNC(=O)c2ccc3c(c2)C2(OC3=O)c3ccc(O)cc3Oc3cc(O)ccc32)cc1 ; NCc1ccc(C(=O)NCCCCCCNC(=O)c2ccc3c(c2)C2(OC3=O)c3ccc(O)cc3Oc3cc(O)ccc32)cc1]

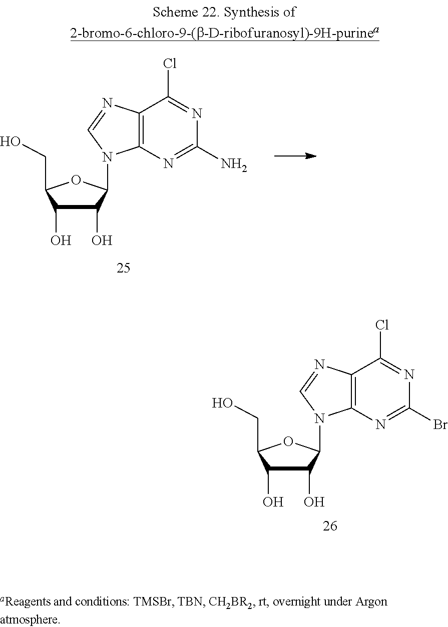

Commercially availabe 2-amino-6-chloro-9-(β-D-ribofuranosyl)purine (25) was reacted by a non-aqueous diazotization reaction using tert-butyl nitrite (TBN) and trimethylsilybromide (TMSBr) in dibromomethane. The reaction was carried out according to an altered previously published method to affording 26.^{31, 32}

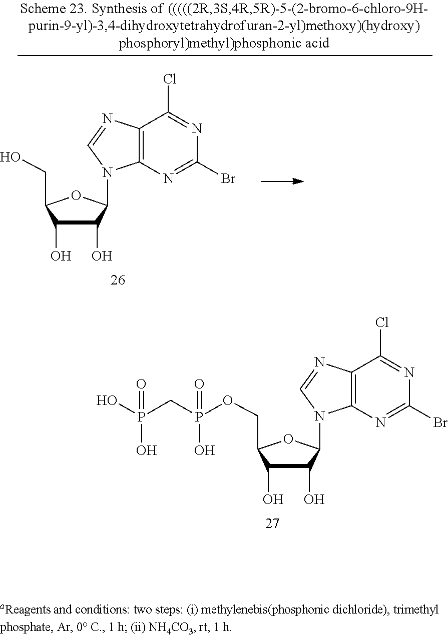

2-bromo-6-chloro-9-(-D-ribofuranosyl)-9H-purine (26) was phosphonylated using methylenbis(phosphonic dichloride) in trimethyl phosphate, followed by quenching with aqueous TEAC buffer. Both reaction steps were carried out according to an optimized previously published method affording 27.¹³

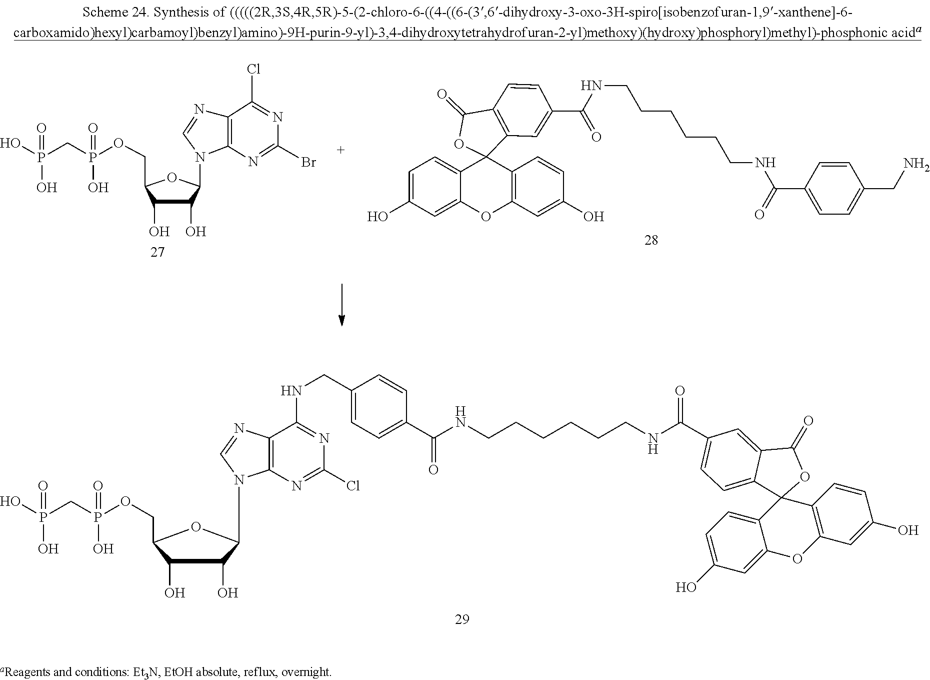

In the final reaction step, nucleophilic substitution of 27 by the primary amine 28 in absolute ethanol in the presence of triethyl amine under reflux conditions, followed by RP-HPLC purification, yielded the desired final product 29 (yield 8%).¹⁸

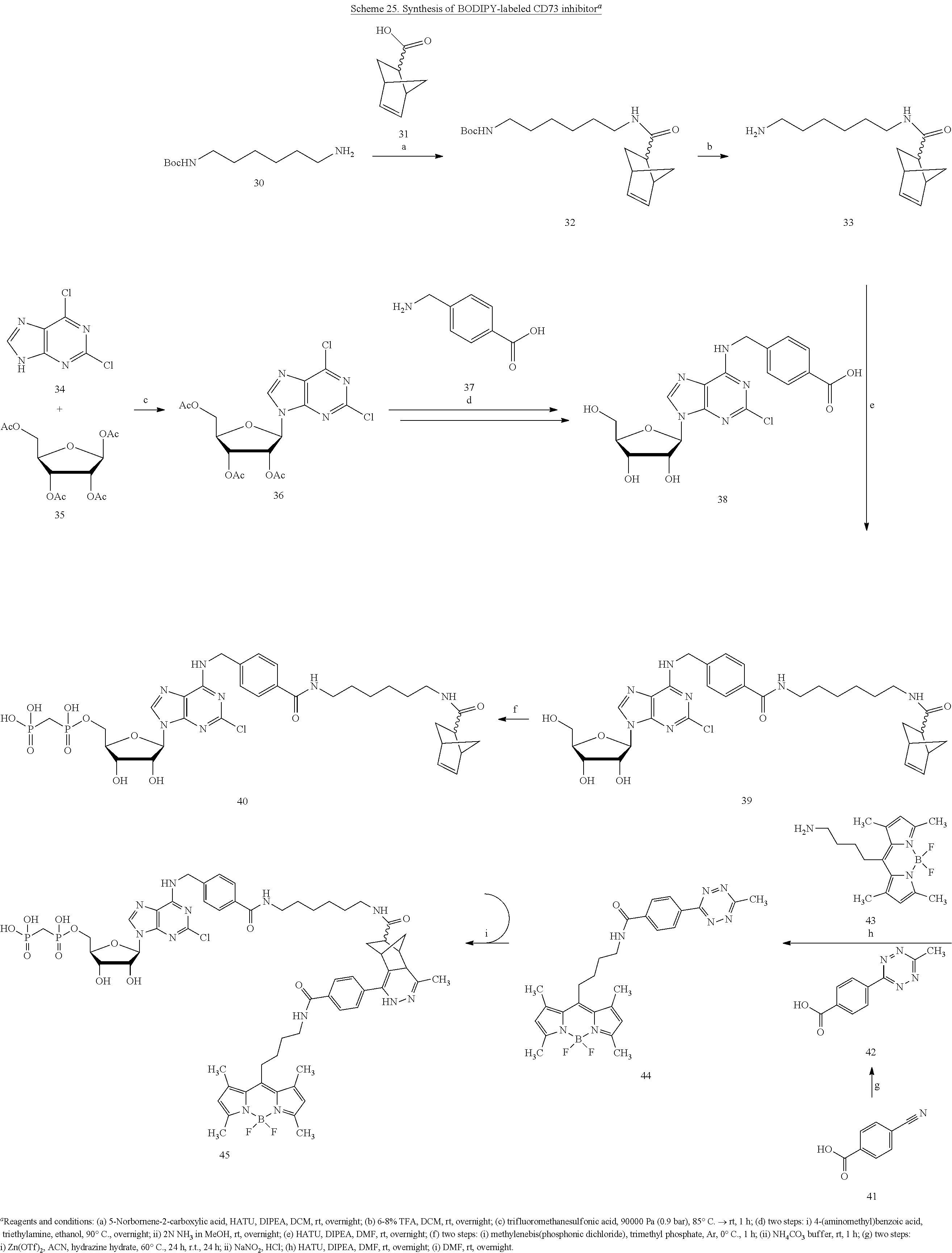

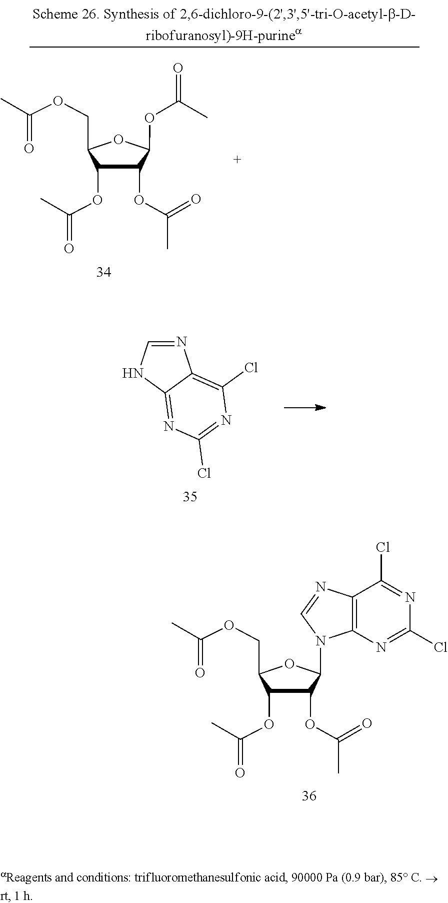

2,6-Dichloro-9-(2′,3′,5′-tri-O-acetyl-β-D-ribofuranosyl)-9H-purine (36) was synthesized starting from 1,2,3,5-tetraacetyl-β-D-ribofuranose (34) and 2,6-dichloropurine (35). The reaction was carried out using previously published reaction conditions.¹³ Both compounds were melted at 85° C. and reacted after the addition of trifluoromethanesulfonic acid.

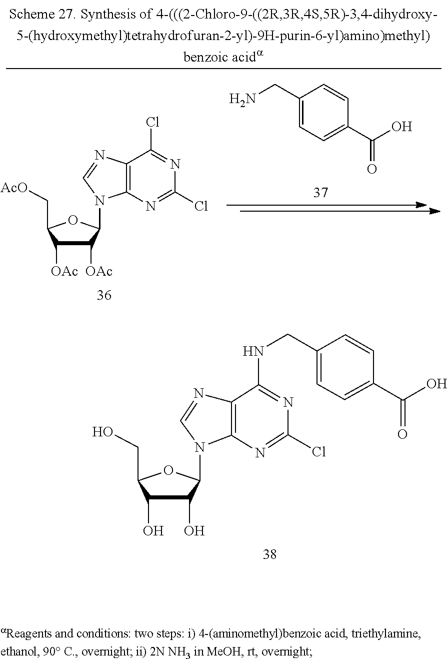

2′,3′,5′-Tri-O-acetyl-2,6-dichloropurine riboside (36) was reacted with 4-(aminomethyl)benzoic acid (37) under basic conditions in the presence of triethylamine. The mixture was then refluxed at 90° C. overnight.¹⁸ After TLC indicated the completion of the reaction the volatiles were evaporated, 2N NH₃ in MeOH was given to the crude product and the mixture was stirred at room temperature overnight. After evaporation of the solvent, ethanol abs. was given to the flask and heated to 100° C. to recrystallized the unreacted 4-(aminomethyl)benzoic acid. After filtration the volatiles were evaporated and 38 could be crystallized from methanol.

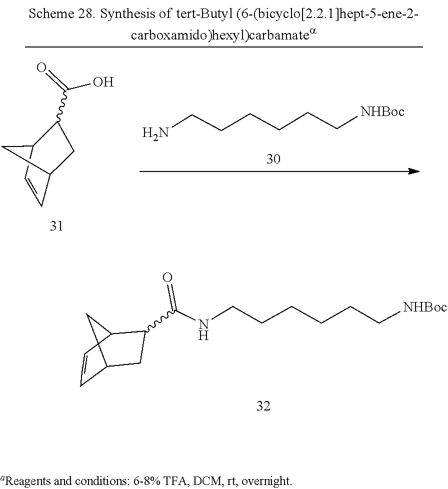 [SMILES: O=C(O)C1CC2CC=CC1C2 ; CC(C)(C)OC(=O)NCCCCCCN ; CC(C)(C)OC(=O)NCCCCCCNC(=O)C1CC2CC=CC1C2]

A mixture of endo- and exo-isomers of 5-norbornene-2-carboxylic acid (31) was attached to commercially available N-boc-1,6-hexanediamine (30) via amide coupling with HATU and DIPEA in DCM to give 32.¹⁹

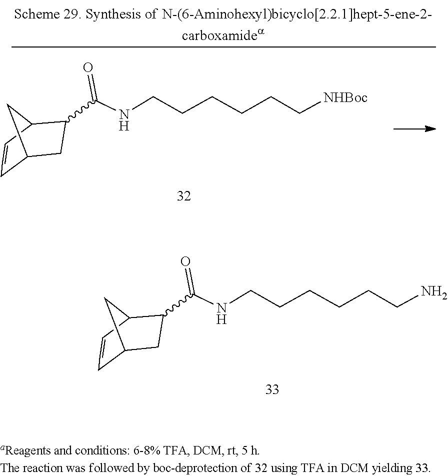 [SMILES: CC(C)(C)OC(=O)NCCCCCCNC(=O)C1CC2CC=CC1C2 ; NCCCCCCNC(=O)C1CC2CC=CC1C2]

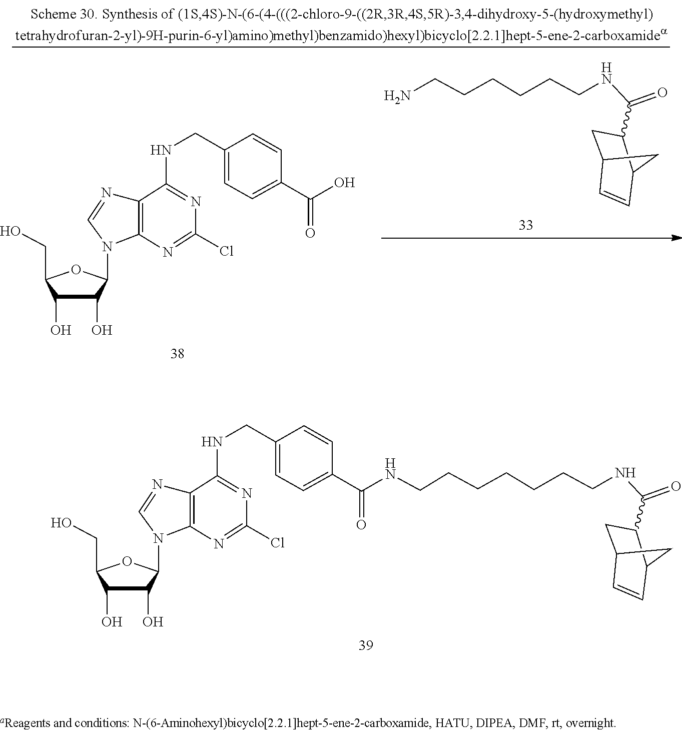

N-(6-Aminohexyl)bicyclo[2.2.1]hept-5-ene-2-carboxamide (33) was attached to 4-(((2-Chloro-9-((2R,3R,4S,5R)-3,4-dihydroxy-5-(hydroxymethyl)tetrahydrofuran-2-yl)-9H-purin-6-yl)amino)methyl)benzoic acid (38) via an amide coupling reaction to give 39.¹⁹

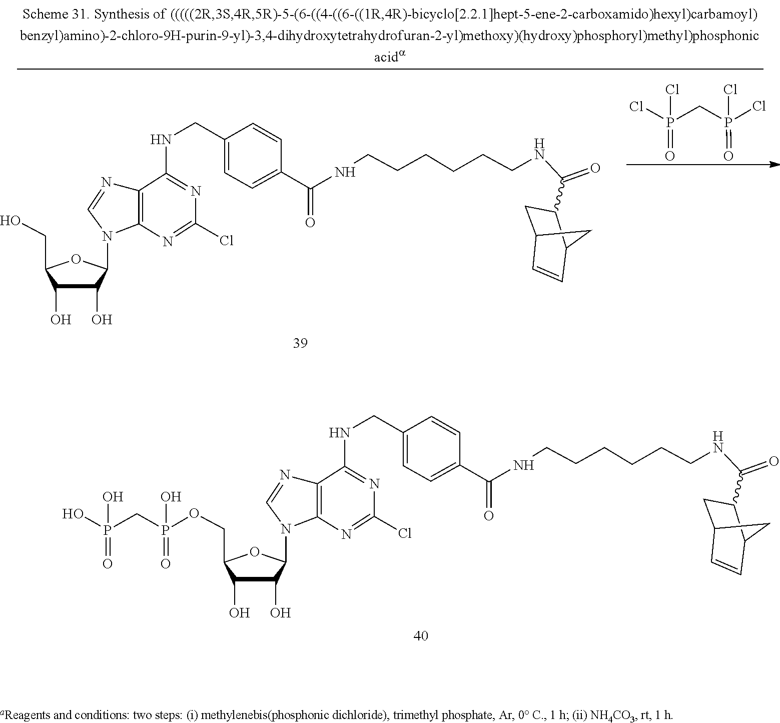

(1 S,4S)—N-(6-(4-(((2-chloro-9-((2R,3R,4S,5R)-3,4-dihydroxy-5-(hydroxymethyl)tetrahydrofuran-2-yl)-9H-purin-6-yl)amino)methyl)benzamido)hexyl)bicyclo[2.2.1]hept-5-ene-2-carboxamide (39) was phosphonylated using methylenbis(phosphonic dichloride) in trimethyl phosphate, followed by quenching with aqueous ammonium bicarbonate buffer. Both reaction steps were carried out according to an optimized previously published method affording 40.^{13, 18}

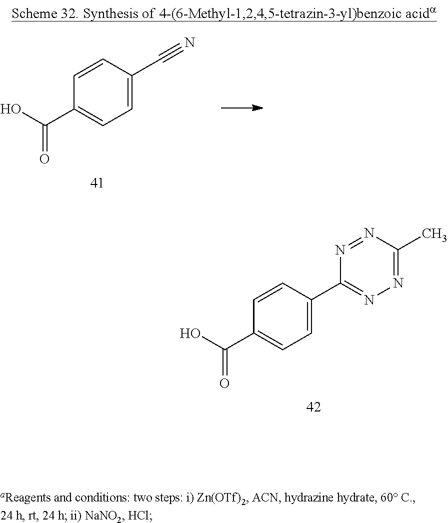 [SMILES: N#Cc1ccc(C(=O)O)cc1 ; Cc1nnc(-c2ccc(C(=O)O)cc2)nn1]

4-Cyanobenzoic acid (41), Zn(OTf)₂ and acetonitrile were added to a solution of hydrazine hydrate 80% w/w, stirred at 60° C. for 24 h and then at rt for another 24 h. Then an aqueous NaNO₂ solution was added slowly followed by conc. HCl until pH 3 is reached and gas evolution is ceased to give 42.³³

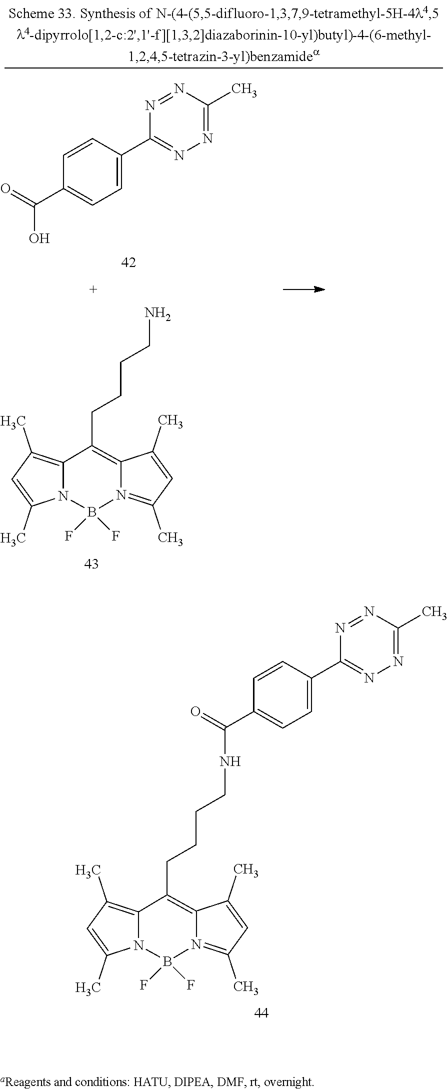

4-(6-Methyl-1,2,4,5-tetrazin-3-yl)benzoic acid (42) was attached to 4-(5,5-difluoro-1,3,7,9-tetramethyl-5H-4λ⁴,5λ⁴-dipyrrolo[1,2-c:2′,1′-f][1,3,2]diazaborinin-10-yl)butan-1-amine (43) via an amide coupling reaction to give 44.

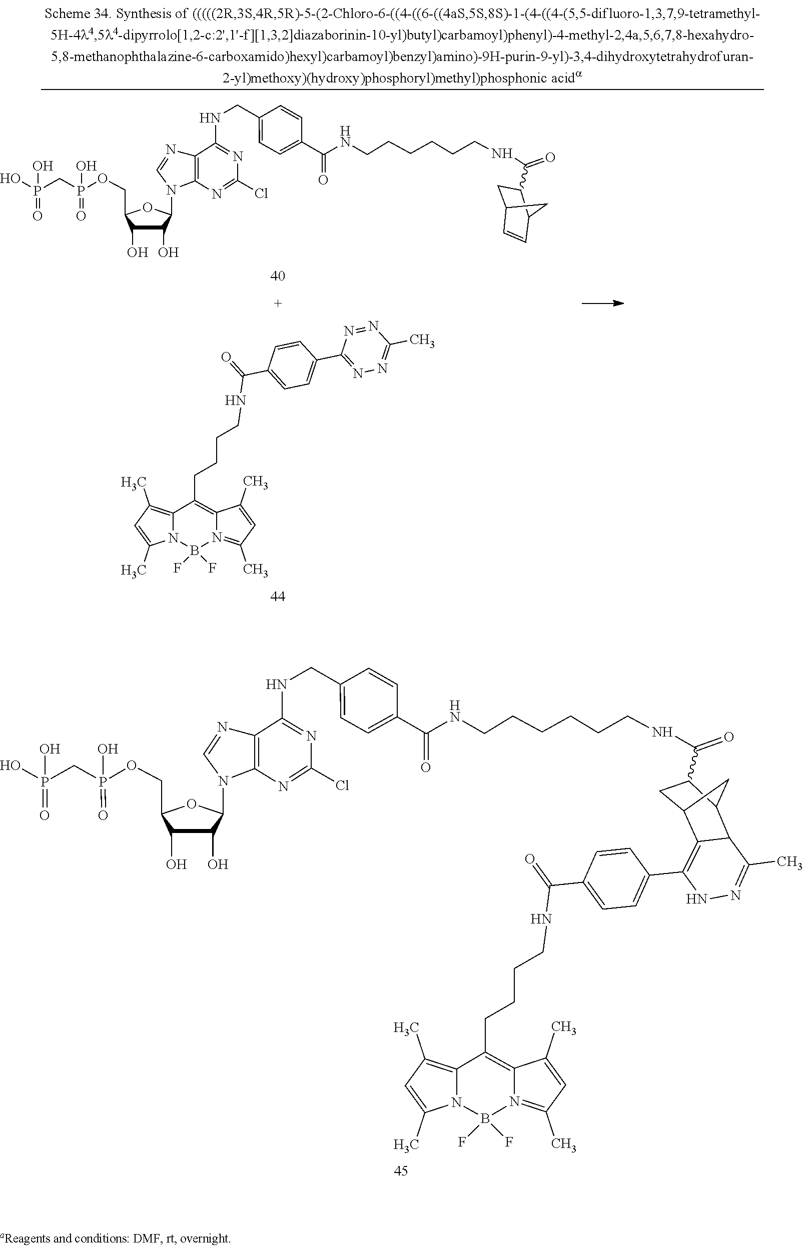

(((((2R,3S,4R,5R)-5-(6-((4-((6-((1 R,4R)-bicyclo[2.2.1]hept-5-ene-2-carboxamido)hexyl)carbamoyl)benzyl)amino)-2-chloro-9H-purin-9-yl)-3,4-dihydroxytetrahydrofuran-2-yl)methoxy)(hydroxy)phosphoryl)methyl)phosphonic acid (40) is attached to N-(4-(5,5-difluoro-1,3,7,9-tetramethyl-5H-4λ⁴, 5λ⁴-dipyrrolo[1,2-c:2′,1′-f][1,3,2]diazaborinin-10-yl)butyl)-4-(6-methyl-1,2,4,5-tetrazin-3-yl)benzamide (44) via an inverse electron demand Diels-Alder reaction (IEDDA) to give 45 as a mixture of endo/exo and 1,4- and 2,5 dihydropyridazine isomers

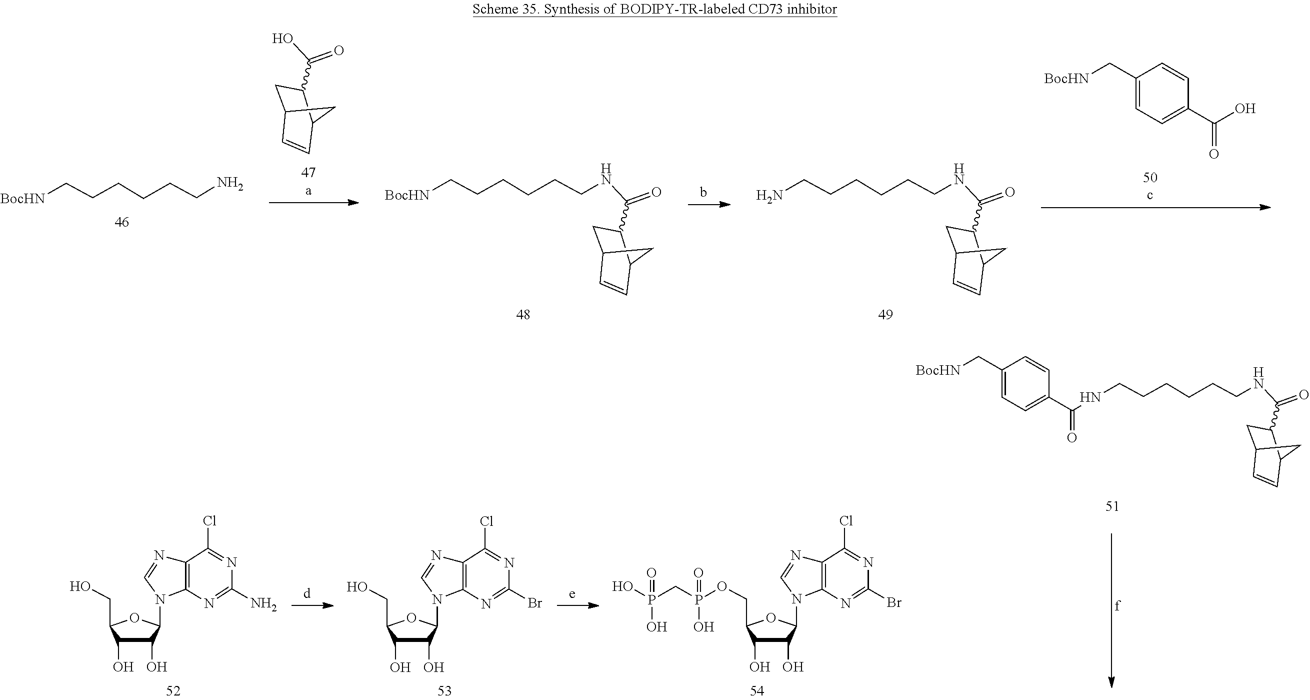

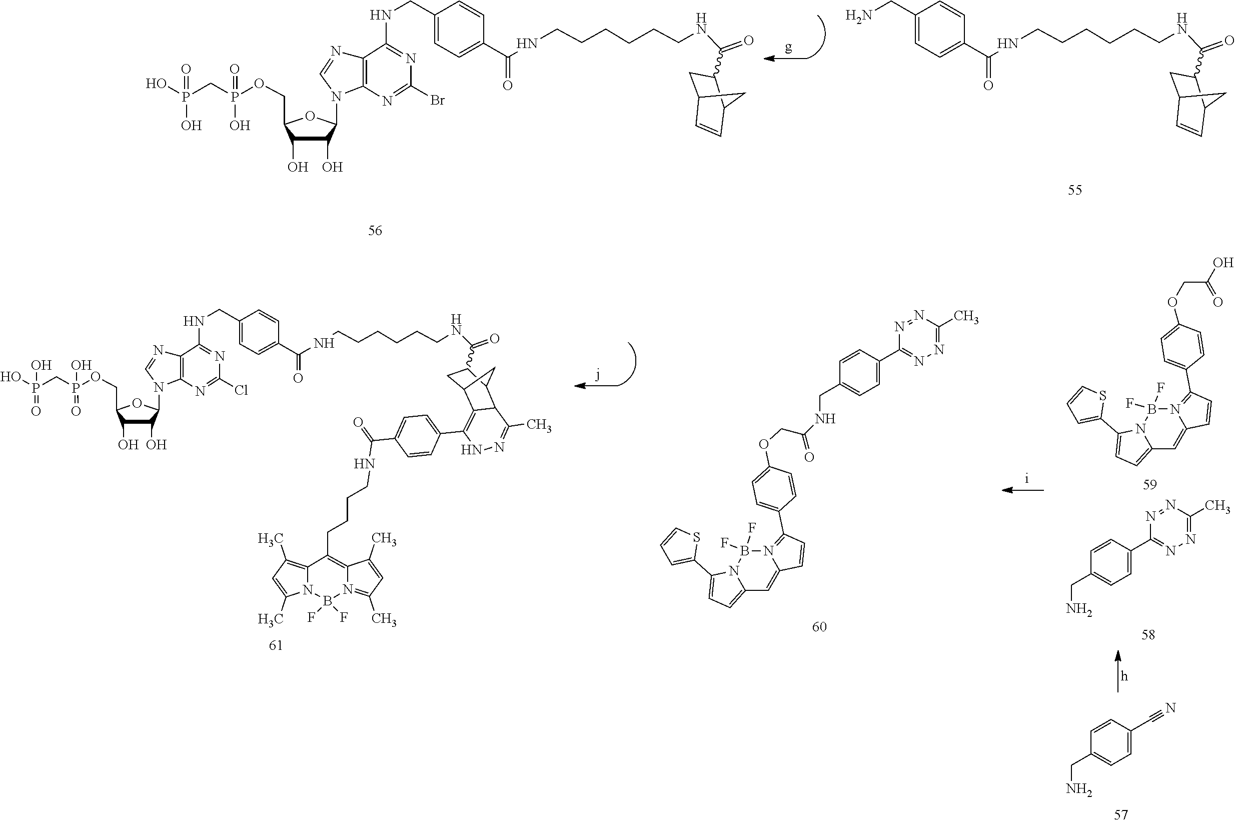

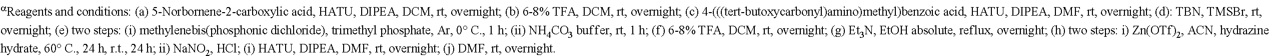

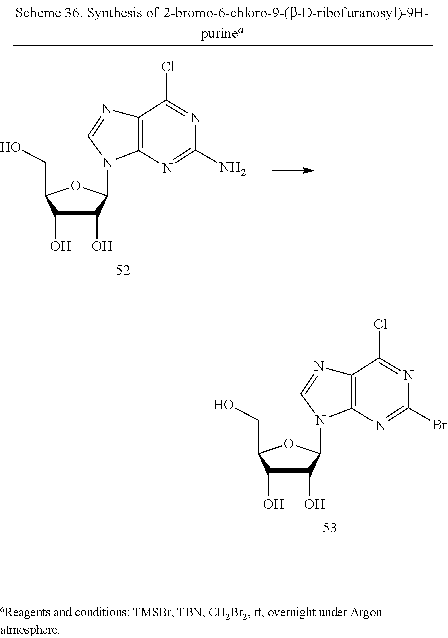

Commercially availabe 2-amino-6-choro-9-(β-D-ribofuranosyl)purine (52) was reacted by a non-aqueous diazotization reaction using tert-butyl nitrite (TBN) and trimethylsilybromide (TMSBr) in dibromomethane. The reaction was carried out according to an altered previously published method to affording 53.^{31, 32}

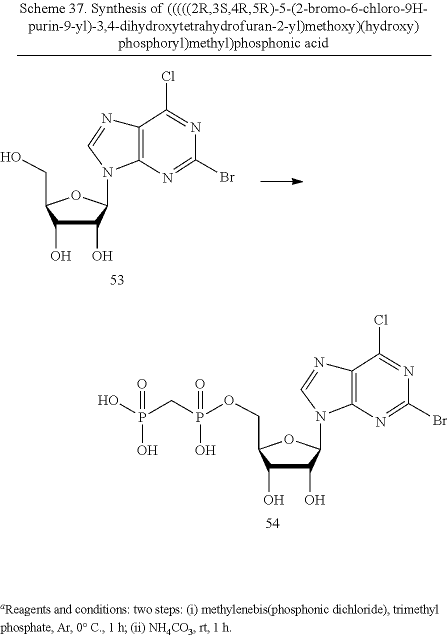

2-bromo-6-chloro-9-(β-D-ribofuranosyl)-9H-purine (53) was phosphonylated using methylenbis(phosphonic dichloride) in trimethyl phosphate, followed by quenching with aqueous TEAC buffer. Both reaction steps were carried out according to an optimized previously published method affording 54.^{13, 18}

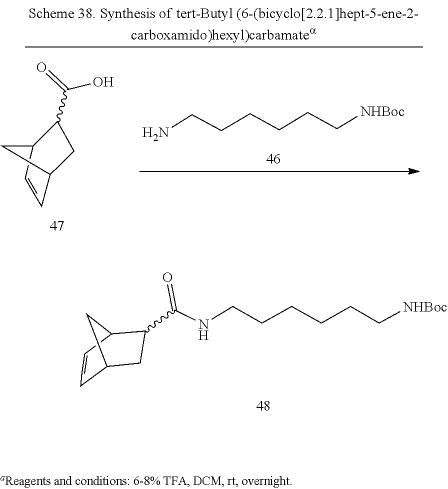 [SMILES: O=C(O)C1CC2CC=CC1C2 ; CC(C)(C)OC(=O)NCCCCCCN ; CC(C)(C)OC(=O)NCCCCCCNC(=O)C1CC2CC=CC1C2]

A mixture of endo- and exo-isomers of 5-norbornene-2-carboxylic acid (47) was attached to commercially available N-boc-1,6-hexanediamine (46) via amide coupling with HATU and DIPEA in DCM to give 48.

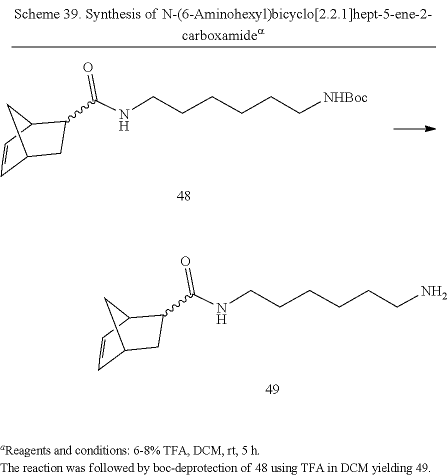 [SMILES: CC(C)(C)OC(=O)NCCCCCCNC(=O)C1CC2CC=CC1C2 ; NCCCCCCNC(=O)C1CC2CC=CC1C2]

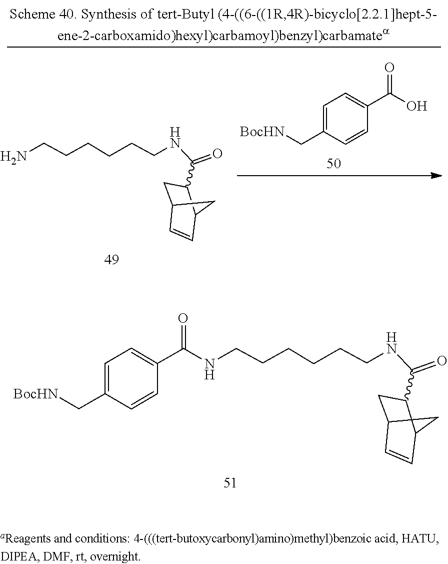 [SMILES: NCCCCCCNC(=O)C1CC2C=CC1C2 ; CC(C)(C)OC(=O)NCc1ccc(C(=O)O)cc1 ; CC(C)(C)OC(=O)NCc1ccc(C(=O)NCCCCCCNC(=O)C2CC3C=CC2C3)cc1]

4-(((tert-butoxycarbonyl)amino)methyl)benzoic acid (50) was attached to 49 via amide coupling with HATU and DIPEA in DMF to give 51.

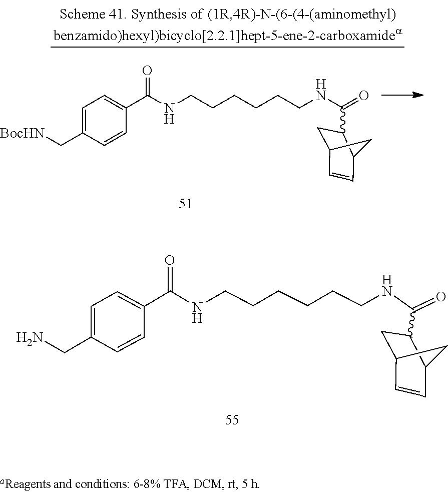 [SMILES: CC(C)(C)OC(=O)NCc1ccc(C(=O)NCCCCCCNC(=O)C2CC3C=CCC2C3)cc1 ; CC1(C(=O)NCCCCCCNC(=O)c2ccc(CN)cc2)CC2C=CCC1C2]

The reaction was followed by boc-deprotection of 51 using TFA in DCM yielding 55.

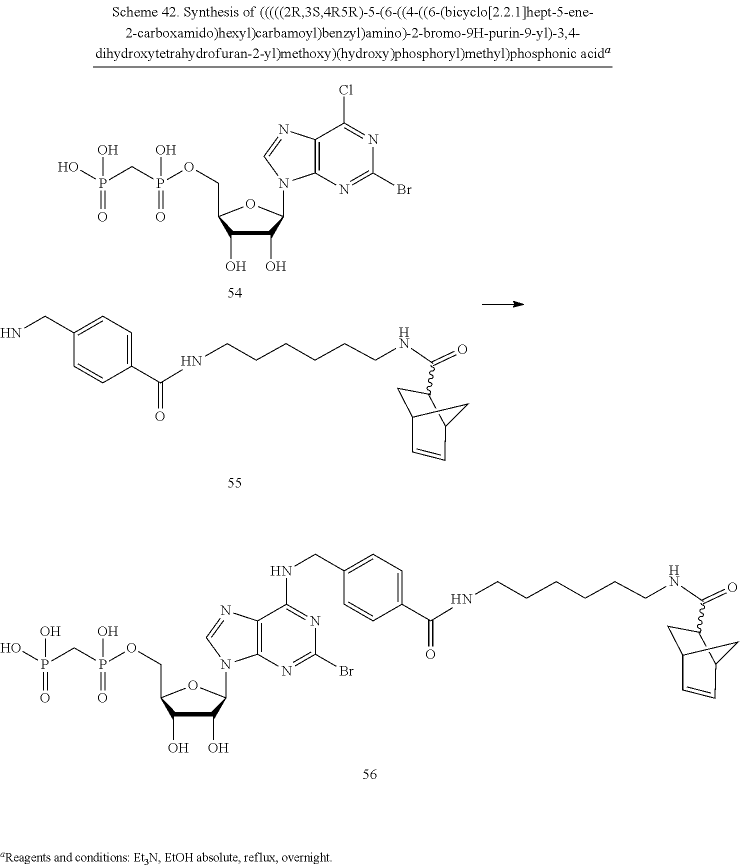

In the reaction step, nucleophilic substitution of 54 by the primary amine 55 in absolute ethanol in the presence of triethyl amine under reflux conditions, followed by RP-HPLC purification, yielded the desired final product 56.¹⁸

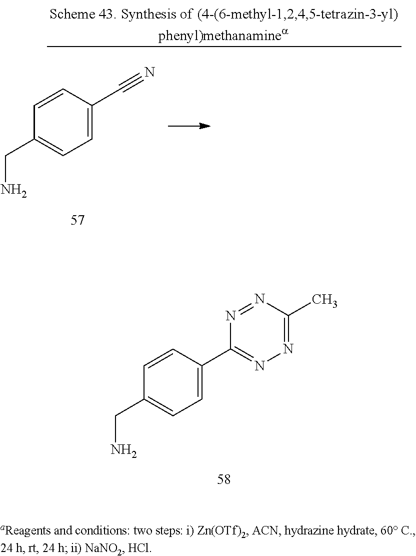

4-(aminomethyl)benzonitrile (57), Zn(OTf)₂ and acetonitrile were added to a solution of hydrazine hydrate 80% w/w, stirred at 60° C. for 24 h and then at rt for another 24 h. Then an aqueous NaNO₂ solution was added slowly followed by conc. HCl until pH 3 is reached and gas evolution is ceased to give 59.³³

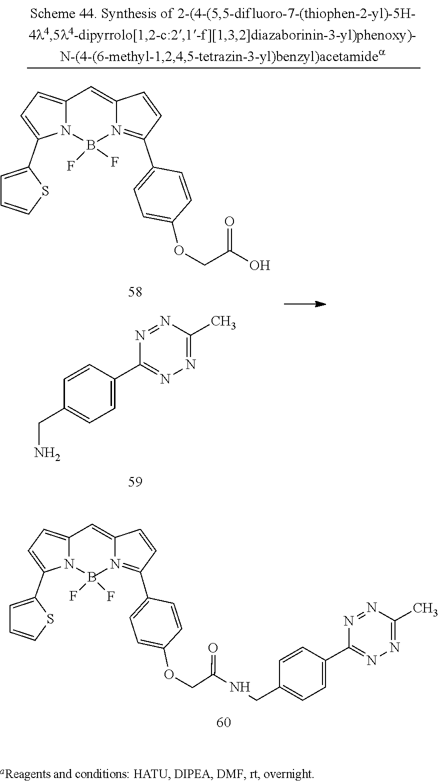

59 was attached to 2-(4-(5,5-difluoro-7-(thiophen-2-yl)-5H-4λ⁴,5λ⁴-dipyrrolo[1,2-c:2′,1′-f][1,3,2]diazaborinin-3-yl)phenoxy)acetic acid (58-BODIPY-TR) via an amide coupling reaction to give 60.

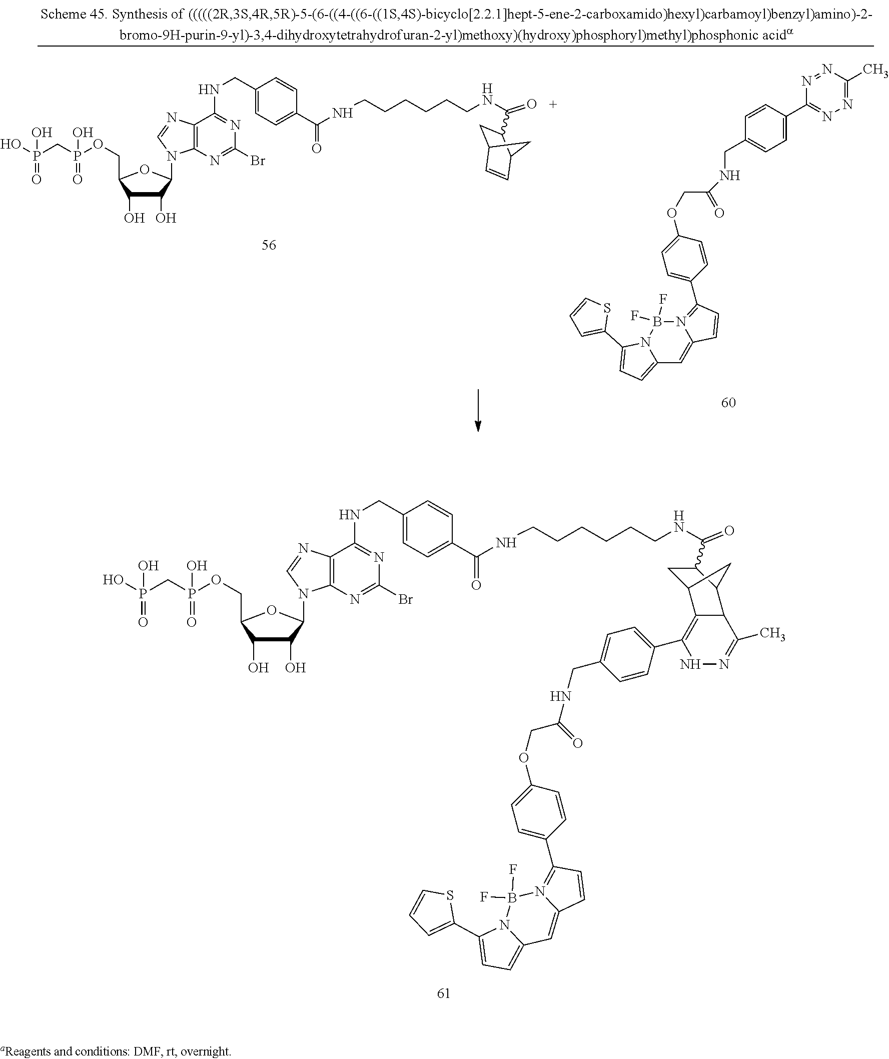

56 is attached to 60 via an inverse electron demand Diels-Alder reaction (IEDDA) to give 61 as a mixture of endo/exo and 1,4- and 2,5-dihydropyridazine isomers.

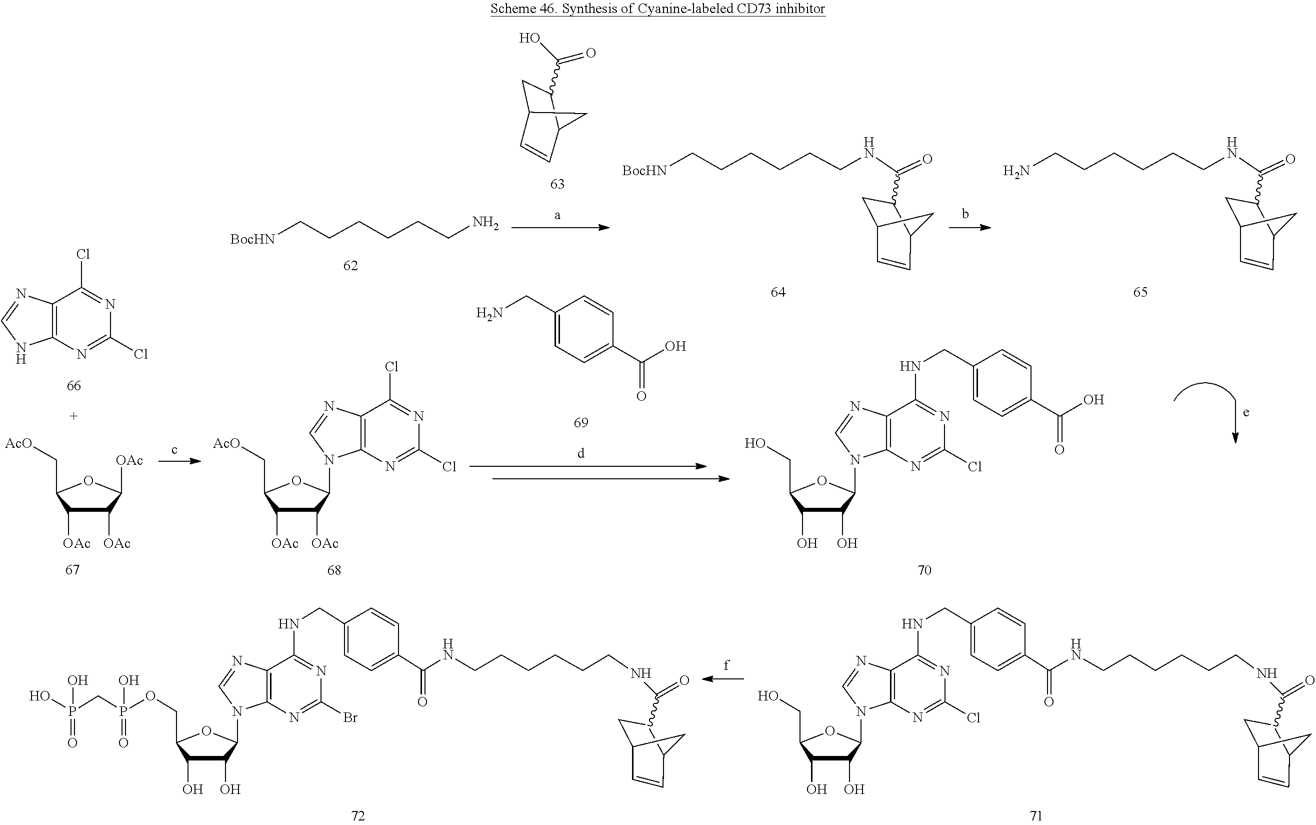

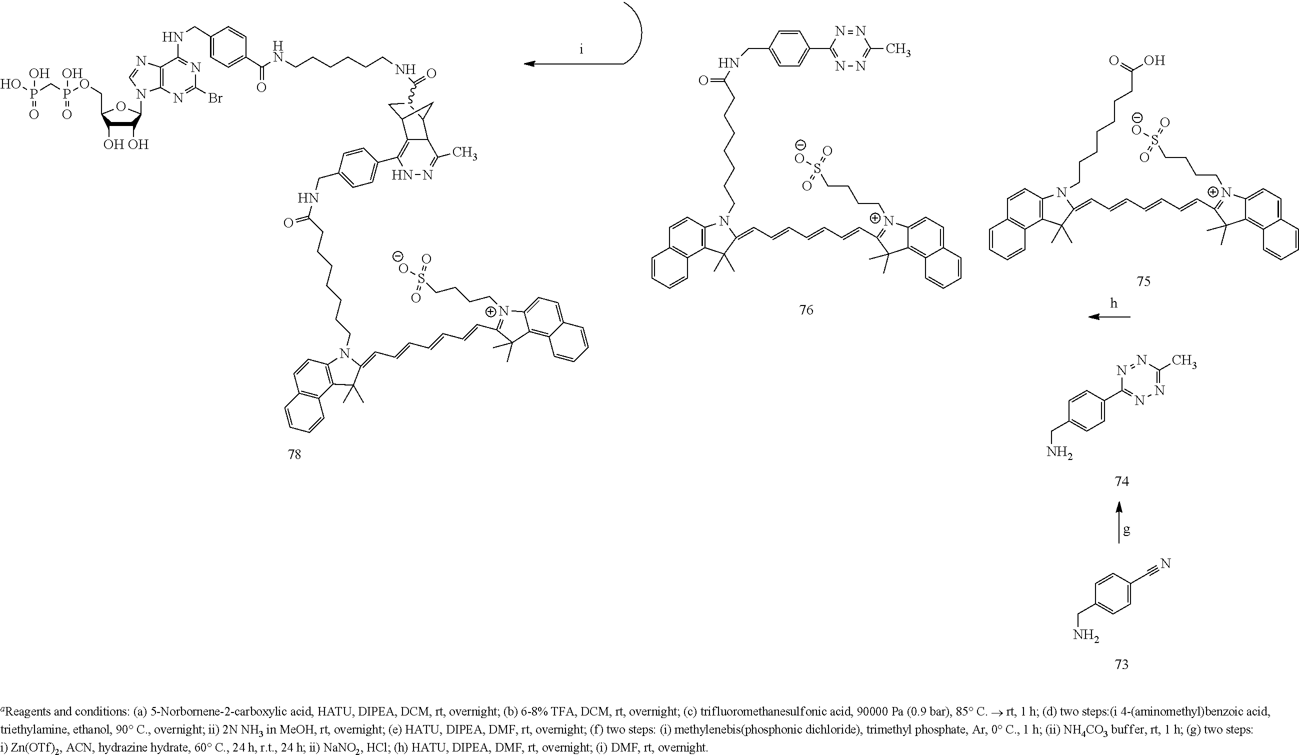

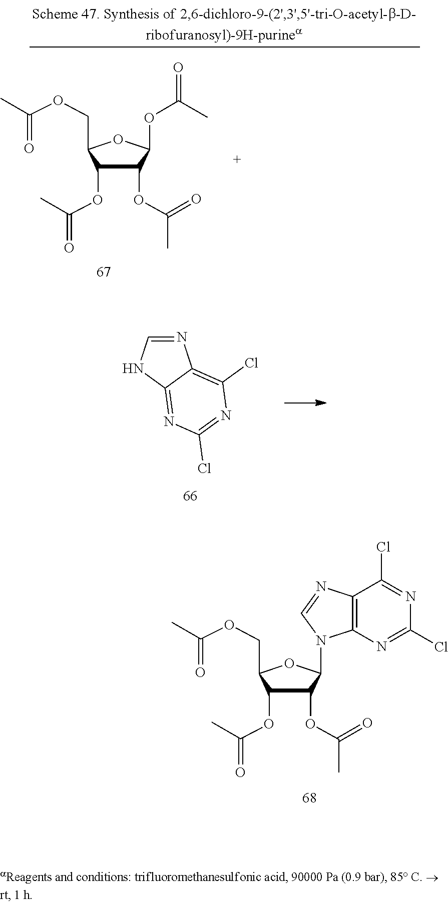

2,6-Dichloro-9-(2′,3′,5′-tri-O-acetyl-β-D-ribofuranosyl)-9H-purine (68) was synthesized starting from 1,2,3,5-tetraacetyl-β-D-ribofuranose (67) and 2,6-dichloropurine (66). The reaction was carried out using previously published reaction conditions.¹³ Both compounds were melted at 85° C. and reacted after the addition of trifluoromethanesulfonic acid.

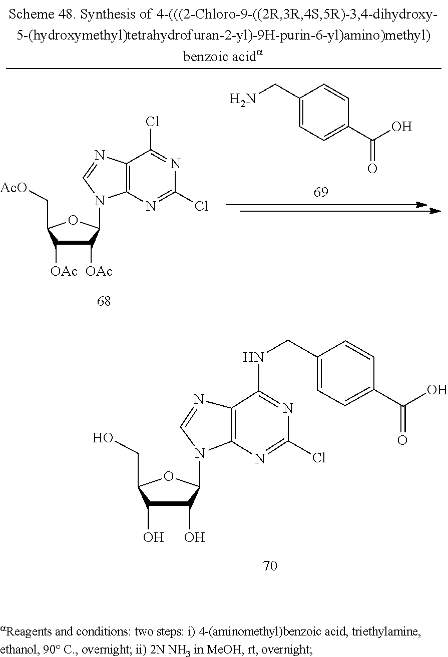

2′,3′,5′-Tri-O-acetyl-2,6-dichloropurine riboside (68) was reacted with 4-(aminomethyl)benzoic acid (69) under basic conditions in the presence of triethylamine. The mixture was then refluxed at 90° C. overnight.¹⁸ After TLC indicated the completion of the reaction the volatiles were evaporated, 2N NH₃ in MeOH was given to the crude product and the mixture was stirred at room temperature overnight. After evaporation of the solvent, ethanol abs. was given to the flask and heated to 100° C. to recrystallized the unreacted 4-(aminomethyl)benzoic acid. After filtration the volatiles were evaporated and 70 could be crystallized from methanol.

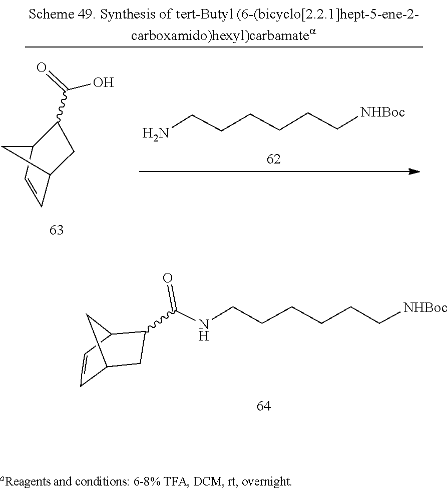 [SMILES: O=C(O)C1CC2CC=CC1C2 ; CC(C)(C)OC(=O)NCCCCCCN ; CC(C)(C)OC(=O)NCCCCCCNC(=O)C1CC2CC=CC1C2]

A mixture of endo- and exo-isomers of 5-norbornene-2-carboxylic acid (63) was attached to commercially available N-boc-1,6-hexanediamine (62) via amide coupling with HATU and DIPEA in DCM to give 64.

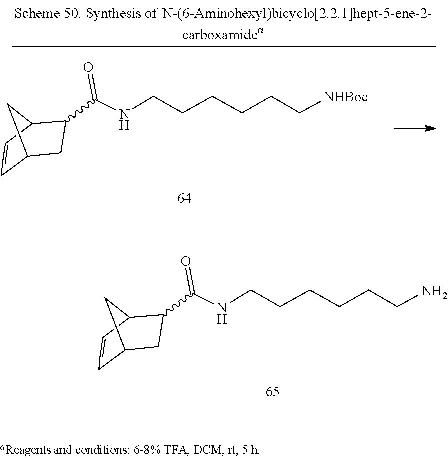 [SMILES: CC(C)(C)OC(=O)NCCCCCCNC(=O)C1CC2CC=CC1C2 ; NCCCCCCNC(=O)C1CC2CC=CC1C2]

The reaction was followed by boc-deprotection of 64 using TFA in DCM yielding 65.

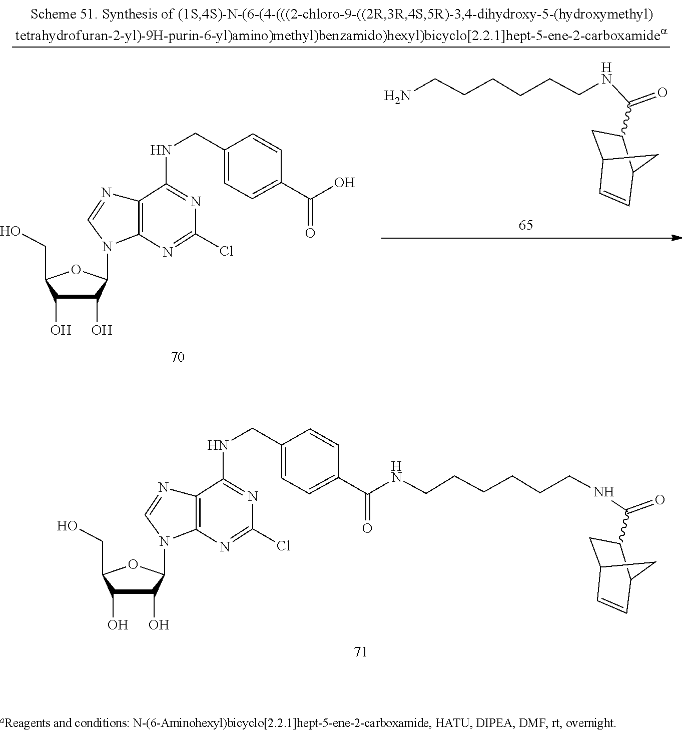

N-(6-Aminohexyl)bicyclo[2.2.1]hept-5-ene-2-carboxamide (65) was attached to 4-(((2-Chloro-9-((2R,3R,4S,5R)-3,4-dihydroxy-5-(hydroxymethyl)tetrahydrofuran-2-yl)-9H-purin-6-yl)amino)methyl)benzoic acid (70) via an amide coupling reaction to give 71.

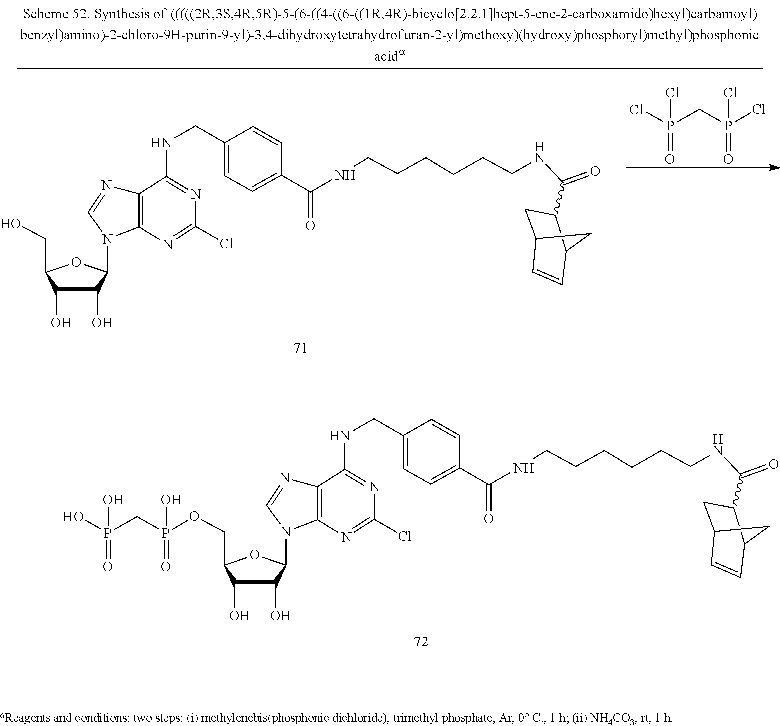

(1S,4S)—N-(6-(4-(((2-chloro-9-((2R,3R,4S,5R)-3,4-dihydroxy-5-(hydroxymethyl)tetrahydrofuran-2-yl)-9H-purin-6-yl)amino)methyl)benzamido)hexyl)bicyclo[2.2.1]hept-5-ene-2-carboxamide (71) was phosphonylated using methylenbis(phosphonic dichloride) in trimethyl phosphate, followed by quenching with aqueous ammonium bicarbonate buffer. Both reaction steps were carried out according to an optimized previously published method affording 72.^{13, 18}

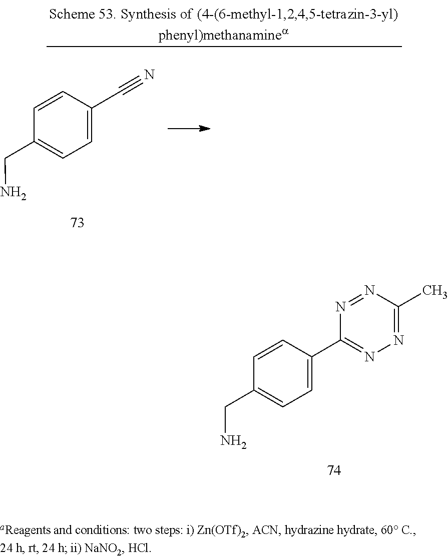

4-(aminomethyl)benzonitrile (73), Zn(OTf)₂ and acetonitrile were added to a solution of hydrazine hydrate 80% w/w, stirred at 60° C. for 24 h and then at rt for another 24 h. Then an aqueous NaNO₂ solution was added slowly followed by conc. HCl until pH 3 is reached and gas evolution is ceased to give 74.³³

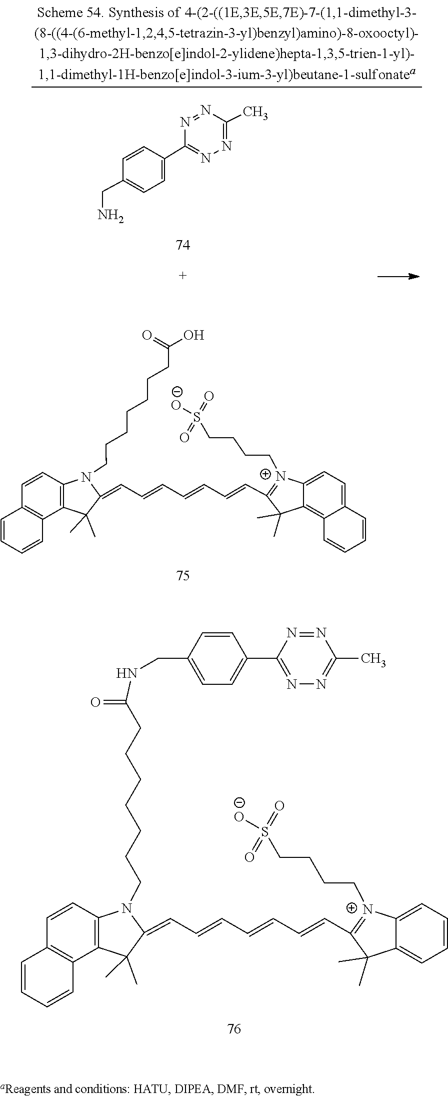 [SMILES: Cc1nnc(-c2ccc(CN)cc2)nn1 ; CC1(C)C(/C=C/C=C/C=C/C=C2/N(CCCCCCCC(=O)O)c3ccc4ccccc4c3C2(C)C)=[N+](CCCCS(=O)(=O)[O-])c2ccc3ccccc3c21 ; Cc1nnc(-c2ccc(CNC(=O)CCCCCCCN3/C(=C/C=C/C=C/C=C/C4=[N+](CCCCS(=O)(=O)[O-])c5ccccc5C4(C)C)C(C)(C)c4c3ccc3ccccc43)cc2)nn1]

74 was attached to 4-(2-((1E,3E,5E,7E)-7-(3-(7-carboxyheptyl)-1,1-dimethyl-1,3-dihydro-2H-benzo[e]indol-2-ylidene)hepta-1,3,5-trien-1-yl)-1,1-dimethyl-1H-benzo[e]indol-3-ium-3-yl)butane-1-sulfonate (75-Cy7.5 (mono SO₃)) via an amide coupling reaction to give 76.

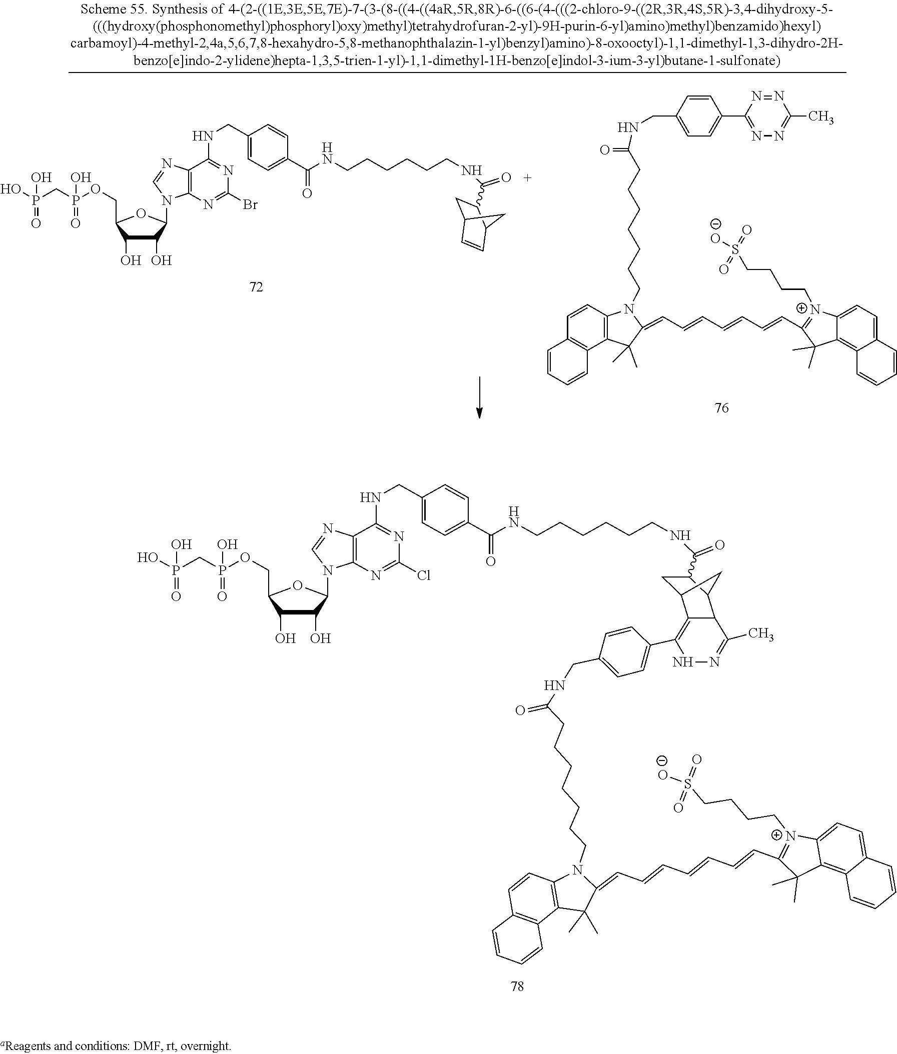

72 is attached to 76 via an inverse electron demand Diels-Alder reaction (IEDDA) to give 78 as a mixture of endo/exo and 1,4- and 2,5 dihydropyridazine isomers.

### REFERENCES

1. Zimmermann, H.; Zebisch, M.; Sträter, N., Cellular function and molecular structure of ecto-nucleotidases. Purinergic Signal 2012, 8 (3), 437-502.
2. Gao, Z. W.; Dong, K.; Zhang, H. Z., The roles of CD73 in cancer. Biomed Res Int 2014, 2014, 460654.
3. Jin, D.; Fan, J.; Wang, L.; Thompson, L. F.; Liu, A.; Daniel, B. J.; Shin, T.; Curiel, T. J.; Zhang, B., CD73 on tumor cells impairs antitumor T-cell responses: a novel mechanism of tumor-induced immune suppression. Cancer Res 2010, 70 (6), 2245-55.
4. Yu, J.; Wang, X.; Lu, Q.; Wang, J.; Li, L.; Liao, X.; Zhu, W.; Lv, L.; Zhi, X.; Yu, J.; Jin, Y.; Zou, Q.; Ou, Z.; Liu, X.; Zhou, P., Extracellular 5′-nucleotidase (CD73) promotes human breast cancer cells growth through AKT/GSK-3β/β-catenin/cyclinD1 signaling pathway. International journal of cancer. Journal international du cancer 2018, 142 (5), 959-967.
5. Bellingan, G.; Maksimow, M.; Howell, D. C.; Stotz, M.; Beale, R.; Beatty, M.; Walsh, T.; Binning, A.; Davidson, A.; Kuper, M.; Shah, S.; Cooper, J.; Waris, M.; Yegutkin, G. G.; Jalkanen, J.; Salmi, M.; Piippo, I.; Jalkanen, M.; Montgomery, H.; Jakanen, S., The effect of intravenous interferon-beta-1a (FP-1201) on lung CD73 expression and on acute respiratory distress syndrome mortality: an open-label study. Lancet Respir Med 2014, 2 (2), 98-107.
6. Spychala, J.; Lazarowski, E.; Ostapkowicz, A.; Ayscue, L. H.; Jin, A.; Mitchell, B. S., Role of Estrogen Receptor in the Regulation of Ecto-5′-Nucleotidase and Adenosine in Breast Cancer. Clinical Cancer Research 2004, 10 (2), 708.
7. Zhou, X.; Zhi, X.; Zhou, P.; Chen, S.; Zhao, F.; Shao, Z.; Ou, Z.; Yin, L., Effects of ecto-5′-nucleotidase on human breast cancer cell growth in vitro and in vivo. Oncology reports 2007, 17 (6), 1341-6.
8. Buisseret, L.; Pommey, S.; Allard, B.; Garaud, S.; Bergeron, M.; Cousineau, I.; Ameye, L.; Bareche, Y.; Paesmans, M.; Crown, J. P. A.; Di Leo, A.; Loi, S.; Piccart-Gebhart, M.; Willard-Gallo, K.; Sotiriou, C.; Stagg, J., Clinical significance of CD73 in triple-negative breast cancer: multiplex analysis of a phase III clinical trial. Ann Oncol 2018, 29 (4), 1056-1062.
9. Augusto, E.; Matos, M.; Sévigny, J.; El-Tayeb, A.; Bynoe, M. S.; Müller, C. E.; Cunha, R. A.; Chen, J.-F., Ecto-5′-Nucleotidase (CD73)-Mediated Formation of Adenosine Is Critical for the Striatal Adenosine A&lt;sub&gt;2A&lt;/sub&gt; Receptor Functions. The Journal of Neuroscience 2013, 33 (28), 11390.
10. Allard, B.; Turcotte, M.; Stagg, J., CD73-generated adenosine: orchestrating the tumor-stroma interplay to promote cancer growth. J Biomed Biotechnol 2012, 2012, 485156.
11. Vijayan, D.; Young, A.; Teng, M. W. L.; Smyth, M. J., Targeting immunosuppressive adenosine in cancer. Nature Reviews Cancer 2017, 17 (12), 709-724.
12. Antonioli, L.; Novitskiy, S. V.; Sachsenmeier, K. F.; Fornai, M.; Blandizzi, C.; Haskó, G., Switching off CD73: a way to boost the activity of conventional and targeted antineoplastic therapies. Drug Discovery Today 2017.
13. Bhattarai, S.; Pippel, J.; Meyer, A.; Freundlieb, M.; Schmies, C.; Abdelrahman, A.; Fiene, A.; Lee, S.-Y.; Zimmermann, H.; EI-Tayeb, A.; Yegutkin, G. G.; Sträter, N.; Müller, C. E. X-Ray Co-Crystal Structure Guides the Way to Subnanomolar Competitive Ecto-5′-Nucleotidase (CD73) Inhibitors for Cancer Immunotherapy. Adv. Therap. 2019, 2, 1900075.
14. Bhattarai, S.; Freundlieb, M.; Pippel, J.; Meyer, A.; Abdelrahman, A.; Fiene, A.; Lee, S.-Y.; Zimmermann, H.; Yegutkin, G. G.; Sträter, N.; EI-Tayeb, A.; Müller, C. E. α,β-Methylene-ADP (AOPCP) derivatives and analogues: development of potent and selective ecto-5′-nucleotidase (CD73) inhibitors. J. Med. Chem. 2015, 58, 6248-6263.
15. Bhattarai, S.; Pippel, J.; Scaletti, E.; Idris, R.; Freundlieb, M.; Rolshoven, G.; Renn, C.; Lee, S.-Y.; Abdelrahman, A.; Zimmermann, H.; El-Tayeb, A.; Müller, C. E.; Sträter, N. 2-substituted α,β-methylene-ADP derivatives: potent competitive ecto-5′-nucleotidase (CD73) inhibitors with variable binding modes. J. Med. Chem. 2020, 63, 2941-2957.
16. Junker, A.; Renn, C.; Dobelmann, C.; Namasivayam, V.; Jain, S.; Losenkova, K.; Irjala, H.; Duca, S.; Balasubramanian, R.; Chakraborty, S.; Börgel, F.; Zimmermann, H.; Yegutkin, G. G.; Müller, C. E.; Jacobson, K. A. Structure-activity relationship of purine and pyrimidine nucleotides as ecto-5′-nucleotidase (CD73) inhibitors. J. Med. Chem. 2019, 62, 3677-3695.
17. Schäkel, L.; Schmies, C. C.; Idris, R. M.; Luo, X.; Lee, S.-Y.; Lopez, V.; Mirza, S.; Vu, T. H.; Pelletier, J.; Sévigny, J.; Namasivayam, V.; Müller, C. E. Nucleotide Analog ARL67156 as a Lead Structure for the Development of CD39 and Dual CD39/CD73 Ectonucleotidase Inhibitors. Front. Pharmacol. 2020, 11, 1294.
18. Schmies, C. C.; Rolshoven, G.; Idris, R. M.; Losenkova, K.; Renn, C.; Schäkel, L.; Al-Hroub, H.; Wang, Y.; Garofano, F.; Schmidt-Wolf, I. G. H.; Zimmermann, H.; Yegutkin, G. G.; Müller, C. E. Fluorescent Probes for Ecto-5′-nucleotidase (CD73). ACS Med. Chem. Left. 2020, 11, 2253-2260.
19. Vrettos, E. I.; Sayyad, N.; Mavrogiannaki, E. M.; Stylos, E.; Kostagianni, A. D.; Papas, S.; Mavromoustakos, T.; Theodorou, V.; Tzakos, A. G. Unveiling and tackling guanidinium peptide coupling reagent side reactions towards the development of peptide-drug conjugates. RSC Adv. 2017, 7, 50519-50526.
20. Freundlieb, M.; Zimmermann, H.; Müller, C. E. A new, sensitive ecto-5′-nucleotidase assay for compound screening. Analytical Biochemistry 2014, 446, 53-58.
21. Servos, J.; Reilnder, H.; Zimmermann, H. Catalytically active soluble ecto-5?-nucleotidase purified after heterologous expression as a tool for drug screening. Drug Dev. Res. 1998, 45, 269-276.
22. Stagg, J.; Divisekera, U.; McLaughlin, N.; Sharkey, J.; Pommey, S.; Denoyer, D.; Dwyer, K. M.; Smyth, M. J. Anti-CD73 antibody therapy inhibits breast tumor growth and metastasis. Proceedings of the National Academy of Sciences of the United States of America 2010, 107, 1547-1552.
23. Yung-Chi, C.; Prusoff, W. H. Relationship between the inhibition constant (KI) and the concentration of inhibitor which causes 50 percent inhibition (150) of an enzymatic reaction. Biochem. Pharmacol. 1973, 22, 3099-3108.
24. Schäkel, L.; Schmies, C. C.; Idris, R. M.; Luo, X.; Lee, S.-Y.; Lopez, V.; Mirza, S.; Vu, T. H.; Pelletier, J.; Sévigny, J.; Namasivayam, V.; Müller, C. E. Nucleotide Analog ARL67156 as a Lead Structure for the Development of CD39 and Dual CD39/CD73 Ectonucleotidase Inhibitors. Front. Pharmacol. 2020, 11, 1294.
25. Rafehi, M.; Malik, E. M.; Neumann, A.; Abdelrahman, A.; Hanck, T.; Namasivayam, V.; Müller, C. E.; Baqi, Y. Development of Potent and Selective Antagonists for the UTP-Activated P2Y4 Receptor. J. Med. Chem. 2017, 60, 3020-3038.
26. Rafehi, M.; Burbiel, J. C.; Attah, I. Y.; Abdelrahman, A.; Müller, C. E. Synthesis, characterization, and in vitro evaluation of the selective P2Y2 receptor antagonist AR-C118925. Purinergic Signal. 2017, 13, 89-103.
27. Attah, I. Y.; Neumann, A.; Al-Hroub, H.; Rafehi, M.; Baqi, Y.; Namasivayam, V.; Müller, C. E. Ligand binding and activation of UTP-activated G protein-coupled P2Y2 and P2Y4 receptors elucidated by mutagenesis, pharmacological and computational studies. Biochim. Biophys. Ada Gen. Subj. 2020, 1864, 129501.
28. Prante, O.; Hocke, C.; Loeber, S.; Huebner, H.; Gmeiner, P.; Kuwert, T., Tissue distribution of radioiodinated FAUC113: assessment of a pyrazolo(1,5-a) pyridine based dopamine D4 receptor radioligand candidate. Nuklearmedizin. Nuclear medicine 2006, 45 1, 41-8.
29. Zhang F. et al., Compilation of 222 drugs' plasma protein binding data and guidance for study designs. Drug Discovery Today 2012, 17 (9-10), 475-485.
30. Tiller P. R., Immobilized human serum atbumin: liquid chromatography/mass spectrometry as a method of determining drug-protein binding. Rapid Commun Mass Spectrom 1995; 9(4):261-3.
31. Francom, R; Janeba, Z.; Shibuya, S.; Robins, M. J. Nucleic acid related compounds. 116. Nonaqueous diazotization of aminopurine nucleosides. Mechanistic considerations and efficient procedures with tert-butyl nitrite or sodium nitrite. J. Org. Chem. 2002, 67, 6788-6796.
32. Francom, R; Robins, M. J. Nucleic acid related compounds. 118. Nonaqueous diazotization of aminopurine derivatives. Convenient access to 6-halo- and 2,6-dihalopurine nucleosides and 2′-deoxynucleosides with acyl or silyl halides. J. Org. Chem. 2003, 68, 666-669.
33. Yang, J.; Karver, M. R.; Li, W.; Sahu, S.; Devaraj, N. K. Metal-catalyzed one-pot synthesis of tetrazines directly from aliphatic nitriles and hydrazine. Angew. Chem. Int. Ed. Engl. 2012, 51, 5222-5225.

1. A compound according to general formula (I) 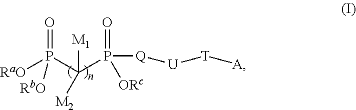
wherein
R^{a}, R^{b} and RC are independently selected from the group consisting of H, —(C₁-C₆)alkyl, —(C₆-C₁₀)aryl, —C(O)(C₁-C₆)alkyl, —(C₁-C₆)alkyl(C₆-C₁₀)heteroaryl, —(C₆-C₁₀)heteroaryl and —C(O)aryl;
M₁ and M₂ are independently selected from the group consisting of H, —OH and halogen;
n is 1 to 6, preferably 1 to 3, more preferably 1;
Q is selected from the group consisting of O, S, CH₂, NH, preferably O;
U is selected from the group consisting of O, S, CH₂, (CH₂)₂, NH, preferably CH₂;
T is selected from the group consisting of 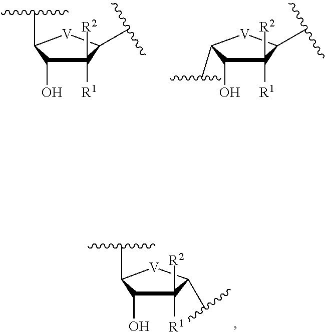
preferably 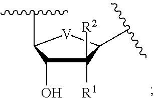
V is selected from the group consisting of O, NH, S, CH₂; preferably O
R¹ and R² are independently selected from the group consisting of H, OH, SH, —O(C₁-C₆)alkyl, —S(C₁-C₆)alkyl, —NH₂, —NH(C₁-C₆)alkyl, —N₃ and halogen, preferably H and —OH;
A is selected from the group consisting of 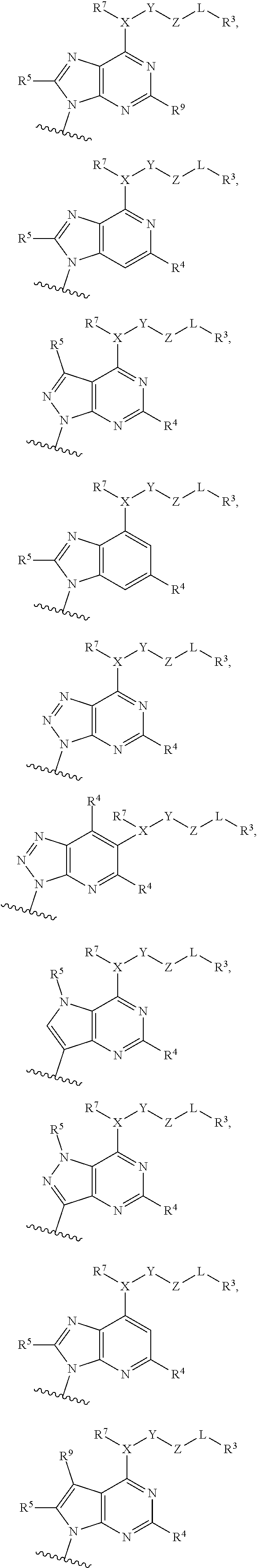
preferably 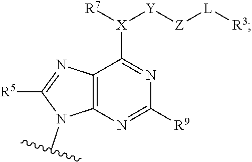
X is selected from the group consisting of O, S, N, 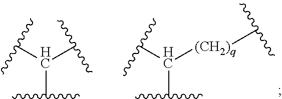
preferably N
q in 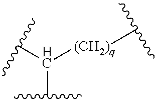
is 0 to 6, preferably 0 to 4, more preferably 0
Y is selected from the group consisting of —(C₆-C₁₀)aryl-, —(CH₂)ₚ—, —(CH₂)ₚC(O)—, —(C₆-C₁₀)heteroaryl(CH₂)ₚ—, —(C₆-C₁₀)heteroaryl-, —(CH₂)ₚ(C₆-C₁₀)aryl-, and —(C₆-C₁₀)arylC(O)—, preferably —(CH₂)ₚ—, or —(C₆-C₁₀)arylC(O)—;
Z is selected from the group consisting of 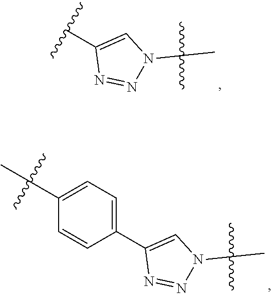 [SMILES: *c1cn(*)nn1 ; *c1ccc(-c2cn(*)nn2)cc1]
NH, O, S, 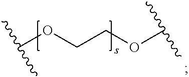
CH₂)ₚ—, —(C₆-C₁₀)aryl —(C₆-C₁₀)aryl(CH₂)ₚ, —C(O)(CH₂)ₚ—, —(C₆-C₁₀)heteroaryl(CH₂)ₚ—, —(C₆-C₁₀)heteroaryl-, —(C₆-C₁₀)arylC(O)—, preferably 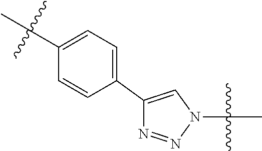 [SMILES: *c1ccc(-c2cn(*)nn2)cc1]
and —(C₆-C₁₀)arylC(O)—, wherein optionally the aryl groups may be substituted by one or more substituents selected from the group consisting of —(C₁-C₆)alkyl, —(C₂-C₆)alkynyl, -halogen, -trifluoromethyl, —OH, —SH, —NH₂, —SO₂NH₂, —(C₁-C₆)alkylOH, —O(C₁-C₆)alkyl, —SO₃H, —(CH₂)₁₋₆COOH, —COOH, —C(O)NH₂, —SO₃(C₁-C₆)alkyl, (C₅-C₆)arylCH₂C(O)—, —C(O)NH(CH₂)ₒNH₂;
o is an integer from 1 to 8, preferably 1 to 4, most preferably 4;
p is an integer from 1 to 6, preferably 1 to 4, most preferably 4;
L comprises L¹ and L², with L¹ connected to L², L¹-L² wherein L¹ is selected from the group consisting of absent, —(CH₂)_{q}—, —C(O)NH(CH₂)ₚNH—, —(CH₂)_{q}(C₅-C₁₀)aryl-, —(C₁-C₁₀)alkynyl-, —(C₆-C₁₀)aryl(CH₂)ₚ—, 1-halo-1-vinyl, —(C₆-C₁₀)heteroaryl(CH₂)ₚ—, —(C₆-C₁₀)heteroaryl-, and —(C₆-C₁₀)arylC(O)—, 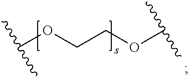
s is an integer from 1 to 60, preferably 1 to 50, more preferably 2 to 30;
q is an integer from 1 to 10, preferably 1 to 6, most preferably 5; and
L² is selected from the group consisting of absent, 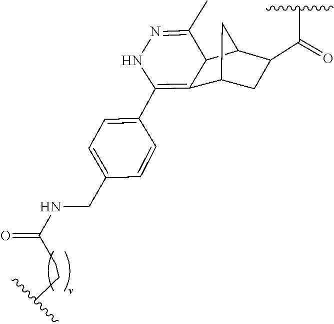
v, is 1 to 9, preferably 1 to 7;
R³ is selected from the group consisting of
i), 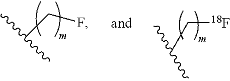
m is an integer of from 2 to 10; or 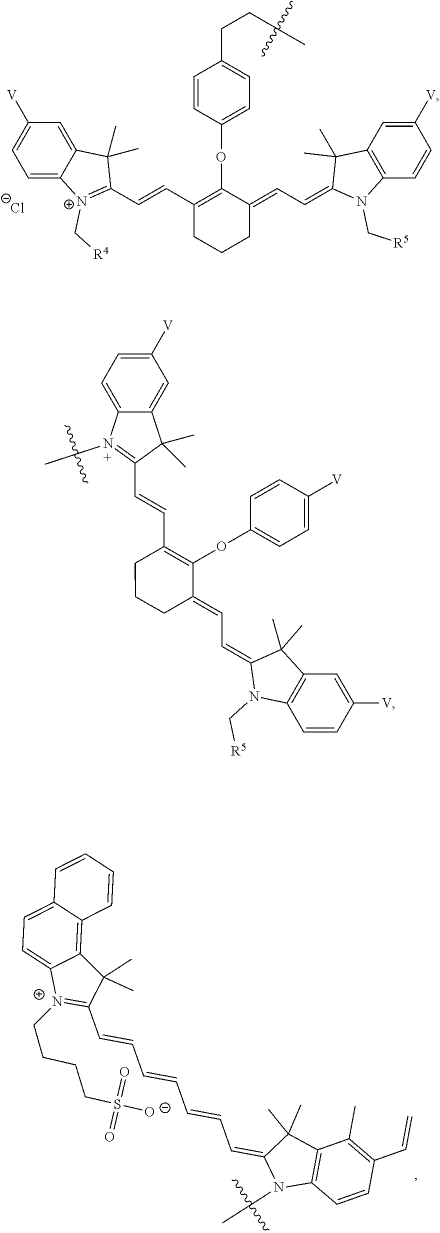 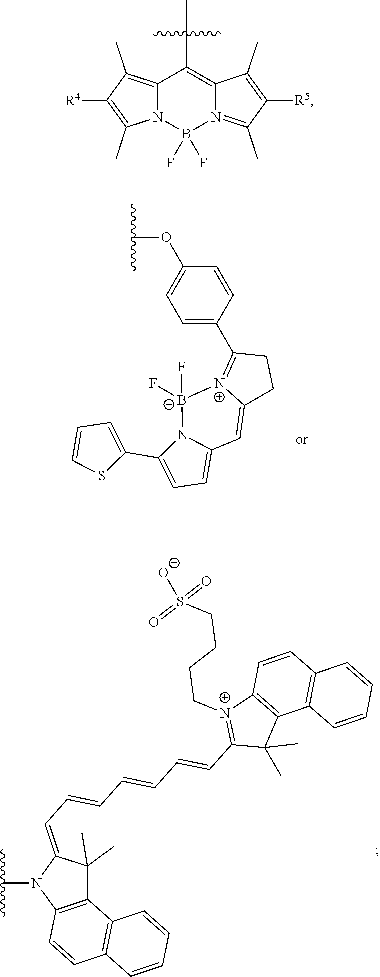
iii) a fluorophore moiety selected from the group consisting of FITC, Fluorescein, NBD, Dansyl, Squaraine Rotaxane, Bodipy FL, Bodipy TR, Bodipy 630/650 X, Bodipy 650/655 X, Texas Red, y5, 1-pyrene, EVOBlue 30, Alexa Fluor 532, Alexa Fluor 488-5, 488-6, or mixture thereof, Tamra, Tamra 5/6-X-SE, Alexa Fluor 488 azide 5 isomer, Alexa Fluor 488 5 isomer, Alexa Fluor 488 5/6 mixed isomers, NIR dye 700, NIR dye 800, Janelia Fluor 549 amide, Janelia Fluor 646 amide, and derivates, analogs and related fluorophores thereof; or
iv) a chelating moiety binding a radioactive metal,
wherein the chelating moiety is selected from the group consisting of 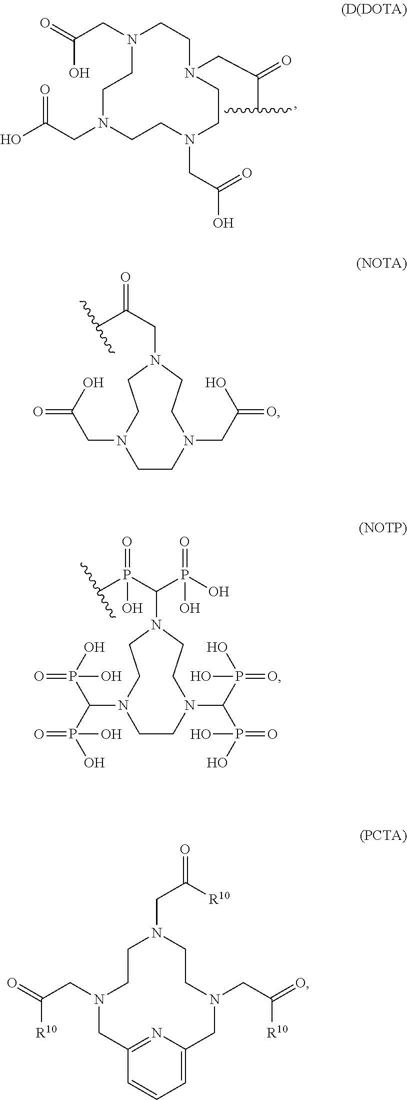 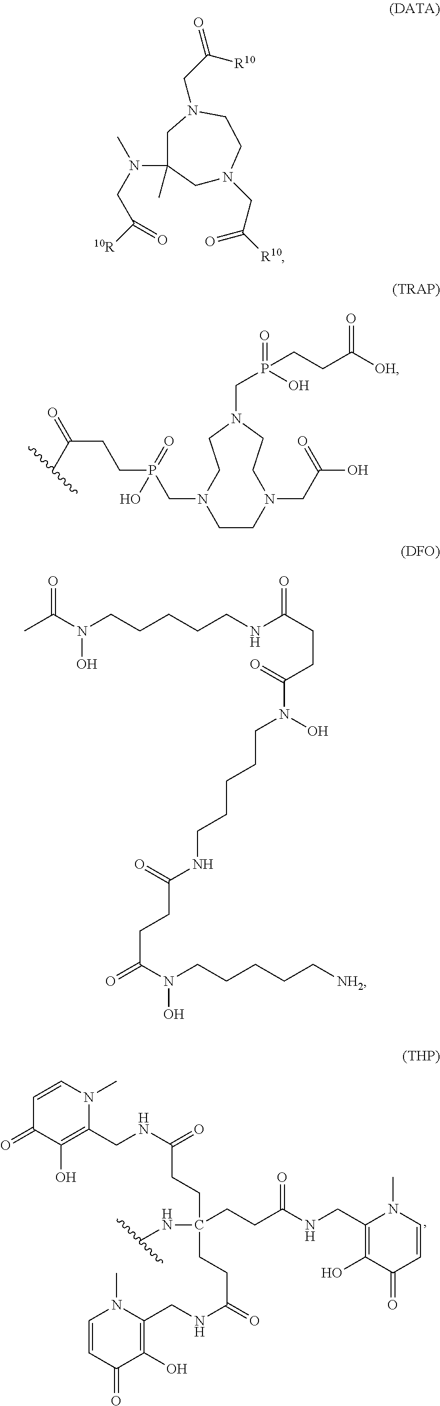 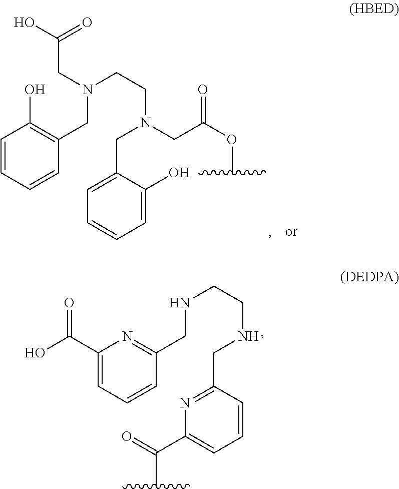 [SMILES: *OC(=O)CN(CCN(CC(=O)O)Cc1ccccc1O)Cc1ccccc1O ; *C(=O)c1cccc(CNCCNCc2cccc(C(=O)O)n2)n1]
and
the radioactive metal is selected from the group consisting of ⁶⁴Cu, ⁶⁸Ga, ¹⁷⁷Lu, ⁹⁰Y, ⁸⁹Zr, ²¹¹At, ²¹²Pb, ¹⁸⁸Rh, ¹⁶⁶Ho, ²²⁵Ac, ^{99m}Tc or ¹¹¹In, ¹²³I, ¹³¹I;
R⁴, R⁵ are independently selected from the group consisting of H, halogen, —(C₁-C₆)alkyl, —(C₁-C₆)aryl, —NH₂, —N₃, —(C₁-C₆)alkynyl, —(C₆-C₁₀)aryl(C₁-C₆)alkyl, -1-halogen-1-vinyl, (C₆-C₁₀)heteroaryl(C₁-C₆)alkyl-, —(C₆-C₁₀)heteroaryl, —C(O)(C₆-C₁₀)aryl-, —OR⁶, —SR⁶, —NHR⁶, —NR⁶R⁷, —SiR⁶R⁷R⁸, —OC(O)R⁶, —C(O)R⁶, —COOR⁶, —CONR⁶R⁷, —OC(O)NR⁶R⁷, —NR⁶C(O)R⁷, —NR⁶COOR⁷, —NHC(NH₂)═NR⁶, —S(O)R⁶, —SO₂NR⁶R⁷, —NR⁶SO₂R⁷, —CN, and —NO₂; or
R⁶, R⁷, R⁸ are independently selected from the group consisting of H, halogen, —(C₁-C₆)alkyl, (C₁-C₆)aryl, —NH₂, —N₃, —(C₁-C₆)alkynyl, —(C₆-C₁₀)aryl(C₁-C₆)alkyl, -1-halogen-1-vinyl, —(C₁-C₆)alkyl(C₆-C₁₀)heteroaryl, —(C₆-C₁₀)heteroaryl, —C(O)(C₆-C₁₀)aryl-, —SO₃H, —OH, and —SH;
R⁹ is selected from the group consisting of halogen, (C₁-C₆)alkyl, —(C₁-C₆)aryl, —NH₂, —N₃, —(C₁-C₆)alkynyl, —(C₆-C₁₀)aryl(C₁-C₆)alkylenyl, 1-halogen-1-vinyl, —(C₆-C₁₀)heteroaryl(C₁-C₆)alkyl-, —(C₆-C₁₀)heteroaryl, —C(O)(C₆-C₁₀)aryl-, —OR⁶, —SR⁶, —NHR⁶, —NR⁶R⁷, —SiR⁶R⁷R⁸, —OC(O)R⁶, —C(O)R⁶, —COOR⁶, —CONR⁶R⁷, —OC(O)NR⁶R⁷, —NR⁶C(O)R⁷, —NR⁶COOR⁷, —NHC(NH₂)═NR⁶, —S(O)R⁶, —SO₂NR⁶R⁷, —NR⁶SO₂R⁷, —CN, and —NO₂;
wherein aryl or heteroaryl of R⁴, R⁵, R⁶, R⁷, R⁸, and R⁹ is optionally substituted with one or more substituents selected from the group consisting of —(C₁-C₆)alkyl, halogen, -trifluoromethyl, —OH, —SH, —NH₂, —SO₂NH₂, —(C₁-C₆)alkylhydroxy, —(C₁-C₆)alkoxy, —SO₃H, —COO(C₁-C₆)alkyl, —SO₃(C₁-C₆)alkyl, —C(O)(C₅-C₆)aryl;
R¹⁰ is OH or 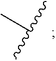
with the provisio that
if R⁷ is H, A is 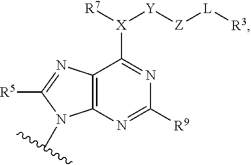
R⁵ is H, X is N, Y is CH₂, Z is phenyl, L is —C(O)NH(CH₂)₁₋₄ NH—, and R⁹ is H or Cl, then R₃ is not fluorescein;
or a pharmaceutically acceptable salt thereof.

2. The compound of claim 1, wherein
R^{a}, R^{b} and R^{c} are H; and/or
M₁ and M₂ are H; and/or
n is 1; and/or
Q is O; and/or
U is CH₂; and/or
R¹ is —OH; and/or
R² is H; and/or
A is 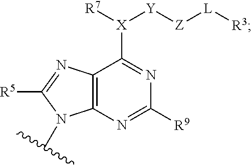
and/or
R⁴ is H, —(C₁-C₆)alkyl or halogen, preferably H or halogen, more preferably halogen; and/or
R⁵ is H and/or
R⁶ is H, —(C₁-C₆)alkyl, —(C₆-C₁₀)aryl(C₁-C₆)alkyl, —(C₁-C₆)alkyl(C₆-C₁₀)heteroaryl; and/or
R⁷ is H or —(C₁-C₆)alkyl, preferably H or (C₂-C₄)alkyl; more preferably (C₃)alkyl and/or
R⁸ is H or —(C₁-C₆)alkyl; and/or
R⁹ is H, halogen, or —(C₁-C₆)alkyl, preferably H or halogen; and/or
X is N; and/or
Y is —(CH₂)ₚ— and/or
Z is 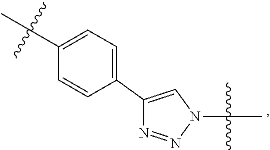 [SMILES: *c1ccc(-c2cn(*)nn2)cc1]
—(C₆-C₁₀)arylC(O);
and/or
L¹ is absent, or —C(O)NH(CH₂)₁₋₄NH—; and/or
L² is selected from the group consisting of absent, and 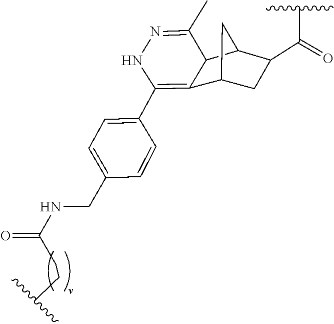
v, is 1 to 9, preferably 1 to 7; 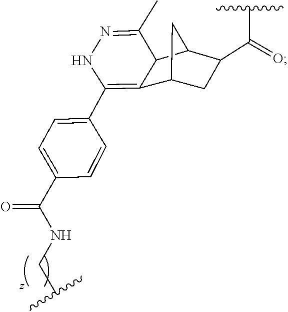
z is 1 to 6, preferably 1 to 5, more preferably 5; and/or
R³ is 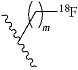
or Fluorescein.

3. The compound of claim 1, wherein
V is O;
Q is O;
U is CH₂; A is 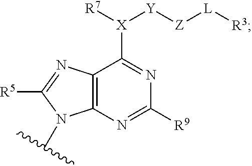
X is N;
Y is CH₂;
Z is phenyl or 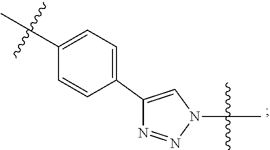 [SMILES: *c1ccc(-c2cn(*)nn2)cc1]
L¹ is absent or —C(O)NH(CH₂)ₚNH—;
p in —C(O)NH(CH₂)ₚNH— is 1 to 6, preferably 4 to 6, most preferably 6;
L² is selected from the group consisting of absent, 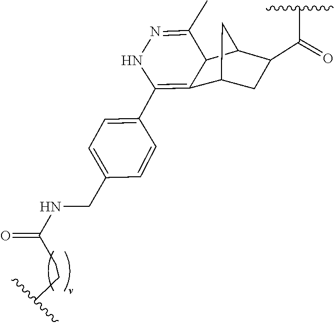
and
v, is 1 to 9, preferably 1 to 7; 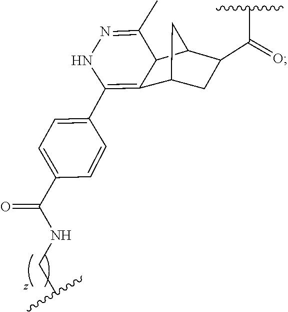
z is 1 to 6, preferably 1 to 5, more preferably 5; and/or
R³ is CH₂—F¹⁸, 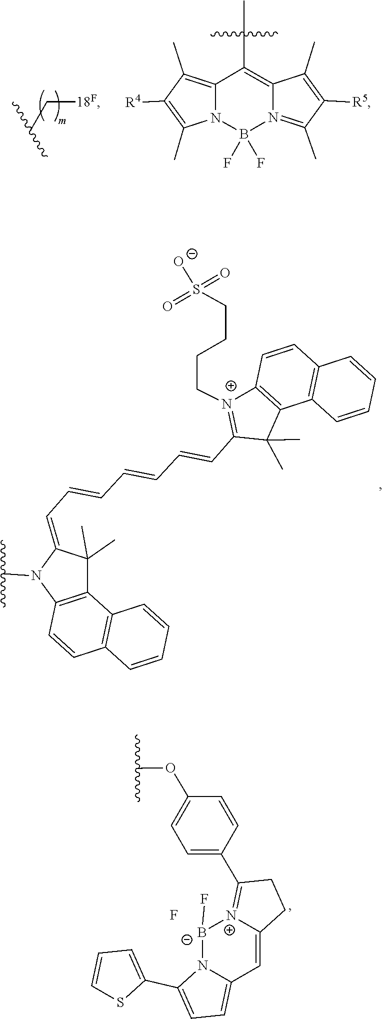
m is 2 to 4, preferably 2.

4. The compound of claim 1, wherein the compound is selected from the group consisting of

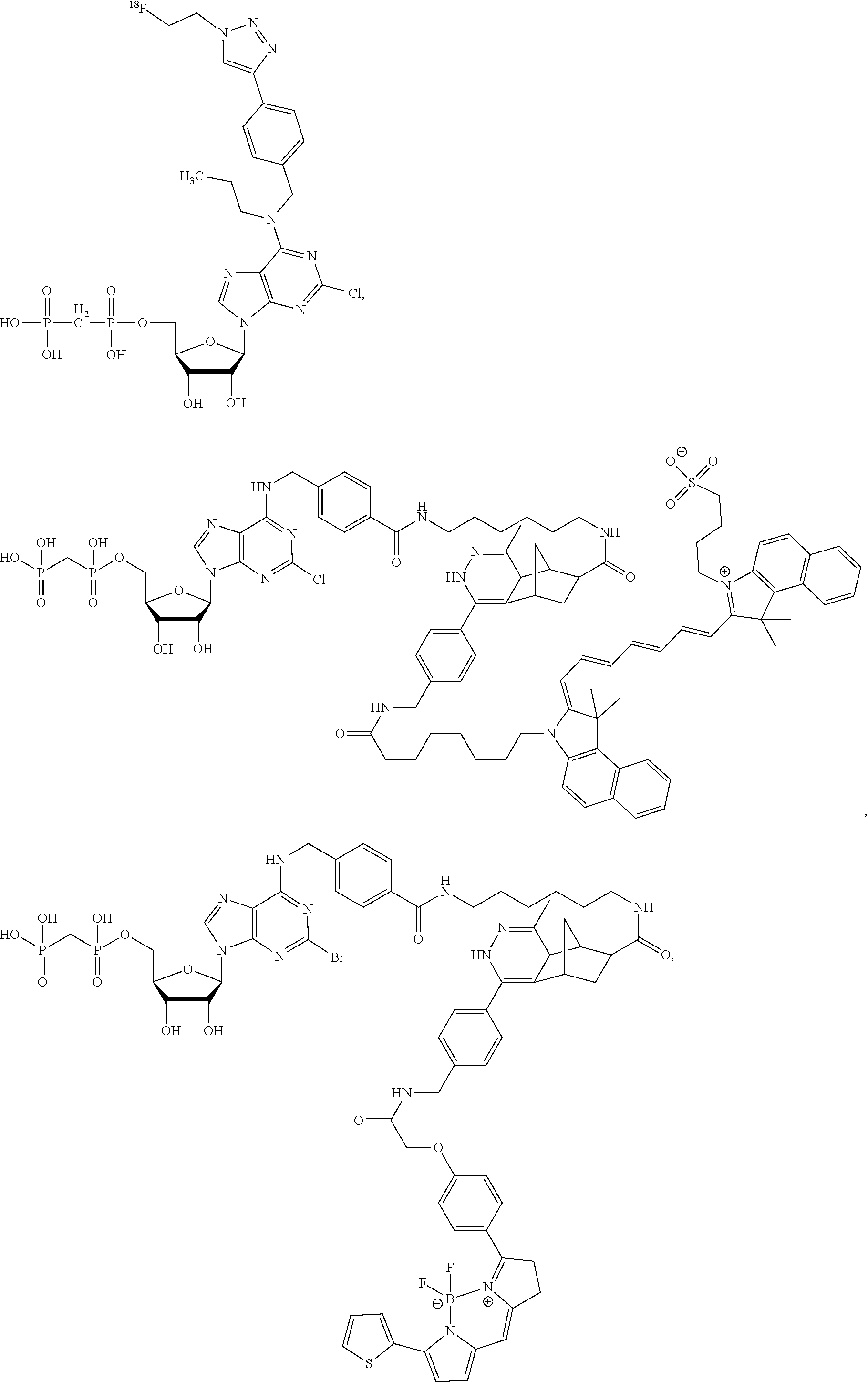

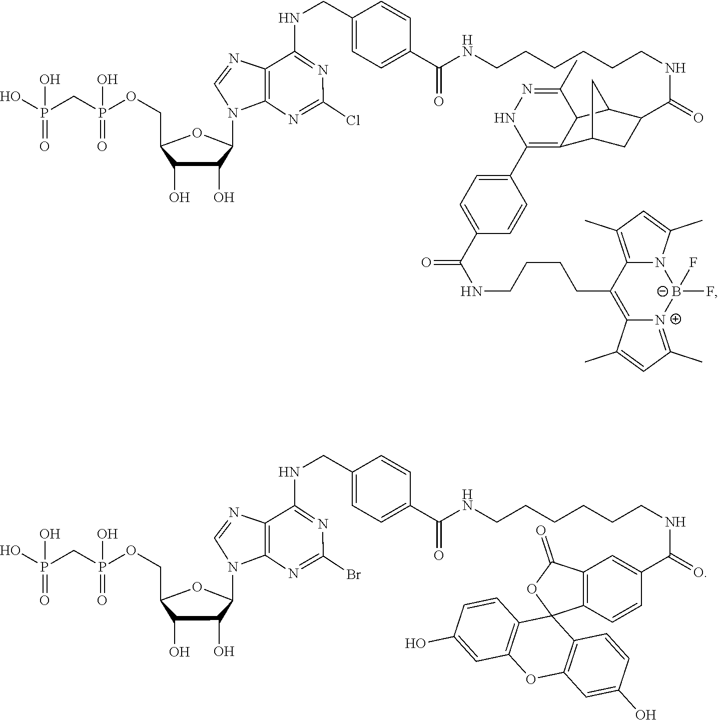

5. The compound of claim 1, wherein the compound is selected from the group consisting of

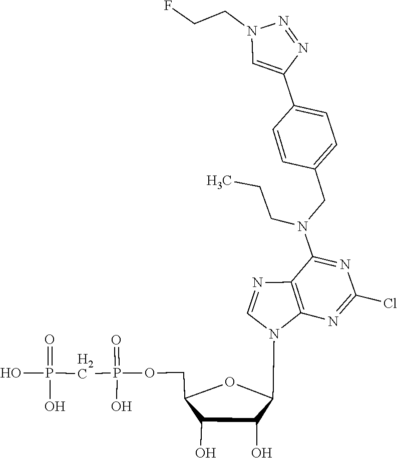

6. The compound of claim 1, wherein R³ is not Fluorescein

7. A pharmaceutical composition, comprising the compound of claim 1 and at least one pharmaceutically acceptable carrier.

8. A method for of treating a subject having a disease associated with increased CD73-expression comprising administering the compound of claim 1 to the subject.

9. (canceled)

10. The method of claim 8, wherein the disease is cancer, wherein the cancer is optionally a solid tumor, or an inflammatory disease.

11. The method of claim 10, wherein the cancer is selected from the group consisting of breast cancer, pancreatic cancer, colon cancer, lung cancer, kidney cancer, bladder cancer, prostate cancer, ovarian cancer, melanoma, glioma, head neck cancer and thyroid cancer.

12. The method of claim 10, wherein the inflammatory disease is multiple sclerosis or, rheumatoid arthritis.

13. A method of treating a subject having a disease associated with increased CD73-expression comprising administering a therapeutically effective dose of the pharmaceutical composition of claim 7 to the subject.

14. The method of claim 13, wherein the disease is cancer, wherein the cancer is optionally a solid tumor, or an inflammatory disease.

15. The method of claim 14, wherein the cancer is selected from the group consisting of breast cancer, pancreatic cancer, colon cancer, lung cancer, kidney cancer, bladder cancer, prostate cancer, ovarian cancer, melanoma, glioma, head neck cancer and thyroid cancer.

16. The method of claim 14, wherein the inflammatory disease is multiple sclerosis or rheumatoid arthritis.

17. The method of claim 13, wherein the compound of claim 1 in the composition is a compound of claim 5.

18. A method for of treating a subject having a disease associated with increased CD73-expression comprising administering the compound of claim 5 to the subject.

19. The method of claim 18, wherein the disease is cancer, wherein the cancer is optionally a solid tumor, or an inflammatory disease.

20. The method of claim 19, wherein the cancer is selected from the group consisting of breast cancer, pancreatic cancer, colon cancer, lung cancer, kidney cancer, bladder cancer, prostate cancer, ovarian cancer, melanoma, glioma, head neck cancer and thyroid cancer.

21. The method of claim 19, wherein the inflammatory disease is multiple sclerosis or rheumatoid arthritis.

22. A method for diagnosis of a subject having a disease associated with increased CD73-expression comprising administering the compound of claim 1 to the subject and detecting the compound in the subject.

23. The method of claim 22, wherein the disease is cancer, wherein the cancer is optionally a solid tumor, or an inflammatory disease.

24. The method of claim 23, wherein the cancer is selected from the group consisting of breast cancer, pancreatic cancer, colon cancer, lung cancer, kidney cancer, bladder cancer, prostate cancer, ovarian cancer, melanoma, glioma, head neck cancer and thyroid cancer.

25. The method of claim 24, wherein the inflammatory disease is multiple sclerosis or rheumatoid arthritis.
